(19) United States
(12) Patent Application Publication
(10) Pub. No.: US 2005/0222171 A1
(43) Pub. Date: Oct. 6, 2005

(54) Organic compounds

(76) Inventors: Guido Bold, Gipf-Oberfrick (CH); Andreas Floersheimer, Dornach (CH); Pascal Furet, Thann (FR); Patricia Imbach, Kaiseraugst (CH); Keiichi Masuya, Tsukuba-shi (JP); Joseph G. Schoepfer, Riehen (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/039,445

(22) Filed: Jan. 20, 2005

Related U.S. Application Data
(60) Provisional application No. 60/538,194, filed on Jan. 22, 2004.

(51) Int. Cl. A61K 31/519; C07D 487/04

(52) U.S. Cl. 514/259.3; 544/281

(57) ABSTRACT

The invention relates to the use of pyrazolo[1,5a]pyrimidin-7-yl amine compounds and salts thereof in the treatment of kinase dependent diseases and for the manufacture of pharmaceutical preparations for the treatment of said diseases, novel pyrazolo[1,5a]pyrimidin-7-yl amine compounds, and a process for the preparation of the novel pyrazolo[1,5a]pyrimidin-7-yl amine compounds.

### SUMMARY OF THE INVENTION

The invention relates to the use of pyrazolo[1,5a]pyrimidin-7-yl amine derivatives in the treatment of protein kinase dependent diseases, or for the manufacture of pharmaceutical compositions for use in the treatment of said diseases, methods of use of pyrazolo[1,5a]pyrimidin-7-yl amine derivatives in the treatment of said diseases, pharmaceutical preparations comprising pyrazolo[1,5a]pyrimidin-7-yl amine derivatives for the treatment of said diseases, novel pyrazolo[1,5a]pyrimidin-7-yl amine derivatives, processes for the manufacture of the novel pyrazolo[1,5a]pyrimidin-7-yl amine derivatives and pharmaceutical preparations, the use or methods of use of the pyrazolo[1,5a]pyrimidin-7-yl amine derivatives as mentioned above, and/or these pyrazolo[1,5a]pyrimidin-7-yl amine derivatives for use in the treatment of the animal or human body.

### BACKGROUND OF THE INVENTION

Pyrazolo[1,5-a]pyrimidin-7-yl-amine derivatives have been reported in the literature as ligands of benzodiazepine receptors (e.g., S. Selleri et al., Bioorg. Med. Chem 7 (12), 2705-11 (1999)), antagonists of the corticotropin releasing factor (EP 1097709), angiotensin II receptor antagonists (e.g., S. Takeshi et al., Japn. Pharm. Bull. 47 (7), 928-38 (1999)), monoxide synthetase inhibitors (JP 10101671), analgesics (WO 9535298), fungicides (EP 071792) or anti-inflammatory reagents (WO 9218504).

We have now found that the pyrazolo[1,5-a]pyrimidin-7-ylaminene residue can be also be used as a template for the design of potent kinase inhibitors.

In view of the large number of protein kinase inhibitors and the multitude of proliferative and other protein kinase-related diseases, there is an ever-existing need to provide novel classes of compounds that are useful as protein kinase inhibitors and thus in the treatment of related diseases.

What is desirable from the point of view of possible treatments of proliferative diseases is to have a plethora of compound classes each tailored to specific protein kinases or protein kinase classes, thus allowing to come to specific treatments. Therefore, a strong need exists to find new classes of compounds allowing for such specific inhibitory effects.

### SUMMARY OF THE INVENTION

The class of pyrazolo[1,5a]pyrimidin-7-yl amine compounds described herein, especially novel compounds falling under this class, has surprisingly been found to have pharmaceutically advantageous properties, allowing for the inhibition of specific types or classes or groups of kinases, especially c-Abl, Bcr-Abl, c-Kit, c-Raf, Flt-1, Flt-3, KDR, Her-1, PDGFR-kinase, c-Src, RET-receptor kinase, FGF-R1, FGF-R2, FGF-R3, FGF-R4, Ephrin receptor kinases (e.g., EphB2 kinase, EphB4 kinase and related Eph kinases), casein kinases (CK-1, CK-2, G-CK), Pak, ALK, ZAP70, Jak1, Jak2, Axl, Cdk1, cdk4, cdk5, Met, FAK, Pyk2, Syk, Insulin receptor kinase, Tie-2 or constitutively activating mutations of kinases (activating kinases) such as of Bcr-Abl, c-Kit, c-Raf, Flt-3, FGF-R3, PDGF-receptors, RET, and Met. The class of pyrazolo[1,5a]pyrimidin-7-yl amine compounds described herein further inhibit mutants of said kinases. In addition to this established activity, the pyrazolo[1,5a]pyrimidin-7-yl amine derivatives have the advantage in that their backbone allows for a plethora of substitution patterns that offer a broad possibility to achieve a fine tuning for specific interaction with the binding site of the targeted kinase or kinases, thus opening a new perspective and providing kinase inhibitors of various degrees of specificity. In view of these activities, the compounds can be used for the treatment of diseases related to especially aberrant or excessive activity of such types of kinases, especially those mentioned.

### DETAILED DESCRIPTION OF THE INVENTION

In one embodiment, the invention relates to the use of a compound of the formula (I): 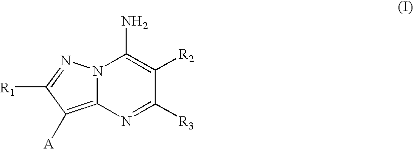
wherein:
R₂ is H; substituted or unsubstituted aryl; substituted or unsubstituted heteroaryl; substituted or unsubstituted aliphatic residue; a functional group; or a substituted or unsubstituted aryl, substituted or unsubstituted heteroaryl or substituted or unsubstituted aliphatic residue which is connected by one connecting group or atom to the pyrazolo[1,5a]pyrimidinyl ring;
R₃ can be H, substituted or unsubstituted aryl, substituted or unsubstituted heteroaryl, substituted or unsubstituted aliphatic residue, a functional group, or a substituted or unsubstituted aliphatic residue which may be connected by a connecting group or atom to the pyrazolo[1,5a]pyrimidinyl ring,
at least one of R₂ or R₃ is substituted or unsubstituted aryl; substituted or unsubstituted heteroaryl; or a substituted or unsubstituted heteroaryl or substituted or unsubstituted aryl residue which is connected by one connecting group or atom to the pyrazolo[1,5a]pyrimidinyl ring;
A is H, halogen (such as bromo), an aliphatic moiety, a functional group, substituted or unsubstituted aryl or substituted or unsubstituted heteroaryl; and
R₁ is H, halogen or lower alkyl,
or pharmaceutically acceptable salts thereof for treating a protein kinase dependent disease.

A preferred embodiment is the use of a compound according to the above,
wherein:
R₂ is H; lower alkyl; cycloalkyl; benzyl; benzo thienyl, indyl substituted by lower alkyl, pyridyl or thiazolyl optionally substituted by lower alkyl; unsubstituted phenyl or phenyl substituted by one or two substituents chosen from the group consisting of; halo, hydroxy, alkoxy, benzyloxy, cycloalkyl, amino, acetyl amino, lower alkyl sulfonamide and benzene sulfonamide substituted by one or two halo;
R3 is H; lower alkyl optionally substituted by halo; phenyl, pyridyl, or oxazolyl;
A is:
(a) H; halo; benzothienyl; pyridyl; methyl piperazinyl phenoxyl; indolyl substituted with lower alkyl;
(b) phenyl which is unsubstituted or substituted with one or more of the substituents chosen from the group consisting of; mono-, di- or tri-lower alkoxy, di-lower alkylaminyl, morpholinyl which is optionally di-substituted by alkyl,
piperazinyl which is substituted with one or more of the substituents chosen from the group consisting of lower alkyl, lower alkoxy, lower alkyl piperazinyl, pyrrolidinyl, dialkyl aminyl and lower alkanol; and
R₁ is H,
or pharmaceutically acceptable salts thereof for treating a protein kinase dependent disease.

A protein kinase dependent disease is preferably one that depends on c-Abl, Bcr-Abl, c-Kit, c-Raf, Flt-1, Flt-3, Her-1, KDR, PDGFR-kinase, c-Src, RET-receptor kinase, FGF-R1, FGF-R2, FGF-R3, FGF-R4, Ephrin receptor kinases (e.g., EphB2 kinase, EphB4 kinase and related Eph kinases), casein kinases (CK-1, CK-2, G-CK), Pak, ALK, ZAP70, Jak1, Jak2, Axl, Cdk1, cdk4, cdk5, Met, FAK, Pyk2, Syk, Insulin receptor kinase, Tie-2 or costitutively activating mutations of kinases (activating kinases) such as of Bcr-Abl, c-Kit, c-Raf, Flt-3, FGF-R3, PDGF-receptors, RET, and Met and (especially aberrantly highly expressed or activated) kinase-dependent disease or disease dependent on the activation of the kinase pathways, or a disease dependent on any two or more of the kinases just mentioned.

A protein kinase dependent disease is more preferably one that depends on c-abl, Flt-3, KDR, c-Src, RET, EphB4, c-kit, cdk1, FGFR-1, c-raf, Her-1, Ins-R or Tek.

Most preferably, the disease to be treated is a proliferative disease, preferably a benign or especially malignant tumor, more preferably carcinoma of the brain, kidney, liver, adrenal gland, bladder, breast, stomach (especially gastric tumors), ovaries, colon, rectum, prostate, pancreas, lung, vagina, thyroid, sarcoma, glioblastomas, multiple myeloma or gastrointestinal cancer, especially colon carcinoma or colorectal adenoma, or a tumor of the neck and head, an epidermal hyperproliferation, especially psoriasis, prostate hyperplasia, a neoplasia, especially of epithelial character, preferably mammary carcinoma, or a leukemia.

In a further embodiment, the disease to be treated is a disease which is triggered by persistent angiogenesis, such as psoriasis; Kaposi's sarcoma; restenosis, e.g., stent-induced restenosis; endometriosis; Crohn's disease; Hodgkin's disease; leukemia; arthritis, such as rheumatoid arthritis; hemangioma; angiofibroma; eye diseases, such as diabetic retinbpathy and neovascular glaucoma; renal diseases, such as glomerulonephritis; diabetic nephropathy; malignant nephrosclerosis; thrombotic microangiopathic syndromes; transplant rejections and glomerulopathy; fibrotic diseases, such as cirrhosis of the liver; mesangial cell-proliferative diseases; arteriosclerosis; injuries of the nerve tissue.

The compounds of the present invention can also be used for inhibiting the re-occlusion of vessels after balloon catheter treatment, for use in vascular prosthetics or after inserting mechanical devices for holding vessels open, such as, e.g., stents, as immunosuppressants, as an aid in scar-free wound healing, and for treating age spots and contact dermatitis.

In a further embodiment, the invention relates to a compound of formula (I): 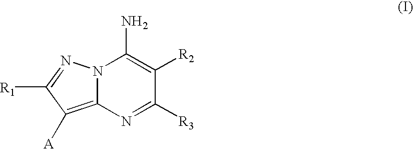
wherein:
R₂ is H; substituted or unsubstituted aryl; substituted or unsubstituted heteroaryl; an aliphatic residue; a functional group; or a substituted or unsubstituted aryl, substituted or unsubstituted heteroaryl or aliphatic residue which is connected by one connecting group or atom to the pyrazolo[1,5a]pyrimidinyl ring;
R₃ can be H, substituted or unsubstituted aryl, heteroaryl, an aliphatic residue, a functional group, or an aliphatic residue which may be connected by a connecting group or atom to the pyrazolo[1,5a]pyrimidinyl ring,
at least one of R₂ or R₃ is substituted or unsubstituted aryl; substituted or unsubstituted heteroaryl; or a substituted or unsubstituted heteroaryl or substituted or unsubstituted aryl residue which is connected by one connecting group or atom to the pyrazolo[1,5a]pyrimidinyl ring, and provided that both R₂ and A cannot both be unsubstituted phenyl;
A is H, halogen (such as bromo), an aliphatic moiety, a functional group, substituted or unsubstituted aryl or substituted or unsubstituted heteroaryl; and
R₁ is H, halogen or lower alkyl,
or a pharmaceutically acceptable salt thereof.

A preferred embodiment is a compound according to the above, wherein;
R₂ is H; lower alkyl; cycloalkyl; benzyl; benzo thienyl, indyl substituted by lower alkyl, pyridyl or thiazolyl optionally substituted by lower alkyl; unsubstituted phenyl or phenyl substituted by one or two substituents chosen from the group consisting of; halo, hydroxy, alkoxy, benzyloxy, cycloalkyl, amino, acetyl amino, lower alkyl sulfonamide and benzene sulfonamide substituted by one or two halo;
R3 is H; lower alkyl optionally substituted by halo; phenyl, pyridyl, or oxazolyl;
A is
(a) H; halo; benzothienyl; pyridyl; methyl piperazinyl phenoxyl; indolyl substituted with lower alkyl;
(b) phenyl which is unsubstituted or substituted with one or more of the substituents chosen from the group consisting of; mono-, di- or tri-lower alkoxy, di-lower alkylaminyl, morpholinyl which is optionally di-substituted by alkyl,
piperazinyl which is substituted with one or more of the substituents chosen from the group consisting of lower alkyl, lower alkoxy, lower alkyl piperazinyl, pyrrolidinyl, dialkyl aminyl and lower alkanol; and
R₁ is H; and provided that both R₂ and A cannot both be unsubstituted phenyl.

Most preferably, the compound is selected from the group consisting of:
3-{7-Amino-3-[4-(4-methyl-piperazin-1-yl)-phenyl]-pyrazolo[1,5-a]pyrimidin-6-yl}-phenol;
6-(3-benzyloxy-phenyl)-3-[4-(4-methyl-piperazin-1-yl)-phenyl]-pyrazolo[1,5-a]pyrimidin-7-yl}-phenol;
6-(3-Methoxy-phenyl)-3-[4-(4-methyl-piperazin-1-yl)-phenyl]-pyrazolo[1,5-a]pyrimidin-7-ylamine;
6-(3,5-Dimethoxy-phenyl)-3-[4-(4-methyl-piperazin-1-yl)-phenyl]-pyrazolo[1,5-a]pyrimidin-7-ylamine;
6-(3-Benzyloxy-phenyl)-3-[4-(4-methyl-piperazin-1-yl)-phenyl]-pyrazolo[1,5-a]pyrimidin-7-ylamine;
6-(4-Chloro-phenyl)-3-[4-(4-methyl-piperazin-1-yl)-phenyl]-pyrazolo[1,5-a]pyrimidin-7-ylamine;
6-(3-Chloro-phenyl)-3-[4-(4-methyl-piperazin-1-yl)-phenyl]-pyrazolo[1,5-a]pyrimidin-7-ylamine;
3-[4-(4-Methyl-piperazin-1-yl)-phenyl]-6-phenyl-pyrazolo[1,5-a]pyrimidin-7-ylamine;
5-Methyl-3-[4-(4-methyl-piperazin-1-yl)-phenyl]-6-phenyl-pyrazolo[1,5-a]pyrimidin-7-ylamine;
6-Methyl-3-[4-(4-methyl-piperazin-1-yl)-phenyl]-5-phenyl-pyrazolo[1,5-a]pyrimidin-7-ylamine;
N-{4-[7-Amino-3-(4-dimethylamino-phenyl)-pyrazolo[1,5-a]pyrimidin-6-yl]-phenyl}-2,3-dichloro-benzenesulfonamide;
4-Chloro-benzenesulfonic acid 4-[7-amino-3-(4-dimethylamino-phenyl)-pyrazolo[1,5-a]pyrimidin-6-yl]-phenyl ester;
6-(4-Methoxy-phenyl)-5-methyl-3-phenyl-pyrazolo[1,5-a]pyrimidin-7-ylamine;
3-(4-Methoxy-phenyl)-5-methyl-6-phenyl-pyrazolo[1,5-a]pyrimidin-7-ylamine;
6-(4-Bromo-phenyl)-3-(4-methoxy-phenyl)-5-methyl-pyrazolo[1,5-a]pyrimidin-7-ylamine;
6-(4-Bromo-phenyl)-5-methyl-3-phenyl-pyrazolo[1,5-a]pyrimidin-7-ylamine;
6-(2,6-Dichloro-phenyl)-3-phenyl-pyrazolo[1,5-a]pyrimidin-7-ylamine;
3-(3-Methoxy-phenyl)-6-phenyl-pyrazolo[1,5-a]pyrimidin-7-ylamine;
3-Bromo-5-phenyl-pyrazolo[1,5-a]pyrimidin-7-ylamine;
6-Benzo[b]thiophen-3-yl-3-[4-(4-methyl-piperazin-1-yl)-phenyl]-pyrazolo[1,5-a]pyrimidin-7-ylamine;
3-(4-Bromo-phenyl)-5-phenyl-pyrazolo[1,5-a]pyrimidin-7-ylamine;
3-[4-(4-Methyl-piperazin-1-yl)-phenyl]-6-thiophen-3-yl-pyrazolo[1,5-a]pyrimidin-7-ylamine;
3-Benzo[b]thiophen-3-yl-6-(3-methoxy-phenyl)-pyrazolo[1,5-a]pyrimidin-7-ylamine;
6-Benzo-3-[4-(4-methyl-piperazin-1-yl)-phenyl]-pyrazolo[1,5-a]pyrimidin-7-ylamine;
6-(3-Methoxy-phenyl)-3-[3-(4-methyl-piperazin-1-yl)-phenyl]-pyrazolo[1,5-a]pyrimidin-7-ylamine;
6-(1-Methyl-1H-indol-3-yl)-3-[4-(4-methyl-piperazin-1-yl)-phenyl]-pyrazolo[1,5-a]pyrimidin-7-ylamine;
6-(4-Methoxy-phenyl)-3-[4-(4-methyl-piperazin-1-yl)-phenyl]-pyrazolo[1,5-a]pyrimidin-7-ylamine;
6-(2-Methoxy-phenyl)-3-[4-(4-methyl-piperazin-1-yl)-phenyl]-pyrazolo[1,5-a]pyrimidin-7-ylamine;
6-(3-Methoxy-phenyl)-3-pyridin-3-yl-pyrazolo[1,5-a]pyrimidin-7-ylamine;
3-{7-Amino-3-[3-(4-methyl-piperazin-1-yl)-phenyl]-pyrazolo[1,5-a]pyrimidin-6-yl}-phenol;
6-(3-Benzyloxy-phenyl)-3-[2-methoxy-5-(4-methyl-piperazin-1-yl)-phenyl]-pyrazolo[1,5-a]pyrimidin-7-ylamine;
3-{7-Amino-3-[2-methoxy-5-(4-methyl-piperazin-1-yl)-phenyl]-pyrazolo[1,5-a]pyrimidin-6-yl}-phenol;
6-(2-Benzyloxy-phenyl)-3-[4-(4-methyl-piperazin-1-yl)-phenyl]-pyrazolo[1,5-a]pyrimidin-7-ylamine;
2-{7-Amino-3-[4-(4-methyl-piperazin-1-yl)-phenyl]-pyrazolo[1,5-a]pyrimidin-6-yl}-phenol;
6-(4-Benzyloxy-phenyl)-3-[4-(4-methyl-piperazin-1-yl)-phenyl]-pyrazolo[1,5-a]pyrimidin-7-ylamine;
4-{7-Amino-3-[4-(4-methyl-piperazin-1-yl)-phenyl]-pyrazolo[1,5-a]pyrimidin-6-yl}-phenol;
6-(2-Benzyloxy-phenyl)-3-[3-(4-methyl-piperazin-1-yl)-phenyl]-pyrazolo[1,5-a]pyrimidin-7-ylamine;
2-{7-Amino-3-[3-(4-methyl-piperazin-1-yl)-phenyl]-pyrazolo[1,5-a]pyrimidin-6-yl}-phenol;
6-(4-Benzyloxy-phenyl)-3-[3-(4-methyl-piperazin-1-yl)-phenyl]-pyrazolo[1,5-a]pyrimidin-7-ylamine;
4-{7-Amino-3-[3-(4-methyl-piperazin-1-yl)-phenyl]-pyrazolo[1,5-a]pyrimidin-6-yl}-phenol;
6-(2-Benzyloxy-phenyl)-3-[2-methoxy-5-(4-methyl-piperazin-1-yl)-phenyl]-pyrazolo[1,5-a]pyrimidin-7-ylamine;
2-{7-Amino-3-[2-methoxy-5-(4-methyl-piperazin-1-yl)-phenyl]-pyrazolo[1,5-a]pyrimidin-6-yl}-phenol;
6-(4-Benzyloxy-phenyl)-3-[2-methoxy-5-(4-methyl-piperazin-1-yl)-phenyl)-pyrazololo[1,5-a]pyrimidin-7-ylamine;
4-{7-Amino-3-[2-methoxy-5-(4-methyl-piperazin-1-yl)-phenyl]-pyrazolo[1,5-a]pyrimidin-6-yl}-phenol;
6-(3-Benzyloxy-phenyl)-3-[1-methyl-1H-indol-3-yl)-pyrazolo[1,5-a]pyrimidin-7-ylamine;
3-[7-Amino-3-(1-methyl-1H-indol-3-yl)-pyrazolo[1,5-a]pyrimidin-6-yl]-phenol;
3-[7-Amino-3-pyridin-3-yl-pyrazolo[1,5-a]pyrimidin-6-yl]-phenol;
6-(3-Benzyloxy-phenyl)-3-(2-methoxy-phenyl)-pyrazolo[1,5-a]pyrimidin-7-ylamine;
3-[7-Amino-3-(2-methoxy-phenyl)-pyrazolo[1,5-a]pyrimidin-6-yl]-phenol;
3-[3-(4-Methyl-piperazin-1-yl)-phenyl]-6-thiophen-3-yl-pyrazolo[1,5-a]pyrimidin-7-ylamine;
3-(2-Methoxy-5-(4-methyl-piperazin-1-yl)-phenyl]-6-thiophen-3-yl-pyrazolo[1,5-a]pyrimidin-7-ylamine;
3-[4-(4-Methyl-piperazin-1-yl)-phenyl]-6-pyridin-4-yl-pyrazolo[1,5-a]pyrimidin-7-ylamine;
6-(3-Amino-phenyl)-3-[3-(4-methyl-piperazin-1-yl)-phenyl]-pyrazolo[1,5-a]pyrimidin-7-ylamine;
6-(3-Amino-phenyl)-3-[4-(4-methyl-piperazin-1-yl)-phenyl]-pyrazolo[1,5-a]pyrimidin-7-ylamine;
6-(2-Amino-phenyl)-3-[4-(4-methyl-piperazin-1-yl)-phenyl]-pyrazolo[1,5-a]pyrimidin-7-ylamine;
3-[4-(4-Methyl-piperazin-1-yl)-phenyl]-6-(4-methyl-thiazol-2-yl)-pyrazolo[1,5-a]pyrimidin-7-ylamine;
6-Benzo[b]thiophen-3-yl-3-[2-methoxy-5-(4-methyl-piperazin-1-yl)-phenyl]-pyrazolo[1,5-a]pyrimidin-7-ylamine;
6-Benzo[b]thiophen-3-yl-3-[4-methoxy-phenyl)-pyrazolo[1,5-a]pyrimidin-7-ylamine;
3-(3-Methoxy-phenyl)-6-thiophen-3-yl-pyrazolo[1,5-a]pyrimidin-7-ylamine;
6-(3-Benzyloxy-phenyl)-3-(3-methoxy-phenyl)-pyrazolo[1,5-a]pyrimidin-7-ylamine;
3-[7-Amino-3-(3-methoxy-phenyl)-pyrazolo[1,5-a]pyrimidin-6-yl]-phenol;
(4-{7-Amino-3-[4-(4-methyl-piperazin-1-yl)-phenyl]-pyrazolo[1,5-a]pyrimidin-6-yl}-phenyl)-carbamic acid ethyl ester
6-(3-Chloro-phenyl)-5-methyl-3-[3-(4-methyl-piperazin-1-yl)-phenyl]-pyrazolo[1,5-a]pyrimidin-7-ylamine;
6-(3-Chloro-phenyl)-5-methyl-3-[4-(4-methyl-piperazin-1-yl)-phenyl]-pyrazolo[1,5-a]pyrimidin-7-ylamine;
6-(3-Chloro-phenyl)-3-[2-methoxy-5-(4-methyl-piperazin-1-yl)-phenyl]-5-methyl-pyrazolo[1,5-a]pyrimidin-7-ylamine;
6-(3-Chloro-phenyl)-3-[2-methoxy-4-(4-methyl-piperazin-1-yl)-phenyl]-5-methyl-pyrazolo[1,5-a]pyrimidin-7-ylamine;
3-{7-Amino-3-[2-methoxy-4-(4-methyl-piperazin-1-yl)-phenyl]-pyrazolo[1,5-a]pyrimidin-7-phenol;
6-(2-Chloro-phenyl)-3-[4-(4-methyl-piperazin-1-yl)-phenyl]-pyrazolo[1,5-a]pyrimidin-7-ylamine;
6-(2-Chloro-phenyl)-3-[3-(4-methyl-piperazin-1-yl)-phenyl]-pyrazolo[1,5-a]pyrimidin-7-ylamine;
6-(4-Fluoro-phenyl)-5-methyl-3-[4-(4-methyl-piperazin-1-yl)-phenyl]-pyrazolo[1,5-a]pyrimidin-7-ylamine;
6-(4-Fluoro-phenyl)-5-methyl-3-[3-(4-methyl-piperazin-1-yl)-phenyl]-pyrazolo[1,5-a]pyrimidin-7-ylamine;
6-(3-Chloro-phenyl)-5-methyl-3-{3-[4-(1-methyl-piperidin-4-yl)-piperazin-1-yl]-phenyl]-pyrazolo[1,5-a]pyrimidin-7-ylamine;
6-(3-Chloro-4-fluoro-phenyl)-5-methyl-3-[3-(4-methyl-piperazin-1-yl)-phenyl]pyrazolo[1,5-a]pyrimidin-7-ylamine;
6-(3-Chloro-4-fluoro-phenyl)-5-methyl-3-[4-(4-methyl-piperazin-1-yl)-phenyl]-pyrazolo[1,5-a]pyrimidin-7-ylamine;
6-(3-Bromo-phenyl)-5-methyl-3-[3-(4-methyl-piperazin-1-yl)-phenyl]-pyrazolo[1,5-a]pyrimidin-7-ylamine;
6-(3-Bromo-benzyl)-3-[3-(4-methyl-piperazin-1-yl)-phenyl]-pyrazolo[1,5-a]pyrimidin-7-ylamine;
6-(3-Bromo-phenyl)-3-[3-(4-methyl-piperazin-1-yl)-phenyl]-pyrazolo[1,5-a]pyrimidin-7-ylamine;
6-(3-Chloro-phenyl)-5-methyl-3-(3-morpholin-4-yl-phenyl)-pyrazolo[1,5-a]pyrimidin-7-ylamine; 6-(3-Chloro-phenyl)-3-(4-methoxy-phenyl)-5-methyl-pyrazolo[1,5-a]pyrimidin-7-ylamine;
6-(3-Chloro-phenyl)-3-[3-((2R,6S)-2,6-dimethyl-morpholin-4-yl)-phenyl]-5-methyl-pyrazolo[1,5-a]pyrimidin-7-ylamine;
2-(4-{3-[7-Amino-6-(3-chloro-phenyl)-5-methyl-pyrazolo[1,5-a]pyrimidin-3-yl]-phenyl}-piperazin-1-yl)-ethanol;
6-Benzyl-3-[3-(4-methyl-piperazin-1-yl)-phenyl]-pyrazolo[1,5-a]pyrimidin-7-ylamine;
6-(3-Chloro-phenyl)-3-(3,4-dimethoxy-phenyl)-5-fluoromethyl-pyrazolo[1,5-a]pyrimidin-7-ylamine;
6-(3-Chloro-phenyl)-3-(3,4-dimethoxy-phenyl)-5-methyl-pyrazolo[1,5-a]pyrimidin-7-ylamine;
6-(3-Chloro-4-fluoro-phenyl)-3-(3,4-dimethoxy-phenyl)-5-methyl-pyrazolo[1,5-a]pyrimidin-7-ylamine;
6-(3-Chloro-4-fluoro-phenyl)-3-(4-methoxy-phenyl)-5-methyl-pyrazolo[1,5-a]pyrimidin-7-ylamine;
6-(4-Fluoro-phenyl)-3-(4-methoxy-phenyl)-5-methyl-pyrazolo[1,5-a]pyrimidin-7-ylamine;
2-(4-{3-[7-Amino-6-(4-fluoro-phenyl)-5-methyl-pyrazolo[1,5-a]pyrimidin-3-yl]-phenyl}-piperazin-1-yl)-ethanol;
6-(3,4-Difluoro-phenyl)-5-methyl-3-[3-(4-methyl-piperazin-1-yl)-phenyl]-pyrazolo[1,5-a]pyrimidin-7-ylamine;
6-(3,4-Difluoro-phenyl)-3-(3,4-dimethoxy-phenyl)-5-methyl-pyrazolo[1,5-a]pyrimidin-7-ylamine;
2-(4-{3-[7-Amino-6-(3-chloro-4-fluoro-phenyl)-5-methyl-pyrazolo[1,5-a]pyrimidin-3-yl]-phenyl}-piperazin-1-yl)-ethanol;
2-(4-{3-[7-Amino-6-(3,4-difluoro-phenyl)-5-methyl-pyrazolo[1,5-a]pyrimidin-3-yl]-phenyl}-piperazin-1-yl)-ethanol;
6-(3-Chloro-phenyl)-5-methyl-3-[3-(4-pyrrolidin-1-yl-piperidin-1-yl)-phenyl]-pyrazolo[1,5-a]pyrimidin-7-ylamine;
6-(4-Fluoro-phenyl)-5-methyl-3-[3-(4-pyrrolidin-1-yl-piperidin-1-yl)-phenyl]-pyrazolo[1,5-a]pyrimidin-7-ylamine;
6-(3-Chloro-phenyl)-3-[3-(4-diethylamino-piperidin-1-yl)-phenyl]-5-methyl-pyrazolo[1,5-a]pyrimidin-7-ylamine;
3-[3-(4-Diethylamino-piperidin-1-yl)-phenyl]-6-(4-fluoro-phenyl)-5-methyl-pyrazolo[1,5-a]pyrimidin-7-ylamine;
6-(4-Fluoro-phenyl)-5-methyl-3-[3-(4-methyl-4-oxy-piperazin-1-yl)-phenyl]-pyrazolo[1,5-a]pyrimidin-7-ylamine;
6-(4-Fluoro-phenyl)-5-methyl-3-[3-(4-methyl-1,4-dioxy-piperazin-1-yl)-phenyl]-pyrazolo[1,5-a]pyrimidin-7-ylamine;
6-(3-Chloro-phenyl)-3-[3-(4-dimethylamino-piperidin-1-yl)-phenyl]-5-methyl-pyrazolo[1,5-a]pyrimidin-7-ylamine;
6-(3,4-Difluoro-phenyl)-3-[3-(4-dimethylamino-piperidin-1-yl)-phenyl]-5-methyl-pyrazolo[1,5-a]pyrimidin-7-ylamine;
6-(3-Chloro-phenyl)-5-methyl-3-(3,4,5-trimethoxy-phenyl)-pyrazolo[1,5-a]pyrimidin-7-ylamine; 6-(3,4-Difluoro-phenyl)-5-methyl-3-(3,4,5-trimethoxy-phenyl)-pyrazolo[1,5-a]pyrimidin-7-ylamine;
6-(3-Chloro-phenyl)-3-(3-methoxy-phenyl)-5-methyl-pyrazolo[1,5-a]pyrimidin-7-ylamine;
6-[7-Amino-3-(3,4-dimethoxy-phenyl)-pyrazolo[1,5-a]pyrimidin-6-yl]-pyridin-2-ol;
6-Benzyl-3-(3,4-dimethoxy-phenyl)-pyrazolo[1,5-a]pyrimidin-7-ylamine;
3-(3,4-Dimethoxy-phenyl)-6-(3-fluoro-benzyl)-pyrazolo[1,5-a]pyrimidin-7-ylamine; and
pharmaceutically acceptable salts thereof.

Yet another embodiment is the use of a compound according to the above in the preparation of a pharmaceutical composition.

Yet another embodiment is a pharmaceutical composition comprising a compound according to the above.

The pharmaceutical composition preferably comprises a compound according to the above and an acceptable pharmaceutical carrier.

In another embodiment, there is provided the use of a compound according to the above in the preparation of a pharmaceutical compositions for use in the treatment of a kinase dependent disease.

A further embodiment is a process to prepare a compound according to the above comprising:
(a) reacting a nitrile, A-CH₂—C≡N, with ethyl formate in the presence of an organic solvent to form a substituted 3-oxo-propionitrile,
(b) condensing the substituted 3-oxo-propionitriles of step (a) with hydrazine monohydrate in an organic solvent to form a 2H-pyrazol-3-ylamine of formula (III): 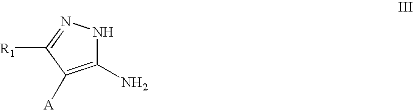
(d) formylating a substituted nitrile in the presence of ethanolate and formic acid ethyl ester to prepare a 3-oxo-propionitrile of formula (II): 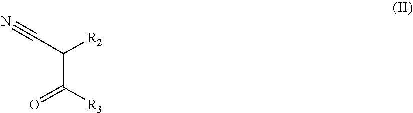
(c) condensing the 3-oxo-propionitrile of formula (II) and the 2H-pyrazol-3-ylamines of formula (III) in the presence of an organic solvent to form a compound of formula (I).

The invention in particular relates to pyrazolo[1,5a]pyrimidin-7-yl amine compounds of the formula (I): 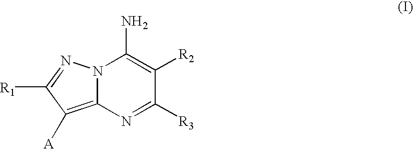
wherein:
R₂ is H; substituted or unsubstituted aryl; substituted or unsubstituted heteroaryl; substituted or unsubstituted aliphatic residue; a functional group; or a substituted or unsubstituted aryl, substituted or unsubstituted heteroaryl or substituted or unsubstituted aliphatic residue which is connected by one connecting group or atom to the pyrazolo[1,5a]pyrimidinyl ring;
R₃ is H, substituted or unsubstituted aryl, substituted or unsubstituted heteroaryl, substituted or unsubstituted aliphatic residue, a functional group, or an aliphatic residue which may be connected by a connecting group or atom to the pyrazolo[1,5a]pyrimidinyl ring, at least one of R₂ or R₃ is substituted or unsubstituted aryl; substituted or unsubstituted heteroaryl; or a substituted or unsubstituted heteroaryl or substituted or unsubstituted aryl residue which is connected by one connecting group or atom to the pyrazolo[1,5a]pyrimidinyl ring;
A is H, halogen (such as bromo), an aliphatic moiety, a functional group, substituted or unsubstituted aryl, or substituted or unsubstituted heteroaryl; and
R₁ is H, halogen or lower alkyl,
or pharmaceutically acceptable salts thereof,
in the treatment of protein kinase (especially tyrosine protein kinase) dependent diseases or for the manufacture of pharmaceutical compositions for use in the treatment of said diseases, methods of use of compounds of formula (I) in the treatment of said diseases, or pharmaceutical preparations comprising compounds of formula (I) for the treatment of said diseases.

The present invention is especially related to a compound of formula (I) wherein R₂ is H; substituted or unsubstituted aryl; substituted or unsubstituted heteroaryl; substituted or unsubstituted aliphatic residue; a functional group; or a substituted or unsubstituted aryl, substituted or unsubstituted heteroaryl or substituted or unsubstituted aliphatic residue which is connected by one connecting group or atom to the pyrazolo[1,5a]pyrimidinyl ring;
R₃ is H, substituted or unsubstituted aryl, substituted or unsubstituted heteroaryl, substituted or unsubstituted aliphatic residue, a functional group, or an aliphatic residue which may be connected by a connecting group or atom to the pyrazolo[1,5a]pyrimidinyl ring,
at least one of R₂ or R₃ is substituted or unsubstituted aryl; substituted or unsubstituted heteroaryl; or a substituted or unsubstituted heteroaryl or substituted or unsubstituted aryl residue which is connected by one connecting group or atom to the pyrazolo[1,5a]pyrimidinyl ring, and provided that R₂ and A cannot both be unsubstituted phenyl;
A is H, halogen (such as bromo), an aliphatic moiety, a functional group, substituted or unsubstituted aryl or heteroaryl; and
R₁ is H, halogen or lower alkyl,or pharmaceutically acceptable salts thereof,
in the treatment of protein kinase (especially tyrosine protein kinase) dependent diseases or for the manufacture of pharmaceutical compositions for use in the treatment of said diseases, methods of use of compounds of formula (I) in the treatment of said diseases, pharmaceutical preparations comprising compounds of formula (I) for the treatment of said diseases, compounds of formula (I) for use in the treatment of said diseases.

The present invention also relates to a method of treating kinase dependent diseases comprising administering pyrazolo[1,5a]pyrimidin-7-yl amine compounds of the formula (I) to a warm-blooded animal, especially a human. The present invention also relates to pharmaceutical preparations comprising an pyrazolo[1,5a]pyrimidin-7-yl amine compound of the formula (I), especially for the treatment of a kinase dependent disease, novel pyrazolo[1,5a]pyrimidin-7-yl amine compounds of the formula (I), a process for the manufacture of the pyrazolo[1,5a]pyrimidin-7-yl amine compounds of the formula (I), and novel starting materials and intermediates for their manufacture. The present invention also relates to use of a compound of formula 1 in the manufacture of a pharmaceutical preparation for the treatment of a kinase dependent disease.

The general terms used hereinbefore and hereinafter preferably have within the context of this disclosure the following meanings, unless otherwise indicated:

“Aryl” is an aromatic radical having 6 to 14 carbon atoms, especially phenyl, naphthyl, indenyl, azulenyl, or anthryl, and is unsubstituted or substituted by one or more, preferably one or two substituents, wherein the substituents are selected from any of the functional groups defined below, and including: lower halo, alkyl, substituted alkyl, halo lower alkyl e.g. trifluoromethyl, lower alkenyl, lower alkynyl, lower alkanoyl, lower alkoxy, hydroxy, another aryl, etherified or esterified hydroxy, amino, mono- or disubstituted amino, amino lower alkyl, amino lower alkoxy; acetyl amino; amidino, halogen, nitro, cyano, cyano lower alkyl, carboxy, esterified carboxy especially lower alkoxy carbonyl, e.g. methoxy carbonyl, n-propoxy carbonyl or iso-propoxy carbonyl, alkanoyl, benzoyl, carbamoyl, N-mono- or N,N-disubstituted carbamoyl, carbamates, alkyl carbamic acid esters, amidino, guanidino, urea, ureido, mercapto, sulfo, lower alkylthio, sulfoamino, sulfonamide, benzosulfonamide, sulfonate, phenyl, benzyl, phenoxy, benzyloxy, phenylthio, phenyl-lower alkylthio, alkylphenylthio, lower alkylsulfinyl, phenylsulfinyl, phenyl-lower alkylsulfinyl, alkylphenylsulfinyl, lower alkanesulfonyl, phenylsulfonyl, phenyl-lower alkylsulfonyl, alkylphenylsulfonyl, halogen-lower alkylmercapto, halogen-lower alkylsulfonyl, such as especially trifluoromethane sulfonyl, dihydroxybora (—B(OH)₂), heterocyclyl, and lower alkylene dioxy bound at adjacent C-atoms of the ring, such as methylene dioxy, phosphono (—P(═O)(OH)₂), hydroxy-lower alkoxy phosphoryl or di-lower alkoxyphosphoryl, carbamoyl, mono- or-di-lower alkylcarbamoyl, mono- or di-(hydroxy-lower alkyl)-carbamoyl, or —NR₄R₅, wherein R₄ and R₅ can be the same or different and are independently H; lower alkyl (e.g. methyl, ethyl or propyl); or R₄ and R₅ together with the N atom form a 3- to 8-membered heterocyclic ring containing 1-4 nitrogen, oxygen or sulfur atoms (e.g. piperazinyl, lower alkyl-piperazinyl, azetidinyl, pyrrolidinyl, piperidino, morpholinyl, imidazolinyl).

Aryl is more preferably phenyl which is either unsubstituted or independently substituted by one or two substituents selected from a solubilizing group selected from the group consisting of: halo (such as Cl, Br or F); hydroxy; lower alkyl (such as C₁-C₃ lower alkyl or methyl); aryl (such as phenyl or benzyl); amino; amino lower alkyl (such as dimethylamino); acetyl amino; amino lower alkoxy (such as ethoxyamine); substituted lower alkyl (such as fluror ethyl); alkoxy (such as methoxy or benzyloxy where the benzyl ring may be substituted or unsubstituted, such as 3,4-dichlorobenzyloxy); sulfoamino; substituted or unsubstituted sulfonamide (such as benzo sulfonamide, chlorobenzene sulfonamide or 2,3-dichloro benzene sulfonamide); substituted or unsubstituted sulfonate (such as chloro-phenyl sulfonate); substituted urea (such as 3-trifluoro-methyl-phenyl urea or 4-morpholin-4-yl-3-triflurormethyl-phenyl-urea); alkyl carbamic acid ester or carbamates (such as ethyl-N-phenyl-carbamate) or —NR₄R₅, wherein R₄ and R₅ can be the same or different and are independently H; lower alkyl (e.g. methyl, ethyl or propyl); or R₄ and R₅ together with the N atom form a 3- to 8-membered heterocyclic ring containing 1-4 nitrogen, oxygen or sulfur atoms (e.g. piperazinyl, lower alkyl-piperazinyl, pyridyl, indolyl, thiophenyl, thiazolyl, morpholinyl n-methyl piperazinyl, benzothiophenyl, azetidinyl, pyrrolidinyl, piperidino or imidazolinyl) where when R₄ and R₅ together with the N form an heterocyclic ring, said ring may be substituted with 1, 2 or more of any of the substituents described herein, preferably piperazinyl, pyrrolidinyl, alkyl such as methyl, or hydroxy alkyl such as ethanyl. Examples of the heteroring formed by R₄ and R₅ together with the N include morpholinyl, which can be unsubstituted or substituted with methyl or dimethyl; piperazinyl which can be unsubstituted or substituted with 1, 2 or 3 substituents prefereably methyl, oxy or ethanol; or piperadinyl which can be unsubstituted or substituted with 1, 2 or 3 substituents prefereably pyrrolidinyl, amine, alkyl amine, methyl amine, dialkyl amine, dimethylamine or diethylamine;

A heteroaryl group is preferably monocyclic, but may be bi- or tri-cyclic, and comprises 3-24, preferably 4-16 ring atoms, wherein at least one or more, preferably one to four ring carbons are replaced by a heteroatom selected from O, N or S. Preferably the heteroaryl group is selected from pyridyl, indolyl, pyrimidyl, pyrazolyl, oxazolyl, thiophenyl, benzothiophenyl, 2H-pyrrolyl, pyrrolyl, imidazolyl, benzimidazolyl, pyrazolyl, indazolyl, purinyl, pyrazinyl, pyridazinyl, 4H-quinolizinyl, isoquinolyl, quinolyl, phthalazinyl, naphthyridinyl, quinoxalyl, quinazolinyl, quinnolinyl, indolizinyl, 3H-indolyl, isoindolyl, isoxazolyl, thiazolyl, isothiazolyl, triazolyl, tetrazolyl, furazanyl and benzo[d]pyrazol.

More preferably the heteroaryl group is selected from the group consisting of pyridyl, indolyl, pyrimidyl, pyrazolyl, oxazolyl, thiophenyl or benzothiophenyl.

The heteroaryl group may be unsubstituted or substituted by one or more substituents selected from the group defined above as substituents for aryl, most preferably by hydroxy, halogen, lower alkyl, such as methyl or lower alkoxy, such as methoxy or ethoxy.

Aliphatic as used herein refers to any non-aromatic carbon based residue. Examples of aliphatic residues include substituted or unsubstituted alkyl, cycloalkyl, alkenyl and alkynyl.

Alkyl includes lower alkyl preferably alkyl with up to 7 carbon atoms, preferably from 1 to and including 5, and is linear or branched; preferably, lower alkyl is pentyl, such as n-pentyl, butyl, such as n-butyl, sec-butyl, isobutyl, tert-butyl, propyl, such as n-propyl or isopropyl, ethyl or methyl. Preferably lower alkyl is methyl, propyl or tert-butyl.

A cycloalkyl group is preferably cyclopentyl, cyclohexyl or cycloheptyl, and may be unsubstituted or substituted by one or more, especially one or two, substituents selected from the group defined above as substituents for aryl, most preferably by lower alkyl such as methyl, lower alkoxy such as methoxy or ethoxy, or hydroxy.

Alkenyl and alkynyl preferably have up to 7 carbon atoms, preferably from 1 to and including 5, and can be linear or branched.

Alkyl, cycloalkyl, alkenyl and alkynyl can be substituted or unsubstituted, and when substituted may be with up to 3 substituents including other alkyl, cycloalkyl, alkenyl, alkynyl, any of the substituents defined above for aryl or any of the functional groups defined below.

Halo or halogen is preferably fluoro, chloro, bromo or iodo, most preferably fluoro, chloro or bromo.

The term “connecting atom or group” as used herein includes alkyl, (such as —CH₂—); oxy —O—; keto —CO—; thio —S—; sulfonyl —SO₂—; sulfoxides —SO—; amines —NH— or —NR—; carboxylic acid; alcohol; esters (—COO—); amides (—CONR—, —CONHR′—); sulfonamides (—SO₂NH—, —SO₂NR′—); sulfones (—SO₂—); sulfoxides (—SO—); amino-group; ureas (—NH—CO—NH—, —NR—CO—NH—, —NH—CO—NR—, —NR—CO—NR—); ethers (—O—); carbamates (—NH—CO—O—, —NR—CO—O—); or inverse amides sulfonamides and esters (—NH—CO—, —NR—CO—, —NH—SO₂—, —NR—SO₂—, —OOC—).

The term “functional group” as used herein includes: carboxylic acid; hydroxyl; halogen; cyano (—CN); ethers (—OR); ketones (—CO—R); esters (—COOR); amides (—CONH2, —CONHR, —CONRR′); thioethers (—SR); sulfonamides (—SO₂NH₂, —SO₂NHR, —SO₂NRR′); sulfones (—SO₂—R); sulfoxides (—SO—R); amines (—NHR, NR′R); ureas (—NH—CO—NH₂, —NH—CO—NHR); ethers (—O—R); halogens; carbamates (—NH—CO—OR); aldehyde-function (—CHO); then also inverse amides; sulfonamides and esters (—NH—CO—R, —NH—SO₂—R, —OOC—R);
R and R′ are the same are different and may be H or are any aliphatic, aryl or heteroaryl moiety as defined above.

Where the plural form is used for compounds, salts, pharmaceutical preparations, diseases and the like, this is intended to mean also a single compound, salt, or the like.

Salts are especially the pharmaceutically acceptable salts of compounds of formula I.

Such salts are formed, for example, as acid addition salts, preferably with organic or inorganic acids, from compounds of formula (I) with a basic nitrogen atom, especially the pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, for example acetic acid, propionic acid, octanoic acid, decanoic acid, dodecanoic acid, glycolic acid, lactic acid, fumaric acid, succinic acid, adipic acid, pimelic acid, suberic acid, azelaic acid, malic acid, tartaric acid, citric acid, amino acids, such as glutamic acid or aspartic acid, maleic acid, hydroxymaleic acid, methylmaleic acid, cyclohexanecarboxylic acid, adamantanecarboxylic acid, benzoic acid, salicylic acid, 4-aminosalicylic acid, phthalic acid, phenylacetic acid, mandelic acid, cinnamic acid, methane- or ethane-sulfonic acid, 2-hydroxyethanesulfonic acid, ethane-1,2-disulfonic acid, benzenesulfonic acid, 2-naphthalenesulfonic acid, 1,5-naphthalenedisulfonic acid, 2-, 3- or 4-methylbenzenesulfonic acid, methylsulfuric acid, ethylsulfuric acid, dodecylsulfuric acid, N-cyclohexylsulfamic acid, N-methyl-, N-ethyl- or N-propyl-sulfamic acid, or other organic protonic acids, such as ascorbic acid.

In the presence of negatively charged radicals, such as carboxy or sulfo, salts may also be formed with bases, e.g. metal or ammonium salts, such as alkali metal or alkaline earth metal salts, for example sodium, potassium, magnesium or calcium salts, or ammonium salts with ammonia or suitable organic amines, such as tertiary monoamines, for example triethylamine or tri(2-hydroxyethyl)amine, or heterocyclic bases, for example N-ethyl-piperidine or N,N′-dimethylpiperazine.

When a basic group and an acid group are present in the same molecule, a compound of formula (I) may also form internal salts.

For isolation or purification purposes it is also possible to use pharmaceutically unacceptable salts, for example picrates or perchlorates. For therapeutic use, only pharmaceutically acceptable salts or free compounds are employed (where applicable in the form of pharmaceutical preparations), and these are therefore preferred.

In view of the close relationship between the compounds in free form and those in the form of their salts, including those salts that can be used as intermediates, for example in the purification or identification of the compounds, tautomers or tautomeric mixtures and their salts, any reference to the compounds hereinbefore and hereinafter especially the compounds of the formula I, is to be understood as referring also to the corresponding tautomers of these compounds, especially of compounds of the formula I, tautomeric mixtures of these compounds, especially of compounds of the formula I, or salts of any of these, as appropriate and expedient and if not mentioned otherwise.

Where “a compound . . . , a tautomer thereof; or a salt thereof” or the like is mentioned, this means “a compound . . . , a tautomer thereof, or a salt of the compound or the tautomer”.

Any asymmetric carbon atom may be present in the (R)—, (S)— or (R,S)-configuration, preferably in the (R)— or (S)-configuration. Substituents at a ring at atoms with saturated bonds may, if possible, be present in cis- (=Z-) or trans (=E-) form. The compounds may thus be present as mixtures of isomers or preferably as pure isomers, preferably as enantiomer-pure diastereomers or pure enantiomers.

The present invention also relates to pro-drugs of a compound of formula (I) that convert in vivo to the compound of formula (I) as such. Any reference to a compound of formula (I) is therefore to be understood as referring also to the corresponding pro-drugs of the compound of formula (I), as appropriate and expedient.

The compounds of formula (I) have valuable pharmacological properties and are useful in the treatment of kinase dependent diseases, e.g., as drugs to treat proliferative diseases.

The term “treatment of tyrosine protein kinase dependent diseases” refers to the prophylactic or preferably therapeutic (including palliative and/or curing) treatment of said diseases, especially of the diseases mentioned below.

Where subsequently the term “USE” is mentioned, this includes any one or more of the following embodiments of the invention, respectively: the use in the treatment of (especially tyrosine) protein kinase dependent diseases, the use for the manufacture of pharmaceutical compositions for use in the treatment of said diseases, methods of use of pyrazolo[1,5a]pyrimidin-7-yl amine derivatives in the treatment of said diseases, pharmaceutical preparations comprising pyrazolo[1,5a]pyrimidin-7-yl amine derivatives for the treatment of said diseases, and pyrazolo[1,5a]pyrimidin-7-yl amine derivatives for use in the treatment of said diseases, as appropriate and expedient, if not stated otherwise. In particular, diseases to be treated and are thus preferred for USE of a compound of formula (I) are selected from (especially tyrosine) protein kinase dependent (“dependent” meaning also “supported”, not only “solely dependent”) diseases mentioned below, especially corresponding proliferative diseases, more especially diseases that depend on c-Abl, Bcr-Abl, c-Kit, c-Raf, Fit-1, Fit-3, KDR, Her-1, PDGFR-kinase, c-Src, RET-receptor kinase, FGF-R1, FGF-R2, FGF-R3, FGF-R4, Ephrin receptor kinases (e.g., EphB2 kinase, EphB4 kinase and related Eph kinases), casein kinases (CK-1, CK-2, G-CK), Pak, ALK, ZAP70, Jak1, Jak2, Axl, Cdk1, cdk4, cdk5, Met, FAK, Pyk2, Syk, Insulin receptor kinase, Tie-2 or constitutively activating mutations of kinases (activating kinases) such as of Bcr-Abl, c-Kit, c-Raf, Flt-3, FGF-R3, PDGF-receptors, RET, and Met, (hereinafter “said kinases”) can therefore be used in the treatment of kinase dependent diseases, especially diseases depending on said kinases and (especially aberrantly highly-expressed or constitutively activated) said kinase-dependent disease or disease dependent on the activation of the said kinase pathways or any combination of two or more of the mentioned kinases.

Most preferred is use of a compound of formula (I) for treating diseases dependant upon c-abl, Flt-3, KDR, c-Src, RET, EphB4, c-kit, cdk1, FGFR-1, c-raf, Her-1, Ins-R and Tek, and use of a compound of formula (I) as an inhibitor of c-abl, Flt-3, KDR, c-Src, RET, EphB4, c-kit, FGFR-1, c-raf, cdk1, Her-1, Ins-R and Tek.

There are also experiments to demonstrate the antitumor activity of compounds of the formula (I) in vivo.

The compounds of formula (I) have valuable pharmacological properties and are useful in the treatment of protein kinase dependent diseases, e.g., as drugs to treat proliferative diseases.

The inhibition of RET is measured as follows: The baculovirus donor vector pFB-GSTX3 is used to generate a recombinant baculovirus that expresses the amino acid region 658-1072 (Swiss prot No. Q9BTB0) of the intra-cytoplasmic kinase domain of human RET-Men2A which corresponds to the wild-type kinase domain of RET (wtRET) and RET-Men2B, which differs from the wtRET by the activating mutation in the activation loop M918T. The coding sequences for the cytoplasmic domain of wtRET and RET-Men2B are amplified by PCR from the plasmids pBABEpuro RET-Men2A and pBABEpuro RET-Men2B. The amplified DNA fragments and the pFB-GSTX3 vector are made compatible for ligation by digestion with Sall and Kpnl. Ligation of these DNA fragments results in the baculovirus donor plasmid pFB-GX3-RET-Men2A and pFB-GX3-RET-Men2B, respectively.

Production of virus: Transfer vectors containing the kinase domains are transfected into the DH10Bac cell line (GIBCO) and plated on selective agar plates. Colonies without insertion of the fusion sequence into the viral genome (carried by the bacteria) are blue. Single, white colonies are picked and viral DNA (bacmid) are isolated from the bacteria by standard plasmid purification procedures. Sf9 cells or Sf21 (American Type Culture Collection) cells are then transfected in 25 cm² flasks with the viral DNA using Cellfectin reagent.

Determination of small scale protein expression in Sf9 cells: Virus-containing media is collected from the transfected cell culture and used for infection to increase its titer. Virus-containing media obtained after two rounds of infection is used for large-scale protein expression. For large-scale protein expression 100 cm² round tissue culture plates are seeded with 5×10⁷ cells/plate and infected with 1 mL of virus-containing media (approximately 5 MOIs). After 3 days, the cells are scraped off the plate and centrifuged at 500 rpm for 5 minutes. Cell pellets from 10-20, 100 cm² plates, are re-suspended in 50 mL of ice-cold lysis buffer (25 mM tris-HCl, pH 7.5, 2 mM EDTA, 1% NP-40, 1 mM DTT, 1 mM P MSF). The cells are stirred on ice for 15 minutes and then centrifuged at 5,000 rpms for 20 minutes.

Purification of GST-tagged proteins: The centrifuged cell lysate is loaded onto a 2 mL glutathione-sepharose column (Pharmacia) and is washed 3× with 10 mL of 25 mM tris-HCl, pH 7.5, 2 mM EDTA, 1 mM DTT, 200 mM NaCl. The GST-tagged proteins are then eluted by 10 applications (1 mL each) of 25 mM tris-HCl, pH 7.5, 10 mM reduced-glutathione, 100 mM NaCl, 1 mM DTT, 10% glycerol and stored at −70° C.

Measure of enzyme activity: Tyrosine protein kinase assays with either purified GST-wtRET or GST-RET-Men2B protein are carried out in a final volume of 30 μL containing 15 ng of either GST-wtRET or GST-RET-Men2B protein, 20 mM tris-HCl, pH 7.5, 1 mM MnCl2, 10 mM MgCl2, 1 mM DTT, 3 μg/mL poly(Glu,Tyr) 4:1, 1% DMSO, 2.0 μM ATP (γ-[³³P]-ATP 0.1 μCi). The activity is assayed in the presence or absence of inhibitors, by measuring the incorporation of ³³P from [γ³³P] ATP into poly(Glu,Tyr) 4:1. The assay is carried out in 96-well plates at ambient temperature for 15 minutes under conditions described below and terminated by the addition of 20 μL of 125 mM EDTA. Subsequently, 40 μL of the reaction mixture are transferred onto Immobilon-PVDF membrane (Millipore) previously soaked for 5 minutes with methanol, rinsed with water, then soaked for 5 minutes with 0.5% H₃PO₄ and mounted on vacuum manifold with disconnected vacuum source. After spotting all samples, vacuum is connected and each well-rinsed with 200 μL 0.5% H₃PO₄. Membranes are removed and washed 4× on a shaker with 1.0% H₃PO₄, once with ethanol. Membranes are counted after drying at ambient temperature, mounting in Packard TopCount 96-well frame, and addition of 10 μL/well of Microscint™ (Packard). IC₅₀ values are calculated by linear regression analysis of the percentage inhibition of each compound in duplicate, at 4 concentrations (usually 0.01, 0.1, 1 and 10 μM). One unit of protein kinase activity is defined as 1 nmole of ³³P ATP transferred from [γ³³P] ATP to the substrate protein/minute/mg of protein at 37° C.

IC₅₀ calculations
input 3 × 4 μL stopped assay on Immobilon membrane, not washed

| | |
|---|---|
| background (3 wells) | assay with H₂O instead of enzyme |
| positive control (4 wells) | 3% DMSO instead of compound |
| bath control (1 well) | no reaction mix |

IC₅₀ values are calculated by logarithmic regression analysis of the percentage inhibition of each compound at 4 concentrations (usually 3- or 10-fold dilution series starting at 10 μM).

In each experiment, the actual inhibition by reference compound is used for normalization of IC₅₀ values to the basis of an average value of the reference inhibitor:
Normalized IC₅₀=measured IC₅₀ average ref. IC₅₀/measured ref. IC₅₀

### EXAMPLE

### Reference Inhibitor in Experiment 0.4 μM, Average 0.3 μM

### Test Compound in Experiment 1.0 μM, Normalization: 0.3/0.4 0.75 μM

For example, staurosporine or a synthetic staurosporine derivative are used as reference compounds.

Using this protocol, the compounds of the formula (I) are found to show IC₅₀ values for RET inhibition in the range from 0.005-100 μM, preferably in the range from 0.01-2 μM.

The efficacy of the compounds of the invention as inhibitors of c-Abl protein-tyrosine kinase activity can be demonstrated as follows: An in vitro enzyme assay is performed in 96-well plates as a filter binding assay as described by Geissler et al. in Cancer Res. 1992; 52:4492-4498, with the following modifications. The His-tagged kinase domain of c-Abl is cloned and expressed in the baculovirus/Sf9 system as described by Bhat et al. in J.Biol.Chem. 1997; 272:16170-16175. A protein of 37 kD (c-Abl kinase) is purified by a two-step procedure over a Cobalt metal chelate column followed by an anion exchange column with a yield of 1-2 mg/L of Sf9 cells (Bhat et al., reference cited). The purity of the c-Abl kinase is >90% as judged by SDS-PAGE after Coomassie blue staining. The assay contains (total volume of 30 μL): c-Abl kinase (50 ng), 20 mM Tris-HCl, pH 7.5, 10 mM MgCl₂, 10 μM Na₃VO₄, 1 mM DTT and 0.06 μCi/assay [γ³³ P]-ATP (5 μM ATP) using 30 μg/mL poly-Ala,Glu,Lys,Tyr-6:2:5:1 (Poly-AEKY, Sigma P1152) in the presence of 1% DMSO. Reactions are terminated by adding 10 μL of 250 mM EDTA and 30 μL of the reaction mixture is transferred onto Immobilon-PVDF membrane (Millipore, Bedford, Mass., USA) previously soaked for 5 min with methanol, rinsed with water, then soaked for 5 min with 0.5% H₃PO₄ and mounted on vacuum manifold with disconnected vacuum source. After spotting all samples, vacuum is connected and each well rinsed with 200 μL 0.5 % H₃PO₄. Membranes are removed and washed on a shaker with 0.5% H₃PO₄ (4 times) and once with ethanol. Membranes are counted after drying at ambient temperature, mounting in Packard TopCount 96-well frame, and addition of 10 μL/well of Microscint™ (Packard).

Using this test system, compounds of the formula I show lC₅₀ values of inhibition for c-Abl inhibition in the range of 0.002 to 100 μM, usually between 0.002 and 5 μM.

The efficacy of the compounds of the invention as inhibitors of KDR protein-tyrosine kinase activity can be demonstrated as follows: The inhibition of VEGF-induced receptor autophosphorylation can be confirmed with a further in vitro experiments in cells such as transfected CHO cells, which permanently express human VEGF-R2 receptor (KDR), are seeded in complete culture medium (with 10% fetal calf serum=FCS) in 6-well cell-culture plates and incubated at 37° C. under 5% CO₂ until they show about 80% confluency. The compounds to be tested are then diluted in culture medium (without FCS, with 0.1% bovine serum albumin) and added to the cells. (Controls comprise medium without test compounds). After two hours' incubation at 37° C., recombinant VEGF is added; the final VEGF concentration is 20 ng/ml). After a further five minutes incubation at 37° C., the cells are washed twice with ice-cold PBS (phosphate-buffered saline) and immediately lysed in 100 μl lysis buffer per well. The lysates are then centrifuged to remove the cell nuclei, and the protein concentrations of the supernatants are determined using a commercial protein assay (BIORAD). The lysates can then either be immediately used or, if necessary, stored at −20° C.

A sandwich ELISA is carried out to measure the VEGF-R2 phosphorylation: a monoclonal antibody to VEGF-R2 (for example Mab 1495.12.14; ProQinase, Freiburg, Germany) is immobilized on black ELISA plates (OptiPlate™ HTRF-96 from Packard). The plates are then washed and the remaining free protein-binding sites are saturated with 3% TopBlock® (Juro, Cat. # TB232010) in phosphate buffered saline with Tween 20® (polyoxyethylen(20)-sorbitane monolaurate, ICI/Uniquema)-(PBST). The cell lysates (20 μg protein per well) are then incubated in these plates overnight at 4° C. together with an antiphosphotyrosine antibody coupled with alkaline phosphatase (PY20:AP from Zymed). The (plates are washed again and the) binding of the antiphosphotyrosine antibody to the captured phosphorylated receptor is then demonstrated using a luminescent AP substrate (CDP-Star, ready to use, with Emerald II; Applied Biosystems). The luminescence is measured in a Packard Top Count Microplate Scintillation Counter. The difference between the signal of the positive control (stimulated with VEGF) and that of the negative control (not stimulated with VEGF) corresponds to VEGF-induced VEGF-R2 phosphorylation (=100%). The activity of the tested substances is calculated as percent inhibition of VEGF-induced VEGF-R2 phosphorylation, wherein the concentration of substance that induces half the maximum inhibition is defined as the IC₅₀ (inhibitory dose for 50% inhibition). Compounds of the formula I here show an IC₅₀ in the range of 0.005 to 20 μM, preferably between 0.005 and 1 μM for KDR inhibition.

Flt3 kinase inhibition is determined as follows: The baculovirus donor vector pFbacG01 (GIBCO) is used to generate a recombinant baculovirus expressing the amino acid region amino acids 563-993 of the cytoplasmic kinase domain of human Flt-3. The coding sequence for the cytoplasmic domain of Flt-3 is amplified by PCR from human c-DNA libraries (Clontech). The amplified DNA fragments and the pFbacG01 vector are made compatible for ligation by digestion with BamH1 and HindIII. Ligation of these DNA fragments results in the baculovirus donor plasmid Flt-3(1.1). The production of the viruses, the expression of proteins in Sf9 cells and the purification of the GST-fused proteins are performed as follows:

Production of virus: Transfer vector (pFbacG01-Flt-3) containing the Flt-3 kinase domain is transfected into the DH10Bac cell line (GIBCO) and the transfected cells are plated on selective agar plates. Colonies without insertion of the fusion sequence into the viral genome (carried by the bacteria) are blue. Single white colonies are picked and viral DNA (bacmid) is isolated from the bacteria by standard plasmid purification procedures. Sf9 or Sf21 cells (American Type Culture Collection) are then transfected in flasks with the viral DNA using Cellfectin reagent.

Determination of small scale protein expression in Sf9 cells: Virus containing media is collected from the transfected cell culture and used for infection to increase its titre. Virus containing media obtained after two rounds of infection is used for large-scale protein expression. For large-scale protein expression 100 cm² round tissue culture plates are seeded with 5×10cells/plate and infected with 1 mL of virus-containing media (approx. 5 MOIs). After 3 days the cells are scraped off the plate and centrifuged at 500 rpm for 5 min. Cell pellets from 10-20, 100 cm² plates, are resuspended in 50 mL of ice-cold lysis buffer (25 mM Tris-HCl, pH 7.5, 2mM EDTA, 1% NP-40, 1 mM DTT, 1 mM PMSF). The cells are stirred on ice for 15 min and then centrifuged at 5000 rpms for 20 min.

Purification of GST-tagged proteins: The centrifuged cell lysate is loaded onto a 2 mL glutathione-sepharose column (Pharmacia) and washed three times with 10 mL of 25 mM Tris-HCl, pH 7.5, 2 mM EDTA, 1 mM DTT, 200 mM NaCl. The GST-tagged protein is then eluted by 10 applications (1 mL each) of 25 mM Tris-HCl, pH 7.5, 10 mM reduced-glutathione, 100 mM NaCl, 1 mM DTT, 10% Glycerol and stored at −70° C.

Measurement of enzyme activity: Tyrosine protein kinase assays with purified GST-Flt-3 are carried out in a final volume of 30 μL containing 200-1800 ng of enzyme protein (depending on the specific activity), 20 mM Tris-HCl, pH 7.6, 3 mM MnCl₂, 3 mM MgCl₂, 1 mM DTT, 10 μM Na₃VO₄, 3 μg/mL poly(Glu,Tyr) 4:1, 1% DMSO, 8.0 μM ATP and 0.1 μCi [γ³³P] ATP). The activity is assayed in the presence or absence of inhibitors, by measuring the incorporation of ³³P from [γ³³P] ATP into the poly(Glu,Tyr) substrate. The assay (30 μL) is carried out in 96-well plates at ambient temperature for 20 min under conditions described below and terminated by the addition of 20 μL of 125 mM EDTA. Subsequently, 40 μL of the reaction mixture is transferred onto Immobilon-PVDF membrane (Millipore, Bedford, Mass., USA) previously soaked for 5 min with methanol, rinsed with water, then soaked for 5 min with 0.5% H₃PO₄ and mounted on vacuum manifold with disconnected vacuum source. After spotting all samples, vacuum is connected and each well rinsed with 200 μL 0.5% H₃PO₄. Membranes are removed and washed 4× on a shaker with 1.0% H₃PO₄, once with ethanol. Membranes are counted after drying at ambient temperature, mounting in Packard TopCount 96-well frame, and addition of 10 μL/well of Microscint™ (Packard). IC₅₀ values are calculated by linear regression analysis of the percentage inhibition of each compound in duplicate, at four concentrations (usually 0.01, 0.1, 1 and 10 μM). One unit of protein kinase activity is defined as 1 nmole of ³³P ATP transferred from [γ³³P] ATP to the substrate protein per minute per mg of protein at 37° C. The compounds of the formula I show IC₅₀ values for Flt-3 inhibition in the range between 0.01 and 100 μM, preferably between 0.05 and 10 μM.

The compounds of formula I also inhibit other tyrosine protein kinases such as especially the c-Src kinase, c-Kit, VEGF-R and/or FGFR; all of which play a part in growth regulation and transformation in animal, especially mammal cells, including human cells. An appropriate assay is described in Andrejauskas-Buchdunger et al., Cancer Res. 52, 5353-8 (1992). Using this test system, compounds of the formula I show IC₆₀ values for inhibition of c-Src in the range of 0.005 to 100 μM, usually between 0.005 and 5 μM. Compounds of formula I also show IC₅₀ values for c-kit inhibition in the range of 0.005 to 10 μM, usually between 0.005 and 5 μM; and for inhibition of FGFR-1, up to 95% inhibition at 10 μM.

The inhibition of IGF-1 R and Ins-R can be determined as follows: The baculovirus donor vector pfbgx3IGFIRcd is used to generate a recombinant baculovirus that expresses the amino acid region 950-1337 of the mature peptide cytoplasmic domain of the human IGF-IR. To generate the cDNA fragment encoding the amino acid region 919-1343 of the intra-cytoplasmic kinase domain of the human insulin receptor, pC5hInsR is used. The fragments of the human IGF-IR and Ins-R are cloned, expressed and small-scale purified as a factor Xa-cleavable glutathione-S-transferase (GST)-fusion protein using the Bac-to-Bac™ system (GIBCO BRL) of recombinant baculovirus generation. Virus containing media iss collected from the transfected cell culture and used for infection to increase its titer. Virus containing media obtained after two rounds of infection iss used for large-scale protein expression. Cell extracts are prepared and loaded onto a glutathione-Sepharose (Pharmacia) column. After washing, the GST-tagged proteins are then eluted with a glutathione-containing buffer. Purified protein is stored at −70° C. in elution buffer. Tyrosine protein kinase assays with purified GST-IGF-1R and GST-Ins-R are carried in a final volume of 30 μl containing 20 mM Tris-HCl, pH 7.6, 10 mM MgCl₂, 0.01 mM Na₃VO₄, 1% DMSO, 1 mM DTT, 3 μg/ml poly(Glu,Tyr) 4:1 and 10 μM ATP (γ-[³³P]-ATP 0.1 μCi). The assay is performed in 96-well plates at ambient temperature for 20 min and terminated by addition of 25 μl 0.05 M EDTA pH 7.0. An aliquot of 40 μl is spotted with a multichannel dispenser on Whatman P81 membranes mounted in a Millipore Microtiter filter manifold connected to a low vacuum source. After elimination of liquid, the membrane is transferred to a sequence of 4 washing baths containing 0.5% H₃PO₄ and one with EtOH (shaking incubation for 10 min each), dried, mounted onto a Hewlett Packard TopCount manifold added 10 μl Microscint® and counted. Compounds of formula (I) show up to 90% inhibition of Ins-R at 10,000 nM, preferably between 60-90% inhibition.

The inhibition of Tek can be determined as follows: The procedure of the expression, purification and assay these kinases has been described. Fabbro et al., Pharmacol. Ther. 82(2-3) 293-301 (1999). In brief, the glutathione S-transferase (GST) gene from the pAcG1 vector (Pharmingen) is excised with EcoRV and EcoRI and inserted into the cloning site of the Fast-Bac baculoviral vector (GIBCO) creating a 5530 bp vector with N-terminal cloning sites derived from the pAcG1 fusion vector (FBG0). The C-terminal cloning site may be any cloning site (from the Fast-Bac vector) downstream of the N-terminal cloning site used. N-terminally GST-fused (pAcG1, Pharmingen) KDR, Fit-1, Flk-1, Tek and PDGFR-P kinase domains are obtained from ProQinase, Freiburg, Germany. Tek is recloned into the FBG1 vector by EcoRI excision and ligation into EcoRI digested FBG1 (FBG1-Tek). The coding sequences for the whole cytoplasmic domain of c-Kit (aa 544-976) and c-Fms (aa 538-972) are amplified by PCR from human uterus and from human bone marrow cDNA libraries (Clontech), respectively. The amplified DNA fragments are fused to GST by cloning them into FBG1 as BamHI-EcoRI insertions, to yield FBG1-c-Kit and FBG1-c-Fms. Tek is recloned into the FBG0 transfer vector by EcoRI excision and ligation into EcoRI digested FBG0 (FBG-Tie2/Tek). FGFR-1 and c-met kinase domains are obtained by PCR from human A431 cells. N-terminal primers contain an overhanging EcoRI site, while C-terminal primers contain a XhoI site to aid cloning into the transfer vectors. After digestion of both the PCR fragments and FBG0 the cleavage products are gel-purified and ligated together to form the kinase constructs (FBG-Met, FBG-FGFR-1).

Viruses for each of the kinases are made according to the protocol supplied by GIBCO. In brief, transfer vectors containing the kinase domains are transfected into the DH10Bac cell line (GIBCO), plated on agar plates containing the recommended concentrations of Blue-Gal, IPTG, Kanamycin, Tetracycline, and Gentamycin. Colonies without insertion of the fusion sequence into the viral genome (carried by the bacteria) are blue. A single white colony is usually picked and viral DNA (bacmid) isolated from the bacteria by standard plasmid mini prep procedures. Sf9 cells or High Five cells (GIBCO) are then transfected in 25 cm² flasks with the viral DNA using the Cellfectin reagent and protocol supplied with the Bac-to-Bac kit (GIBCO). Virus containing media is collected from the transfected cell culture and used for infection to increase its titer. Virus containing media obtained after two rounds of infection is used for large-scale protein expression. For large-scale protein expression 100 cm² round tissue culture plates are seeded with 5×10⁷ cells/plate and infected with 1 ml of virus-containing media (about 5 MOIs). After 3 days the cells are scraped off the plate and centrifuged at 500 rpm for 5 min.

Cell pellets from 10-20,100 cm² plates, are resuspended in 50 ml of ice-cold lysis buffer (25 mM Tris-HCl, pH 7.5, 2 mM EDTA, 1% NP-40, 1 mM DTT, 1 mM PMSF). The cells are stirred on ice for 15 min and then centrifuged at 5000 rpms for 20 min. The supernatant is loaded onto a 2 ml glutathione-sepharose column and washed three times with 10 ml of 25 mM Tris-HCl, pH 7.5, 2 mM EDTA, 1 mM DTT, 200 mM NaCl. The GST-tagged proteins are then eluted by 10 applications (1 ml each) of 25 mM Tris-HCl, pH 7.5, 10 mM reduced-glutathione, 100 mM NaCl, 1 mM DTT, 10% Glycerol and stored at −70° C.

The assays (30 μl) contain 200-1800 ng of enzyme protein (depending on the specific activity), 20 mM Tris-HCl, pH 7.6, 3 mM MnCl₂, 3 mM MgCl₂, 1 mM DTT, 10 μM Na₃VO₄, 3 μg/ml poly(Glu,Tyr) 4:1, 8 μM ATP (γ-[³³P]-ATP 0.1 μCi). Reactions are incubated for 20 min at ambient temperature and then stopped by addition of 25 μl 0.25 M EDTA (pH 7.0). An aliquot of 40 μl is spotted with a multichannel dispenser on Immobilon P membranes mounted in a Millipore Microtiter filter manifold connected to a low vacuum source. After elimination of liquid, the membrane is transferred to a sequence of 4 washing baths containing 0.5% H₃PO₄ and one with EtOH (shaking incubation for 10 min each), dried, mounted onto a Hewlett Packard TopCount manifold added 10 μl Microscint® and counted. Compounds of formula (I) show IC₅₀ values, calculated by linear regression analysis, for Tek inhibition of about 0.1-100 μM.

The inhibition of Cdk1 can be determined as follows: Cdk1/cycB: Cdk1/cycB are obtained from ProQinase, Freiburg, Germany. Starfish oocytes are induced to enter M phase of the cell cycle with 10 μM 1-methyladenine and frozen in liquid nitrogen and stored at −80° C. When required, the oocytes are homogenized and centrifuged as described (Arion et al., Cell 55:371-378 (1988) and Rialet et al., Anticancer Res. 11: 1581-1590 (1991)). Cdk1/cycB kinase is purified on p9^{CKShs}-sepharose beads and eluted with recombinant human p9^{CKShs} as described (Azzi et al., Eur. J. Biochem. 203: 353-360. (1992)). Briefly, the supernatant from oocytes is equilibrated for 30 min at 4° C. under constant rotation with the p9^{CKShs}-sepharose beads. The beads are extensively washed and active cdk1/cycB kinase is eluted with purified p9^{CKShs} (3 mg/ml). The activity of Cdk1/cycB is measured as described (Arion et al., Cell 55: 371-378 (1988), Meijer et al., EMBO J. 1989; 8: 2275-2282 and Meijer et al., EMBO J. 1991; 8: 2275-2282). The assay is carried with slight modifications in 96-well plates at ambient temperature for 20 min. The final volume of 30 μl contains 0.1-0.3U of Cdk1/cycB, 1 mg/ml histone H1 as a substrate, 60 mM β-glycerophosphate, 30 mM nitrophenylphosphate, 25 mM MOPS, 5 mM EGTA, 15 mM MgCl₂, 1 mM DTT, 0.1 mM Na₃VO₄, 15 μM ATP and 0.1 μCi γ-³³P-ATP (75 μM, 8800 cpm/pmole). The reaction is terminated by addition of 25 μl 0.05 M EDTA pH 7.0. An aliquot of 40 μl is spotted with a multichannel dispenser on Immobilon P membranes mounted in a Millipore Microtiter filter manifold connected to a low vacuum source. After elimination of liquid, the membrane is transferred to a sequence of 4 washing baths containing 0.5% H₃PO₄ and one with EtOH (shaking incubation for 10 min each), dried, mounted onto a Hewlett Packard TopCount manifold added 10 μl Microscint® and counted. Compounds of formula (I) show up to 100% Cdk1 inhibition at 10,000 nM.

The inhibition of c-Raf-1 can be determined as follows: Production of recombinant c-Raf-1 protein, is obtained by triple infection of Sf21 cells with GST-c-Raf-1 recombinant baculovirus together with v-Src and v-Ras recombinant baculoviruses that are required for active c-Raf-1 kinase production (Williams et al., PNAS 1992; 89: 2922-2926). Active Ras (v-Ras) is required to recruit c-Raf-1 to the cell membrane and v-Src to phosphorylate c-Raf-1 to fully activate it (Williams et al., PNAS 1992; 89: 2922-2926). Cells were seeded at 2.5×10⁷ cells per 150 mm dish and allowed to attach to a 150 mm dish for 1 hr at RT. Media (SF90011 containing 10% FBS) is aspirated and recombinant baculovirus; GST-C-Raf-1, v-Ras and v-Src are added at MOI of 3.0, 2.5 and 2.5 receptively in a total volume of 4-5 mL. Cells are incubated for 1 hr at RT and then 15 mL of medium is added. Infected cells are incubated for 48-72 hr at 27° C. Infected Sf21 cells are scraped and collected into a 50 mL tube and centrifuged for 10 min at 4° C. at 1100 g in a Sorvall centrifuge. The cell pellet is washed once with ice cold PBS and lysed with 0.6 mL lysis buffer per 2.5×10⁷ cells. Complete lysis of cells is achieved after 10 min on ice with occasional pipetting. The cell lysates are centrifuged for 10 min at 4° C. at 14,500 g in a Sorvall centrifuge with SS-34 rotor and the supernatant is transferred to a fresh tube and stored at −80° C. c-Raf-1 is purified from cell lysates using 100 uL of packed Glutathione-Sepharose 4B beads equilibrated in ice cold PBS per 2.5×10⁷ cells. GST-c-Raf-1 was allowed to bind to the beads at 4° C. for 1 hr with rocking. Bound GST-c-Raf-1 with beads was transferred to a column. The column is washed once with lysis buffer and twice with ice cold Tris buffered saline. Ice cold elution buffer is added and column flow is stopped to allow the free glutathione to disrupt the interaction of GST-c-Raf-1 with glutathione sepharose beads. Fractions (1 mL) are collected into pre-chilled tubes. Each tube contains 10% glycerol (final concentration) to maintain kinase activity during freeze thaw cycles. Purified fractions of GST-c-Raf-1 kinase protein are stored at −80° C.

IκB was used as substrate for the c-Raf-1 kinase. IκB is expressed in bacteria as a His-tagged protein (cloned and kindly provided by Dr. Eder; ABM, Novartis, Basel). BL21 LysS bacteria containing the IκB plasmid are grown to an OD₆₀₀ of 0.6 in LB medium then induced to express the kb with IPTG (final concentration of 1 mM) for 3 hrs at 37° C. and then bacteria are lysed by sonication (microtip limit setting for 3 times at 1 min each in sonication buffer [50 mM Tris pH 8.0, 1 mM DTT, 1 mM EDTA] and centrifuged at 10,000 g for 15 min. The supernatant is mixed with ammonium sulfate to give a final concentration of 30%. This mixture is rocked for 15 min at 4° C. then spun at 10,000 g for 15 min. The pellet is resuspended in binding buffer (Novagen) containing 10 mM BSA. This solution is applied to Ni-agarose (Novagen) and washed according to the Novagen manual. IκB is eluted from the column using elution buffer (0.4 M imidazole, 0.2 M NaCl, 8 mM Tris pH 7.9). Fractions containing protein are dialysed in 50 mM Tris pH 8, 1 mM DTT.

The activity of c-Raf-1 protein kinase is assayed in the presence or absence of inhibitors, by measuring the incorporation of ³³P from [γ³³P] ATP into IB. The assay is carried out in 96-well plates at ambient temperature for 60 min. It contains (total volume of 30 μl): c-rafl1 kinase (400 ng), 25 mM Tris-HCl, pH 7.5, 5 mM MgCl₂, 5 mM MnCl₂, 10 μM Na₃VO₄, 1 mM DTT and 0.3 μCi/assay [γ³³P]-ATP (10 μM ATP) using 600 ng IB in the presence of 1% DMSO. Reactions are terminated by adding 10 μL of 250 mM EDTA and 30 μL of the reaction mixture is transferred onto Immobilon-PVDF membrane (Millipore, Bedford, Mass., USA) previously soaked for 5 min with methanol, rinsed with water, then soaked for 5 min with 0.5% H₃PO₄ and mounted on vacuum manifold with disconnected vacuum source. After spotting all samples, vacuum is connected and each well rinsed with 200 μL 0.5% H₃PO₄. Membranes are removed and washed 4× on a shaker with 0.5% H₃PO₄, once with ethanol. Membranes are counted after drying at ambient temperature, mounting in Packard TopCount 96-well frame, and addition of 10 μL/well of Microscint™ (Packard).

Compounds of formula (I) show c-Raf-1 inhibition in the range between 0.1-50 μM, preferably between 0.1 and 10 μM.

The (especially important and preferred) efficacy of compounds of the formula I as inhibitors or Ephrin B4 receptor (EphB4) kinases can be demonstrated as follows:

EphB4: Production and measure of enzymatic activity: Generation of Bac-to-Bac™ (Initrogen Life Technologies, Basel, Switzerland) GST-fusion expression vectors: Entire cytoplasmatic coding regions of the EphB-class are amplified by PCR from cDNA libraries derived from human placenta or brain, respectively. Recombinant baculovirus are generated that express the amino acid region 566-987 of the human EphB4 receptor (SwissProt Database, Accession No. P54760). GST sequence is cloned into pFastBac1® vector (Invitrogen Life Technologies, Basel, Switzerland) and PCR amplified. cDNAs encoding EphB4-receptor domains, respectively, are cloned in frame 3′ prime to the GST sequence into this modified FastBac1 vector to generate pBac-to-Bac™ donor vectors. Single colonies arising from the transformation are inoculated to give overnight cultures for small scale plasmid preparation. Restriction enzyme analysis of plasmid DNA reveals several clones to contain inserts of the expected size. By automated sequencing the inserts and approximately 50 bp of the flanking vector sequences are confirmed on both strands.

Production of viruses: Viruses for each of the kinases are made according to the protocol supplied by GIBCO if not stated otherwise. In brief, transfer vectors containing the kinase domains are transfected into the DH10Bac cell line (GIBCO) and plated on selective agar plates. Colonies without insertion of the fusion sequence into the viral genome (carried by the bacteria) are blue. Single white colonies are picked and viral DNA (bacmid) isolated from the bacteria by standard plasmid purification procedures. Sf9 cells or Sf21 cells are then transfected in 25 cm2 flasks with the viral DNA using Cellfectin reagent according to the protocol.

Purification of GST-tagged kinases: The centrifuged cell lysate is loaded onto a 2 mL glutathione-sepharose column (Pharmacia) and washed three times with 10 mL of 25 mM Tris-HCl, pH 7.5, 2 mM EDTA, 1 mM DTT, 200 mM NaCl. The GST-tagged proteins are then eluted by 10 applications (1 mL each) of 25 mM Tris-HCl, pH 7.5, 10 mM reduced-glutathione, 100 mM NaCl, 1 mM DTT, 10% Glycerol and stored at −70° C.

Protein kinase assays: The activities of protein kinases are assayed in the presence or absence of inhibitors, by measuring the incorporation of 33P from [γ33P]ATP into a polymer of glutamic acid and tyrosine (poly(Glu,Tyr)) as a substrate. The kinase assays with purified GST-EphB (30 ng) are carried out for 15-30 min at ambient temperature in a final volume of 30 μL containing 20 mM Tris.HCl, pH 7.5,10 mM MgCl2, 3-50 mM MnCl2, 0.01 mM Na3VO4, 1% DMSO, 1 mM DTT, 3 μg/mL poly(Glu,Tyr) 4:1 (Sigma; St. Louis, Mo., USA) and 2.0-3.0 μM ATP (γ-[33P]-ATP 0.1 μCi). The assay is terminated by the addition of 20 μL of 125 mM EDTA. Subsequently, 40 μl of the reaction mixture are transferred onto Immobilon-PVDF membrane (Millipore, Bedford, Mass., USA) previously soaked for 5 min with methanol, rinsed with water, then soaked for 5 min with 0.5% H3PO4 and mounted on vacuum manifold with disconnected vacuum source. After spotting all samples, vacuum is connected and each well rinsed with 200 μl 0.5% H3PO4. Membranes are removed and washed 4× on a shaker with 1.0% H3PO4, once with ethanol. Membranes are counted after drying at ambient temperature, mounting in Packard TopCount96-well frame, and addition of 10 μL/well of Microscint™ (Packard). IC50 values are calculated by linear regression analysis of the percentage inhibition of each compound in duplicate, at four concentrations (usually 0.01, 0.1, 1 and 10 μM). One unit of protein kinase activity is defined as 1 nmole of 33P ATP transferred from [γ33P] ATP to the substrate protein per minute per mg of protein at 37° C. Compounds of formula I show EphB4 inhibition down to 10 nM, preferably IC50 values between 0.01-1.0 μM.

Cellular Assay: EphB4Receptor Autophosphorylation:

The inhibition of EphB4 receptor autophosphorylation can be confirmed with an in vitro experiment in cells such as transfected A375 human melanoma cells (ATCC Number: CRL-1619), which permanently express human EphB4 (SwissProt AccNo P54760), are seeded in complete culture medium (with 10% fetal calf serum=FCS) in 6-well cell-culture plates and incubated at 37° C. under 5% CO₂ until they show about 90% confluency. The compounds to be tested are then diluted in culture medium (without FCS, with 0.1% bovine serum albumin) and added to the cells. (Controls comprise medium without test compounds). Ligand induced autophosphorylation is induced by the addition of 1 microg/ml soluble ephrinB2-Fc (s-ephrinB2-Fc: R&D Biosystems, CatNr 496-EB) and 0.1 microM ortho-vanadate. After a further 20 minutes incubation at 37° C., the cells are washed twice with ice-cold PBS (phosphate-buffered saline) and immediately lysed in 200 μl lysis buffer per well. The lysates are then centrifuged to remove the cell nuclei, and the protein concentrations of the supernatants are determined using a commercial protein assay (PIERCE). The lysates can then either be immediately used or, if necessary, stored at −20° C.

A sandwich ELISA is carried out to measure the EphB4 phosphorylation: To capture phosphorylated EphB4 protein 100 ng/well of ephrinB2-Fc (s-ephrinB2-Fc: R&D Biosystems, CatNr 496-EB) is immobilized MaxiSorb (Nunc) ELISA plates. The plates are then washed and the remaining free protein-binding sites are saturated with 3% TopBlock® (Juro, Cat. # TB232010) in phosphate buffered saline with Tween 20® (polyoxyethylen(20)sorbitane monolaurate, ICI/Uniquema) (PBST). The cell lysates (100 μg protein per well) are then incubated in these plates for 1 h at room temperature. After washing the wells three times with PBS an antiphosphotyrosine antibody coupled with alkaline phosphatase (PY 20 Alkaline Phosphate conjugated: ZYMED, Cat Nr03-7722) is added and incubated for another hour. The plates are washed again and the binding of the antiphosphotyrosine antibody to the captured phosphorylated receptor is then demonstrated and quantified using 10 mM D-nitrophenylphosphat as subtrate and measuring the OD at 405 nm after 0.5h-1 h. The difference between the signal of the positive control (stimulated with vanadate and s-ephrinB2-Fc) and that of the negative control (not stimulated) corresponds to maximal EphB4 phosphorylation (=100%). The activity of the tested substances is calculated as percent inhibition of maximal EphB4 phosphorylation, wherein the concentration of substance that induces half the maximum inhibition is defined as the IC₅₀ (inhibitory dose for 50% inhibition).

Experiments to demonstrate the antitumor activity of compounds of the formula (I) in vivo: For example, in order to test whether a compound of the formula (I), e.g. that of Example 1 given below, inhibits VEGF-mediated angiogenesis in vivo, its effect on the angiogenic response induced by VEGF in a growth factor imlant model in mice is tested: A porous Teflon chamber (volume 0.5 mL) is filled with 0.8% w/v agar containing heparin (20 units/ml) with or without growth factor (2 μg/ml human VEGF) is implanted subcutaneously on the dorsal flank of C57/C6 mice. The mice are treated with the test compound (e.g. 25, 50 or 100 mg/kg p.o. once daily) or vehicle starting on the day of implantation of the chamber and continuing for 4 days after. At the end of the treatment, the mice are killed, and the chambers are removed. The vascularized tissue growing around the chamber is carefully removed and weighed, and the blood content is assessed by measuring the hemoglobin content of the tissue (Drabkins method; Sigma, Deisenhofen, Germany). It has been shown previously that these growth factors induce dose-dependent increases in weight and blood content of this tissue growing (characterized histologically to contain fibroblasts and small blood vessels) around the chambers and that this response is blocked by antibodies that specifically neutralize VEGF (see Wood J M et al., Cancer Res. 60(8), 2178-2189, (2000); and Schlaeppi et al., J. Cacner Res. Clin. Oncol. 125, 336-342, (1999)). With this model, inhibition can be shown in the case of compounds of the formula (I).

Synthetic Procedure

Compounds of formula (I) are prepared analogously to the procedure described by Alicade, E; De Mendoza, J; Garcia-Marquina, J M; Almera, C; J. Heterocycl. Chem. 11, 423 (1974) by:
(a) reacting a nitrile, A-CH₂—C≡N, with ethyl formate in the presence of an organic solvent to form a substituted 3-oxo-propionitrile,
(b) condensing the substituted 3-oxo-propionitriles of step (a) with hydrazine monohydrate in an organic solvent to form a 2H-pyrazol-3-ylamine of formula (III): 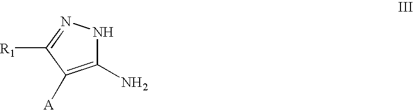
(d) formylating a substituted nitrile in the presence of ethanolate and formic acid ethyl ester to prepare a 3-oxo-propionitrile of formula (II): 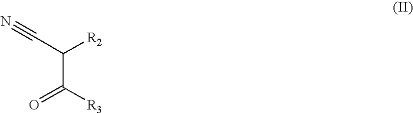
(c) condensing the 3-oxo-propionitrile of formula (II) and the 2H-pyrazol-3-ylamines of formula (III) in the presence of an organic solvent to form a compound of formula (I).

Specifically, compounds of formula (I) are prepared by condensing 3-oxo- propionitriles (II) and the corresponding 2H-pyrazol-3-ylamines (III) in the presence of ethanolic HCl (FIG. 2). The 2H-pyrazol-3-ylamines (III) are prepared by condensing hydrazine monohydrate with the corresponding 3-oxo-propionitriles dissolved in an organic solvent, such as EtOH, dioxane or AcOH and heated at elevated temperatures (preferably at 100° C.) for several hours. The preferred procedure for preparing the pyrazolo moiety of the title compounds was stirring the hydrazine monohydrate with the corresponding 3-oxo-propionitriles in acetic acid at 100° C. for 2-3 h followed by addition of aqueous HCl and further refluxing the reaction mixture for further 20 min. In case where R1 is not H, the corresponding substituted hydrazines are used. The 3-oxo-propionitriles (I) and (II) are synthesized from the corresponding nitriles by classical formylation reaction using freshly prepared sodium ethanolate and formic acid ethyl ester (refluxing for 1 h in EtOH). Alternatively, instead of performing the condensation reactions with the 3-oxo-propionitiles, the corresponding 3,3-dialkoxy-propionitiles (in analogy to the procedure described by Seneci, P., Nicola, M., Inglesi, M., Vanotti, E., Resnati, G. Synth. Commun. 29 (2), 311-341 (1999)) or 3-dimethylamino-acrylonitriles can be used.

[image]

Alternatively, compounds of formula (I) can be prepared by first synthesizing the pyrazolo[1,5-a]pyrimidin-7-ylamine core scaffold carrying a corresponding functional groups (X, see FIG. 3) where residues A, R₂, or R₃, respectively, can be introduced by known reactions as indicated in FIG. 3.

[image]

wherein R₁, R₂, R₃, and X are as defined for compounds of the formula I,

and, if desired, after reaction (a), (b) or (c), transforming an obtainable compound of formula (I) into a different compound of formula l; transforming a salt of an obtainable compound of formula (I) into the free compound or a different salt or an obtainable free compound of formula (I) into a salt; and/or separating an obtainable mixture of isomers of compounds of formula (I) into the individual isomers;
where for all reactions mentioned functional groups in the starting materials that shall not take part in the reaction are, if required, present in protected form by readily removable protecting groups, and any protecting groups are subsequently removed.

The following reaction conditions are preferred, respectively:

Within the scope of this text, only a readily removable group that is not a constituent of the particular desired end product of formula (I) is designated a “protecting group”, unless the context indicates otherwise. The protection of functional groups by such protecting groups, the protecting groups themselves, and their cleavage reactions are described for example in standard reference works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/I, Georg Thieme Verlag, Stuttgart 1974, in H.-D. Jakubke and H. Jeschkeit, “Aminosäuren, Peptide, Proteine” (Amino acids, Peptides, Proteins), Verlag Chemie, Weinheim, Deerfield Beach, and Basel 1982, and in Jochen Lehmann, “Chemie der Kohlenhydrate: Monosaccharide und Derivate” (Chemistry of Carbohydrates: Monosaccharides and Derivatives), Georg Thieme Verlag, Stuttgart 1974. A characteristic of protecting groups is that they can be removed readily (i.e. without the occurrence of undesired secondary reactions) for example by solvolysis, reduction, photolysis or alternatively under physiological conditions (e.g. by enzymatic cleavage).

Salts of compounds of formula (I) having at least one salt-forming group may be prepared in a manner known per se. For example, salts of compounds of formula (I) having acid groups may be formed, for example, by treating the compounds with metal compounds, such as alkali metal salts of suitable organic carboxylic acids, e.g. the sodium salt of 2-ethylhexanoic acid, with organic alkali metal or alkaline earth metal compounds, such as the corresponding hydroxides, carbonates or hydrogen carbonates, such as sodium or potassium hydroxide, carbonate or hydrogen carbonate, with corresponding calcium compounds or with ammonia or a suitable organic amine, stoichiometric amounts or only a small excess of the salt-forming agent preferably being used. Acid addition salts of compounds of formula (I) are obtained in customary manner, e.g. by treating the compounds with an acid or a suitable anion exchange reagent. Internal salts of compounds of formula (I) containing acid and basic salt-forming groups, e.g. a free carboxy group and a free amino group, may be formed, e.g. by the neutralisation of salts, such as acid addition salts, to the isoelectric point, e.g. with weak bases, or by treatment with ion exchangers.

Salts can be converted in customary manner into the free compounds; metal and ammonium salts can be converted, for example, by treatment with suitable acids, and acid addition salts, for example, by treatment with a suitable basic agent.

Mixtures of isomers obtainable according to the invention can be separated in a manner known per se into the individual isomers; diastereoisomers can be separated, for example, by partitioning between polyphasic solvent mixtures, recrystallisation and/or chromatographic separation, for example over silica gel or by e.g. medium pressure liquid chromatography over a reversed phase column, and racemates can be separated, for example, by the formation of salts with optically pure salt-forming reagents and separation of the mixture of diastereoisomers so obtainable, for example by means of fractional crystallisation, or by chromatography over optically active column materials.

Intermediates and final products can be worked up and/or purified according to standard methods, e.g. using chromatographic methods, distribution methods, (re-) crystallization, and the like.

General Process Conditions

The following applies in general to all processes mentioned hereinbefore and hereinafter, while reaction conditions specifically mentioned above or below are preferred:

All the above-mentioned process steps can be carried out under reaction conditions that are known per se, preferably those mentioned specifically, in the absence or, customarily, in the presence of solvents or diluents, preferably solvents or diluents that are inert towards the reagents used and dissolve them, in the absence or presence of catalysts, condensation or neutralizing agents, for example ion exchangers, such as cation exchangers, e.g. in the H⁺ form, depending on the nature of the reaction and/or of the reactants at reduced, normal or elevated temperature, for example in a temperature range of from about −100° C. to about 190° C., preferably from approximately −80° C. to approximately 150° C., for example at from −80 to −60° C., at room temperature, at from −20 to 40° C. or at reflux temperature, under atmospheric pressure or in a closed vessel, where appropriate under pressure, and/or in an inert atmosphere, for example under an argon or nitrogen atmosphere.

At all stages of the reactions, mixtures of isomers that are formed can be separated into the individual isomers, for example diastereoisomers or enantiomers, or into any desired mixtures of isomers, for example racemates or mixtures of diastereoisomers, for example analogously to the methods described under “Additional process steps”.

The solvents from which those solvents that are suitable for any particular reaction may be selected include those mentioned specifically or, for example, water, esters, such as lower alkyl-lower alkanoates, for example ethyl acetate, ethers, such as aliphatic ethers, for example diethyl ether, or cyclic ethers, for example tetrahydrofurane or dioxane, liquid aromatic hydrocarbons, such as benzene or toluene, alcohols, such as methanol, ethanol or 1- or 2-propanol, nitrites, such as acetonitrile, halogenated hydrocarbons, such as methylene chloride or chloroform, acid amides, such as dimethylformamide or dimethyl acetamide, bases, such as heterocyclic nitrogen bases, for example pyridine or N-methylpyrrolidin-2-one, carboxylic acid anhydrides, such as lower alkanoic acid anhydrides, for example acetic anhydride, cyclic, linear or branched hydrocarbons, such as cyclohexane, hexane or isopentane, or mixtures of those solvents, for example aqueous solutions, unless otherwise indicated in the description of the processes. Such solvent mixtures may also be used in working up, for example by chromatography or partitioning.

The compounds, including their salts, may also be obtained in the form of hydrates, or their crystals may, for example, include the solvent used for crystallization. Different crystalline forms may be present.

The invention relates also to those forms of the process in which a compound obtainable as intermediate at any stage of the process is used as starting material and the remaining process steps are carried out, or in which a starting material is formed under the reaction conditions or is used in the form of a derivative, for example in protected form or in the form of a salt, or a compound obtainable by the process according to the invention is produced under the process conditions and processed further in situ. In the process of the present invention those starting materials are preferably used which result in new compounds of formula (I) described at the beginning as being especially valuable. Special preference is given to reaction conditions that are identical or analogous to those mentioned in the Examples.

### Preferred Embodiments According to the Invention

In the following preferred embodiments, general expression can be replaced by the corresponding more specific definitions provided above and below, thus yielding stronger preferred embodiments of the invention.

Preferred is the USE of compounds of the formula I, tautomers thereof or pharmaceutically acceptable salts thereof, where the tyrosine protein kinase dependent disease to be treated is a proliferative disease depending on any one or more of of the following tyrosine protein kinases: especially c-Abl, Bcr-Abl, c-Kit, c-Raf, Flt-1, Flt-3, KDR, Her-1, PDGFR-kinase, c-Src, RET-receptor kinase, FGF-R1, FGF-R2, FGF-R3, FGF-R4, Ephrin receptor kinases (e.g., EphB2 kinase, EphB4 kinase and related Eph kinases), casein kinases (CK-1, CK-2, G-CK), Pak, ALK, ZAP70, Jak1, Jak2, Axl, Cdk1, cdk4, cdk5, Met, FAK, Pyk2, Syk, Insulin receptor kinase, Tie-2 or constitutively activating mutations of kinases (activating kinases) such as of Bcr-Abl, c-Kit, cdk1, c-Raf, Flt-3, FGF-R3, PDGF-receptors, RET, and Met.

More preferably, compounds of formula (I) may be used to treat a proliferative disease depending on the following kinases: c-abl, Flt-3, KDR, c-Src, RET, EphB4, c-kit, cdk1, FGFR-1, c-raf, Her-1, Ins-R and Tek.

The invention relates especially to a compound of the formula (I), 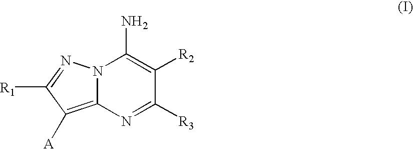
wherein:
R₂ is H; substituted or unsubstituted aryl; substituted or unsubstituted heteroaryl; substituted or unsubstituted aliphatic residue; a functional group; or a substituted or unsubstituted aryl, substituted or unsubstituted heteroaryl or substituted or unsubstituted aliphatic residue which is connected by one connecting group or atom to the pyrazolo[1,5a]pyrimidinyl ring;
R₃ can be H, substituted or unsubstituted aryl, substituted or unsubstituted heteroaryl, substituted or unsubstituted aliphatic residue, a functional group, or an aliphatic residue which may be connected by a connecting group or atom to the pyrazolo[1,5a]pyrimidinyl ring,
at least one of R₂ or R₃ is substituted or unsubstituted aryl; substituted or unsubstituted heteroaryl; or a substituted or unsubstituted heteroaryl or substituted or unsubstituted aryl residue which is connected by one connecting group or atom to the pyrazolo[1,5a]pyrimidinyl ring;
A is H, halogen (such as bromo), an aliphatic moiety, a functional group, substituted or unsubstituted aryl or substituted or unsubstituted heteroaryl; and
R₁ is H, halogen or lower alkyl,
or pharmaceutically acceptable salts thereof,
and use of compounds of formula (I) in the treatment of kinase dependent diseases or for the manufacture of pharmaceutical preparations for the treatment of kinase dependent diseases.

The invention further relates to a compound of the formula (I), 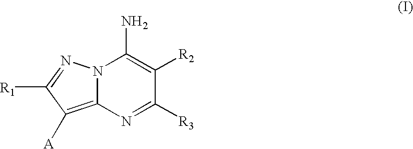
wherein:
R₂ is H; substituted or unsubstituted aryl; substituted or unsubstituted heteroaryl; substituted or unsubstituted aliphatic residue; a functional group; or a substituted or unsubstituted aryl, substituted or unsubstituted heteroaryl or substituted or unsubstituted aliphatic residue which is connected by one connecting group or atom to the pyrazolo[1,5a]pyrimidinyl ring;
R₃ can be H, substituted or unsubstituted aryl, substituted or unsubstituted heteroaryl, substituted or unsubstituted aliphatic residue, a functional group, or a substituted or unsubstituted aliphatic residue which may be connected by a connecting group or atom to the pyrazolo[1,5a]pyrimidinyl ring,
at least one of R₂ or R₃ is substituted or unsubstituted aryl; substituted or unsubstituted heteroaryl; or a substituted or unsubstituted heteroaryl or substituted or unsubstituted aryl residue which is connected by one connecting group or atom to the pyrazolo[1,5a]pyrimidinyl ring;
and provided that R₂ and A cannot both be unsubstituted phenyl;
A is H, halogen (such as bromo), an aliphatic moiety, a functional group, substituted or unsubstituted aryl or heteroaryl; and
R₁ is H, halogen or lower alkyl,
or pharmaceutically acceptable salts thereof,
and use of compounds of formula (I) in the treatment of kinase dependent diseases or for the manufacture of pharmaceutical preparations for the treatment of kinase dependent diseases.

More preferred is a compound of the formula (I), wherein
the connecting atom or group is selected from the group consisting of: alkyl, (such as —CH₂—); oxy —O—; keto —CO—; thio —S—; sulfonyl —SO₂—; sulfoxides —SO—; amines —NH— or —NR—; carboxylic acid; alcohol; esters (—COO—); amides (—CONR—, —CONHR′—); sulfonamides (—SO₂NH—, —SO₂NR′—); (—SO₃—); sulfoxides (—SO—); amino-group; ureas (—NH—CO—NH—, —NR—CO—NH—, —NH—CO—NR—, —NR—CO—NR—); ethers (—O—); carbamates (—NH—CO—O—, —NR—CO—O—); or inverse amides sulfonamides and esters (—NH—CO—, —NR—CO—, —NH—SO₂—, —NR—SO₂—, —OOC—); with alkyl, (such as —CH₂—); oxy —O—; keto —CO—; sulfonyl —SO₂—; sulfonamides (—SO₂NH—, —SO₂NR′—); (—SO₃—); and ureas (—NH—CO—NH—, —NR—CO—NH—, —NH—CO—NR—, —NR—CO—NR—) being especially preferred,
and the functional group is selected from the group consisting of: carboxylic acid; hydroxyl; halogens; cyano (—CN); ethers (—OR); ketones (—CO—R); esters (—COOR); amides (—CONH₂, —CONHR, —CONRR′); thioethers (—SR); sulfonamides (—SO₂NH₂, —SO₂NHR, —SO₂NRR′); sulfones (—SO₂—R); sulfoxides (—SO—R); amines (—NHR, NR′R); ureas (—NH—CO—NH₂, —NH—CO—NHR); ethers (—O—R); halogens; carbamates (—NH—CO—OR); aldehyde-function (—CHO); then also inverse amides; sulfonamides and esters (—NH—CO—R, —NH—SO₂—R, —OOC—R); with halogens; hydroxyl; ethers (—OR); amides (—CONH₂, —CONHR, —CONRR′); sulfonamides (—SO₂NH₂, —SO₂NHR, —SO₂NRR′); amines (—NHR, NR′R); and ureas (—NH—CO—NH₂, —NH—CO—NHR); being especially preferred,
or a pharmaceutically acceptable salt thereof, as such or especially for use in the preparation of a pharmaceutical composition, or for use in the diagnostic or therapeutic treatment of a warm-blooded animal, especially a human.

Especially preferred is a compound of the formula (I), wherein
A is H; a halo (such as Br); or aryl (such as phenyl or benzyl) or heterocyclyl (such as pyridinyl, indolyl or benzothiophenyl),
wherein the aryl or heterocyclyl may be substituted or unsubstituted with up to 4, preferably up to 2 substituents, wherein the substituents are the same or different and are independently selected from halo (such as Cl or Br); hydroxy; amino; amino lower alkyl (such as dimethylamino); amino lower alkoxy (such as ethoxyamine); lower alkyl (such as methyl); lower alkoxy (such as methoxy); substituted or unsubstituted sulfonamide (such as benzo sulfonamide, chlorobenzene sulfonamide or dichloro benzene sulfonamide); carbamates; R₄R₅, wherein R₄ and R₅ can be the same or different and are independently H; lower alkyl (e.g. methyl, ethyl or propyl); or R₄ and R₅ together with the N atom form a 3- to 8-membered heterocyclic ring containing 1-4 nitrogen, oxygen or sulfur atoms (e.g. piperazinyl or lower alkyl piperazinyl) where when R₄ and R₅ together with the N form an heterocyclic ring, said ring may be substituted with 1, 2 or more of any of the substituents described herein, preferably piperazinyl, pyrrolidinyl, alkyl such as methyl, or hydroxy alkyl such as ethanyl. Examples of the heteroring formed by R₄ and R₅ together with the N include morpholinyl, which can be unsubstituted or substituted with methyl or dimethyl; piperazinyl which can be unsubstituted or substituted with 1, 2 or 3 substituents prefereably methyl, oxy or ethanol; or piperadinyl which can be unsubstituted or substituted with 1, 2 or 3 substituents prefereably pyrrolidinyl, amine, alkyl amine, methyl amine, dialkyl amine, dimethylamine or diethylamine;
R₂ is H, C₁-C₃ lower alkyl (such as methyl) or aryl (such as phenyl or benzyl) or heterocyclyl (such as pyridyl, indolyl, thiophenyl, thiazolyl or benzothiophenyl), wherein the aryl or heterocyclyl may be substituted or unsubstituted with up to 4, preferably up to 2 substituents, wherein the substituents are the same or different and are independently selected from halo (such as Cl, F or Br); hydroxy; amino; amino lower alkyl; C₁-C₃ lower alkyl; alkoxy (such as methoxy and benzyloxy where the benzyl ring may be substituted or unsubstituted, such as 3, 4-dichlorobenzyloxy); sulfoamino; substituted or unsubstituted benzosulfonamide (such as 2, 3-dichlorobenzene sulfonamide); substituted or unsubstituted sulfonate (such as chloro-phenyl sulfonate); substituted or unsubstituted ureas (such as 3-trifluoro-methyl-phenyl urea or 4-morpholin-4-yl-3-triflurormethyl-phenyl-urea) or carbamates (such as ethyl-N-phenyl carbamate);
R₃ is H; C₁-C₃ alkyl, methyl; phenyl; pyridinyl or oxaz-5-yl;
or a pharmaceutically acceptable salt thereof, as such or especially for use in the preparation of a pharmaceutical composition, or for use in the diagnostic or therapeutic treatment of a warm-blooded animal, especially a human.

Especially preferred is the use of a compound of formula (I), or a pharmaceutically acceptable salt thereof, in the manufacture of a pharmaceutical preparation for the treatment of a kinase dependent disease.

Also preferred is a compound of the formula (I), or a pharmaceutically acceptable salt thereof, as shown above for use in the treatment of a kinase dependent disease, especially one depending on said kinases and (especially aberrantly highly expressed or constitutively activated) said kinases-dependent disease or disease dependent on the activation of the said kinases pathways or disease dependent on any two or more of said kinases.

In a broader sense of the invention, a kinase dependant disease may be a proliferative disease including a hyperproliferative condition, such as leukemias, hyperplasias, fibrosis (especially pulmonary, but also other types of fibrosis, such as renal fibrosis), angiogenesis, psoriasis, atherosclerosis and smooth muscle proliferation in the blood vessels, such as stenosis or restenosis following angioplasty.

Very preferred is a method of treating a kinase dependent disease comprising administering a compound of formula (I), where the disease to be treated is a proliferative disease, preferably a benign or especially malignant tumor, more preferably carcinoma of the brain, kidney, liver, adrenal gland, bladder, breast, stomach (especially gastric tumors), ovaries, colon, rectum, prostate, pancreas, lung (especially SCLC), vagina, thyroid, sarcoma, glioblastomas, multiple myeloma or gastrointestinal cancer, especially colon carcinoma or colorectal adenoma, or a tumor of the neck and head, an epidermal hyperproliferation, especially psoriasis, prostate hyperplasia, a neoplasia, especially of epithelial character, preferably mammary carcinoma, or a leukemia. Also for the treatment of atherosclerosis, thrombosis, psoriasis, scleroderma and fibrosis.

Compounds of formula (I) are able to bring about the regression of tumors and to prevent the formation of tumor metastases and the growth of (also micro)metastases. In addition they can be used in epidermal hyperproliferation (e.g. psoriasis), in prostate hyperplasia, and in the treatment of neoplasias, especially of epithelial character, for example mammary carcinoma. It is also possible to use the compounds of formula (I) in the treatment of diseases of the immune system insofar as several or, especially, individual tyrosine protein kinases are involved; furthermore, the compounds of formula (I) can be used also in the treatment of diseases of the central or peripheral nervous system where signal transmission by at least one tyrosine protein kinase, especially selected from those mentioned specifically, is involved.

KDR inhibitors are thus especially appropriate for the therapy of diseases related to VEGF receptor tyrosine kinase overexpression. Among these diseases, especially retinopathies, age-related macula degeneration, psoriasis, haemangioblastoma, haemangioma, arteriosclerosis, inflammatory diseases, such as rheumatoid or rheumatic inflammatory diseases, especially arthritis, such as rheumatoid arthritis, or other chronic inflammatory disorders, such as chronic asthma, arterial or post-transplantational atherosclerosis, endometriosis, and especially neoplastic diseases, for example so-called solid tumors (especially cancers of the gastrointestinal tract, the pancreas, breast, stomach, cervix, bladder, kidney, prostate, ovaries, endometrium, lung, brain, melanoma, Kaposi's sarcoma, squamous cell carcinoma of head and neck, malignant pleural mesotherioma, lymphoma or multiple myeloma) and liquid tumors (e.g. leukemias) are especially important.

Flt3 (FMD-like tyrosine kinase) is especially expressed in hematopoietic progenitor cells and in progenitors of the lymphoid and myeloid series. Aberrant expression of the Flt3 gene has been documented in both adult and childhood leukemias including AML (acute myelogenous leukemia), AML with trilineage myelodysplasia (AML/TMDS), ALL (acute lymphoblastic leukemia), CML (chronic myelogenous leukemia) and myelodysplastic syndrome (MDS), which are therefore the preferred diseases to be treated with compounds of the formula I. Activating mutations in Flt3 have been found in approximately 25 to 30% of patients with AML. Thus there is accumulating evidence for the role of Flt3 in human leukemias, and the pyrazolo[1,5a]pyrimidin-7-yl amine derivatives useful according to the invention, especially the compounds of the formula I, as Flt3 inhibitors are especially of use in the therapy of this type of diseases (see Tse et al., Leukemia 15(7), 1001-1010 (2001); Tomoki et al., Cancer Chemother. Pharmacol. 48 (Suppl. 1), S27-S30 (2001); Birkenkamp et al., Leukemia 15(12), 1923-1921 (2001); Kelly et al., Neoplasia 99(1), 310-318 (2002)).

In chronic myelogeous leukemia (CML), a reciprocally balanced chromosomal translocation in hematopoietic stem cells (HSCs) produces the BCR-ABL hybrid gene. The latter encodes the oncogenic Bcr-Abl fusion protein. Whereas ABL encodes a tightly regulated protein tyrosine kinase, which plays a fundamental role in regulating cell proliferation, adherence and apoptosis, the BCR-ABL fusion gene encodes as constitutively activated kinase, which transforms HSCs to produce a phenotype exhibiting deregulated clonal proliferation, reduced capacity to adhere to the bone marrow stroma and a reduces apoptotic response to mutagenic stimuli, which enable it to accumulate progressively more malignant transformations. The resulting granulocytes fail to develop into mature lymphocytes and are released into the circulation, leading to a deficiency in the mature cells and increased susceptibility to infection. ATP-competitive inhibitors of Bcr-Abl have been described which prevent the kinase from activating mitogenic and anti-apoptotic pathways (e.g. P-3 kinase and STAT5), leading to the death of the BCR-ABL phenotype cells and thereby providing an effective therapy against CML. The pyrazolo[1,5a]pyrimidin-7-yl amine derivatives useful according to the present invention, especially the compounds of formula 1, as Bcr-Abl inhibitors are thus especially appropriate for the therapy of diseases related to its overexpression, especially leukemias, such as leukemias, e.g. CML or ALL.

The compounds of formula (I) which inhibit the tyrosine kinase activity of the EGF-R or of the other protein tyrosine kinases mentioned are therefore useful, for example, in the treatment of benign or malignant tumors. The compounds of formula (I) are e.g. able to simultaneously inhibit the growth of tumors with deregulated EGF-R and/or ErbB-2 activity as well as to inhibit the vascularisation of solid tumors triggered by VEGF. This combined activity leads to an improved antitumor effect (see also WO 02/41882). Moreover, the use of a dual inhibitor reduces the risk of drug-drug interactions and further reduces the total drug load as compared to a combination therapy. The compounds of formula (I) are capable of slowing down tumor growth or effecting tumor regression and of preventing the formation of tumor metastases and the growth of micrometastases. They can be used especially in the case of epidermal hyperproliferation (psoriasis), in the treatment of solid cancers like for example non-small cell lung cancer, squameous carcinoma (head and neck), breast, gastric, ovarian, colon and prostate cancers as well as gliomas and in the treatment of leukemias, such as especially acute myeloid leukemia (AML) and chronic myeloid leukemia (CML). In addition, the compounds of formula (I) can be used in the treatment of those disorders of the immune system in which several or, especially, individual protein tyrosine kinases and/or (furthermore) serine/threonine protein kinases are involved; the compounds of formula (I) can also be used in the treatment of those disorders of the central or peripheral nervous system in which signal transmission by several or, especially, a single protein tyrosine kinase(s) and/or (furthermore) serine/threonine protein kinase(s) is/are involved.

Angiogenesis is regarded as an absolute prerequisite for those tumors which grow beyond a maximum diameter of about 1-2 mm; up to this limit, oxygen and nutrients may be supplied to the tumor cells by diffusion. Every tumor, regardless of its origin and its cause, is thus dependent on angiogenesis for its growth after it has reached a certain size.

Three principal mechanisms play an important part in the activity of angiogenesis inhibitors against tumors: 1) Inhibition of the growth of vessels, especially capillaries, into avascular resting tumors, with the result that there is no net tumor growth owing to the balance that is achieved between apoptosis and proliferation; 2) Prevention of the migration of tumor cells owing to the absence of blood flow to and from tumors; and 3) Inhibition of endothelial cell proliferation, thus avoiding the paracrine growth-stimulating effect exerted on the surrounding tissue by the endothelial cells which normally line the vessels.

The present invention can also be used to prevent or treat diseases that are triggered by persistent angiogenesis, such as psoriasis; Kaposi's sarcoma; restenosis, e.g., stent-induced restenosis; endometriosis; Crohn's disease; Hodgkin's disease; leukemia; arthritis, such as rheumatoid arthritis; hemangioma; angiofibroma; eye diseases, such as diabetic retinopathy and neovascular glaucoma; renal diseases, such as glomerulonephritis; diabetic nephropathy; malignant nephrosclerosis; thrombotic microangiopathic syndromes; transplant rejections and glomerulopathy; fibrotic diseases, such as cirrhosis of the liver; mesangial cell-proliferative diseases; arteriosclerosis; injuries of the nerve tissue; and for inhibiting the re-occlusion of vessels after balloon catheter treatment, for use in vascular prosthetics or after inserting mechanical devices for holding vessels open, such as, e.g., stents, as immunosuppressahts, as an aid in scar-free wound healing, and for treating age spots and contact dermatitis.

Most preferred is the use in accordance with the present invention of a compound of the formula (I), or a pharmaceutically acceptable salt thereof, as exemplified hereinbelow under ‘Examples’.

Pharmaceutical Compositions

The invention relates also to pharmaceutical compositions comprising a compound of formula (I), to their use in the therapeutic (in a broader aspect of the invention also prophylactic) treatment or a method of treatment of a kinase dependent disease, especially the preferred diseases mentioned above, to the compounds for said use and to the preparation of pharmaceutical preparations, especially for said uses.

The present invention also relates to pro-drugs of a compound of formula (I) that convert in vivo to the compound of formula (I) as such. Any reference to a compound of formula (I) is therefore to be understood as referring also to the corresponding pro-drugs of the compound of formula (I), as appropriate and expedient.

The pharmacologically acceptable compounds of the present invention may be used, for example, for the preparation of pharmaceutical compositions that comprise an effective amount of a compound of the formula (I), or a pharmaceutically acceptable salt thereof, as active ingredient together or in admixture with a significant amount of one or more inorganic or organic, solid or liquid, pharmaceutically acceptable carriers.

The invention relates also to a pharmaceutical composition that is suitable for administration to a warm-blooded animal, especially a human (or to cells or cell lines derived from a warm-blooded animal, especially a human, e.g. lymphocytes), for the treatment or, in a broader aspect of the invention, prevention of (=prophylaxis against) a disease that responds to inhibition of kinase activity, comprising an amount of a compound of formula (I) or a pharmaceutically acceptable salt thereof, which is effective for said inhibition, especially the in, together with at least one pharmaceutically acceptable carrier.

The pharmaceutical compositions according to the invention are those for enteral, such as nasal, rectal or oral, or parenteral, such as intramuscular or intravenous, administration to warm-blooded animals (especially a human), that comprise an effective dose of the pharmacologically active ingredient, alone or together with a significant amount of a pharmaceutically acceptable carrier. The dose of the active ingredient depends on the species of warm-blooded animal, the body weight, the age and the individual condition, individual pharmacokinetic data, the disease to be treated and the mode of administration.

The invention relates also to a method of treatment for a disease that responds to inhibition of a kinase; which comprises administering an (against the mentioned disease) prophylactically or especially therapeutically effective amount of a compound of formula (I)according to the invention, especially to a warm-blooded animal, for example a human, that, on account of one of the mentioned diseases, requires such treatment.

The dose of a compound of the formula (I) or a pharmaceutically acceptable salt thereof to be administered to warm-blooded animals, for example humans of approximately 70 kg body weight, is preferably from approximately 3 mg to approximately 10 g, more preferably from approximately 10 mg to approximately 1.5 g, most preferably from about 100 mg to about 1000 mg /person/day, divided preferably into 1-3 single doses which may, for example, be of the same size. Usually, children receive half of the adult dose.

The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, dragees, tablets or capsules.

The pharmaceutical compositions of the present invention are prepared in a manner known per se, for example by means of conventional dissolving, lyophilizing, mixing, granulating or confectioning processes.

Solutions of the active ingredient, and also suspensions, and especially isotonic aqueous solutions or suspensions, are preferably used, it being possible, for example in the case of lyophilized compositions that comprise the active ingredient alone or together with a carrier, for example mannitol, for such solutions or suspensions-to be produced prior to use. The pharmaceutical compositions may be sterilized and/or may comprise excipients, for example preservatives, stabilizers, wetting and/or emulsifying agents, solubilizers, salts for regulating the osmotic pressure and/or buffers, and are prepared in a manner known per se, for example by means of conventional dissolving or lyophilizing processes. The said solutions or suspensions may comprise viscosity-increasing substances, such as sodium carboxymethylcellulose, carboxymethylcellulose, dextran, polyvinylpyrrolidone or gelatin.

Suspensions in oil comprise as the oil component the vegetable, synthetic or semi-synthetic oils customary for injection purposes. There may be mentioned as such especially liquid fatty acid esters that contain as the acid component a long-chained fatty acid having from 8-22, especially from 12-22, carbon atoms, for example lauric acid, tridecylic acid, myristic acid, pentadecylic acid, palmitic acid, margaric acid, stearic acid, arachidic acid, behenic acid or corresponding unsaturated acids, for example oleic acid, elaidic acid, erucic acid, brasidic acid or linoleic acid, if desired with the addition of antioxidants, for example vitamin E, β-carotene or 3,5-di-tert-butyl-4-hydroxytoluene. The alcohol component of those fatty acid esters has a maximum of 6 carbon atoms and is a mono- or poly-hydroxy, for example a mono-, di- or tri-hydroxy, alcohol, for example methanol, ethanol, propanol, butanol or pentanol or the isomers thereof, but especially glycol and glycerol. The following examples of fatty acid esters are therefore to be mentioned: ethyl oleate, isopropyl myristate, isopropyl palmitate, “Labrafil M 2375” (polyoxyethylene glycerol trioleate, Gattefosse, Paris), “Miglyol 812” (triglyceride of saturated fatty acids with a chain length of C8 to C12, Huls AG, Germany), but especially vegetable oils, such as cottonseed oil, almond oil, olive oil, castor oil, sesame oil, soybean oil and more especially groundnut oil.

The injection compositions are prepared in customary manner under sterile conditions; the same applies also to introducing the compositions into ampoules or vials and sealing the containers.

Pharmaceutical compositions for oral administration can be obtained by combining the active ingredient with solid carriers, if desired granulating a resulting mixture, and processing the mixture, if desired or necessary, after the addition of appropriate excipients, into tablets, dragee cores or capsules. It is also possible for them to be incorporated into plastics carriers that allow the active ingredients to diffuse or be released in measured amounts.

Suitable carriers are especially fillers, such as sugars, for example lactose, saccharose, mannitol or sorbitol, cellulose preparations and/or calcium phosphates, for example tricalcium phosphate or calcium hydrogen phosphate, and binders, such as starch pastes using for example corn, wheat, rice or potato starch, gelatin, tragacanth, methylcellulose, hydroxypropylmethylcellulose, sodium carboxymethylcellulose and/or polyvinylpyrrolidone, and/or, if desired, disintegrators, such as the above-mentioned starches, and/or carboxymethyl starch, crosslinked polyvinylpyrrolidone, agar, alginic acid or a salt thereof, such as sodium alginate. Excipients are especially flow conditioners and lubricants, for example silicic acid, talc, stearic acid or salts thereof, such as magnesium or calcium stearate, and/or polyethylene glycol. Dragée cores are provided with suitable, optionally enteric, coatings, there being used, inter alia, concentrated sugar solutions which may comprise gum arabic, talc, polyvinylpyrrolidone, polyethylene glycol and/or titanium dioxide, or coating solutions in suitable organic solvents, or, for the preparation of enteric coatings, solutions of suitable cellulose preparations, such as ethylcellulose phthalate or hydroxypropylmethylcellulose phthalate. Capsules are dry-filled capsules made of gelatin and soft sealed capsules made of gelatin and a plasticizer, such as glycerol or sorbitol. The dry-filled capsules may comprise the active ingredient in the form of granules, for example with fillers, such as lactose, binders, such as starches, and/or glidants, such as talc or magnesium stearate, and if desired with stabilizers. In soft capsules the active ingredient is preferably dissolved or suspended in suitable oily excipients, such as fatty oils, paraffin oil or liquid polyethylene glycols, it being possible also for stabilizers and/or antibacterial agents to be added. Dyes or pigments may be added to the tablets or dragee coatings or the capsule casings, for example for identification purposes or to indicate different doses of active ingredient.

Combinations

A compound of the formula (I) may also be used to advantage in combination with other antiproliferative agents. Such antiproliferative agents include, but are not limited to aromatase inhibitors; antiestrogens; topoisomerase I inhibitors; topoisomerase II inhibitors; microtubule active agents; alkylating agents; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; bisphosphonates; biological response modifiers; antiproliferative antibodies; heparanase inhibitors; inhibitors of Ras oncogenic isoforms; telomerase inhibitors; proteasome inhibitors; agents used in the treatment of hematologic malignancies; compounds which target, decrease or inhibit the activity of Flt-3; Hsp90 inhibitors; temozolomide (TEMODAL®); and leucovorin.

The term “aromatase inhibitor” as used herein relates to a compound which inhibits the estrogen production, i.e. the conversion of the substrates androstenedione and testosterone to estrone and estradiol, respectively. The term includes, but is not limited to steroids, especially atamestane, exemestane and formestane and, in particular, non-steroids,. especially aminoglutethimide, roglethimide, pyridoglutethimide, trilostane, testolactone, ketokonazole, vorozole, fadrozole, anastrozole and letrozole. Exemestane can be administered, e.g., in the form as it is marketed, e.g. under the trademark AROMASIN. Formestane can be administered, e.g., in the form as it is marketed, e.g. under the trademark LENTARON. Fadrozole can be administered, e.g., in the form as it is marketed, e.g. under the trademark AFEMA. Anastrozole can be administered, e.g., in. the form as it is marketed, e.g. under the trademark ARIMIDEX. Letrozole can be administered, e.g., in the form as it is marketed, e.g. under the trademark FEMARA or FEMAR. Aminoglutethimide can be administered, e.g., in the form as it is marketed, e.g. under the trademark ORIMETEN. A combination of the invention comprising a chemotherapeutic agent which is an aromatase inhibitor is particularly useful for the treatment of hormone receptor positive tumors, e.g. breast tumors.

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen can be administered, e.g., in the form as it is marketed, e.g. under the trademark NOLVADEX. Raloxifene hydrochloride can be administered, e.g., in the form as it is marketed, e.g. under the trademark EVISTA. Fulvestrant can be formulated as disclosed in U.S. Pat. No. 4,659,516 or it can be administered, e.g., in the form as it is marketed, e.g. under the trademark FASLODEX. A combination of the invention comprising a chemotherapeutic agent which is an antiestrogen is particularly useful for the treatment of estrogen receptor positive tumors, e.g. breast tumors.

The term “anti-androgen” as used herein relates to any substance which is capable of inhibiting the biological effects of androgenic hormones and includes, but is not limited to, bicalutamide (CASODEX), which can be formulated, e.g. as disclosed in U.S. Pat. No. 4,636,505.

The term “gonadorelin agonist” as used herein includes, but is not limited to abarelix, goserelin and goserelin acetate. Goserelin is disclosed in U.S. Pat. No. 4,100,274 and can be administered, e.g., in the form as it is marketed, e.g. under the trademark ZOLADEX. Abarelix can be formulated, e.g. as disclosed in U.S. Pat. No. 5,843,901.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148 (compound A1 in W099/17804). Irinotecan can be administered, e.g. in the form as it is marketed, e.g. under the trademark CAMPTOSAR. Topotecan can be administered, e.g., in the form as it is marketed, e.g. under the trademark HYCAMTIN.

The term “topoisomerase II inhibitor” as used herein includes, but is not limited to the anthracyclines such as doxorubicin (including liposomal formulation, e.g. CAELYX), daunorubicin, epirubicin, idarubicin and nemorubicin, the anthraquinones mitoxantrone and losoxantrone, and the podophillotoxines etoposide and teniposide. Etoposide can be administered, e.g. in the form as it is marketed, e.g. under the trademark ETOPOPHOS. Teniposide can be administered, e.g. in the form as it is marketed, e.g. under the trademark VM 26-BRISTOL. Doxorubicin can be administered, e.g. in the form as it is marketed, e.g. under the trademark ADRIBLASTIN or ADRIAMYCIN. Epirubicin can be administered, e.g. in the form as it is marketed, e.g. under the trademark FARMORUBICIN. Idarubicin can be administered, e.g. in the form as it is marketed, e.g. under the trademark ZAVEDOS. Mitoxantrone can be administered, e.g. in the form as it is marketed, e.g. under the trademark NOVANTRON.

The term “microtubule active agent” relates to microtubule stabilizing, microtubule destabilizing agents and microtublin polymerization inhibitors including, but not limited to taxanes, e.g. paclitaxel and docetaxel, vinca alkaloids, e.g., vinblastine, especially vinblastine sulfate, vincristine especially vincristine sulfate, and vinorelbine, discodermolides, cochicine and epothilones and derivatives thereof, e.g. epothilone B or D or derivatives thereof. Paclitaxel may be administered e.g. in the form as it is marketed, e.g. TAXOL. Docetaxel can be administered, e.g., in the form as it is marketed, e.g. under the trademark TAXOTERE. Vinblastine sulfate can be administered, e.g., in the form as it is marketed, e.g. under the trademark VINBLASTIN R. P. Vincristine sulfate can be administered, e.g., in the form as it is marketed, e.g. under the trademark FARMISTIN. Discodermolide can be obtained, e.g., as disclosed in U.S. Pat. No. 5,010,099. Also included are Epothilone derivatives which are disclosed in WO 98/10121, U.S. Pat. No. 6,194,181, WO 98/25929, WO 98/08849, WO 99/43653, WO 98/22461 and WO 00/31247. Especially preferred are Epothilone A and/or B.

The term “alkylating agent” as used herein includes, but is not limited to, cyclophosphamide, ifosfamide, melphalan or nitrosourea (BCNU or Gliadel). Cyclophosphamide can be administered, e.g., in the form as it is marketed, e.g. under the trademark CYCLOSTIN. Ifosfamide can be administered, e.g., in the form as it is marketed, e.g. under the trademark HOLOXAN.

The term “histone deacetylase inhibitors” or “HDAC inhibitors” relates to compounds which inhibit the histone deacetylase and which possess antiproliferative activity. This includes compounds disclosed in WO 02/22577, especially N-hydroxy-3-[4-[[(2-hydroxyethyl)[2-(1H-indol-3-yl)ethyl]-amino]methyl]phenyl]-2E-2-propenamide, N-hydroxy-3-[4-[[[2-(2-methyl-1H-indol-3-yl)-ethyl]-amino]methyl]phenyl]-2E-2-propenamide and pharmaceutically acceptable salts thereof. It further especially includes Suberoylanilide hydroxamic acid (SAHA).

The term “antineoplastic antimetabolite” includes, but is not limited to, 5-Fluorouracil or 5-FU, capecitabine, gemcitabine, DNA demethylating agents, such as 5-azacytidine and decitabine, methotrexate and edatrexate, and folic acid antagonists such as pemetrexed. Capecitabine can be administered, e.g., in the form as it is marketed, e.g. under the trademark XELODA. Gemcitabine can be administered, e.g., in the form as it is marketed, e.g. under the trademark GEMZAR. Also included is the monoclonal antibody trastuzumab which can be administered, e.g., in the form as it is marketed, e.g. under the trademark HERCEPTIN.

The term “platin compound” as used herein includes, but is not limited to, carboplatin, cis-platin, cisplatinum and oxaliplatin. Carboplatin can be administered, e.g., in the form as it is marketed, e.g. under the trademark CARBOPLAT. Oxaliplatin can be administered, e.g., in the form as it is marketed, e.g. under the trademark ELOXATIN.

The term “compounds targeting/decreasing a protein or lipid kinase activity; or a protein or lipid phosphatase activity; or further anti-angiogenic compounds” as used herein includes, but is not limited to: protein tyrosine kinase and/or serine and/or threonine kinase inhibitors or lipid kinase inhibitors, e.g.:
a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, e.g. a N-phenyl-2-pyrimidine-amine derivative, e.g. imatinib, SU101, SU6668, and GFB-111;
b) compounds targeting, decreasing or inhibiting the activity of the fibroblast growth factor-receptors (FGFR);
c) compounds targeting, decreasing or inhibiting the activity of the insulin-like growth factor receptor I(IGF-IR), such as compounds which target, decrease or inhibit the activity of IGF-IR, especially compounds which inhibit the IGF-IR receptor, such as those compounds disclosed in WO 02/092599;
d) compounds targeting, decreasing or inhibiting the activity of the Trk receptor tyrosine kinase family;
e) compounds targeting, decreasing or inhibiting the activity of the Axl receptor tyrosine kinase family;
f) compounds targeting, decreasing or inhibiting the activity of the c-Met receptor;
g) compounds targeting, decreasing or inhibiting the activity of the Kit/SCFR receptor tyrosine kinase;
h) compounds targeting, decreasing or inhibiting the activity of the C-kit receptor tyrosine kinases—(part of the PDGFR family), such as compounds which target, decrease or inhibit the activity of the c-Kit receptor tyrosine kinase family, especially compounds which inhibit the c-Kit receptor, e.g imatinib;
i) compounds targeting, decreasing or inhibiting the activity of members of the c-Abl family and their gene-fusion products (e.g. BCR-Abl kinase), such as compounds which target decrease or inhibit the activity of c-Abl family members and their gene fusion products, e.g. a N-phenyl-2-pyrimidine-amine derivative, e.g. imatinib; PD180970; AG957; NSC 680410; or PD173955 from ParkeDavis;
j) compounds targeting, decreasing or inhibiting the activity of members of the protein kinase C (PKC) and Raf family of serine/threonine kinases, members of the MEK, SRC, JAK, FAK, PDK and Ras/MAPK family members, or PI(3) kinase family, or of the PI(3)-kinase-related kinase family, and/or members of the cyclin-dependent kinase family (CDK) and are especially those staurosporine derivatives disclosed in U.S. Pat. No. 5,093,330, e.g. midostaurin; examples of further compounds include e.g. UCN-01, safingol, BAY 43-9006, Bryostatin 1, Perifosine; Ilmofosine; RO 318220 and RO 320432; GO 6976; Isis 3521; LY333531/LY379196; isochinoline compounds such as those disclosed in WO 00/09495; FTIs; PD184352 or QAN697( a P13K inhibitor);
k) compounds targeting, decreasing or inhibiting the activity of protein-tyrosine kinase inhibitors, such as compounds which target, decrease or inhibit the activity of protein-tyrosine kinase inhibitors include imatinib mesylate (GLEEVEC) or tyrphostin. A tyrphostin is preferably a low molecular weight (Mr<1500) compound, or a pharmaceutically acceptable salt thereof, especially a compound selected from the benzylidenemalonitrile class or the S-arylbenzenemalonirile or bisubstrate quinoline class of compounds, more especially any compound selected from the group consisting of Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); and
l) compounds targeting, decreasing or inhibiting the activity of the epidermal growth factor family of receptor tyrosine kinases (EGFR, ErbB2, ErbB3, ErbB4 as homo- or heterodimers), such as compounds which target, decrease or inhibit the activity of the epidermal growth factor receptor family are especially compounds, proteins or antibodies which inhibit members of the EGF receptor tyrosine kinase family, e.g. EGF receptor, ErbB2, ErbB3 and ErbB4 or bind to EGF or EGF related ligands, and are in particular those compounds, proteins or monoclonal antibodies generically and specifically disclosed in WO 97/02266, e.g. the compound of ex. 39, or in EP 0 564 409, WO 99/03854, EP 0520722, EP 0 566 226, EP 0 787 722, EP 0 837 063, U.S. Pat. No. 5,747,498, WO 98/10767, WO 97/30034, WO 97/49688, WO 97/38983 and, especially, WO 96/30347 (e.g. compound known as CP 358774), WO 96/33980 (e.g. compound ZD 1839) and WO 95/03283 (e.g. compound ZM105180); e.g. trastuzumab (HERCEPTIN), cetuximab, Iressa, Tarceva, OSI-774, Cl-1033, EKB-569, GW-2016, E1.1, E2.4, E2.5, E6.2, E6.4, E2.11, E6.3 or E7.6.3, and 7H-pyrrolo-[2,3-d]pyrimidine derivatives which are disclosed in WO 03/013541.

Further anti-angiogenic compounds include compounds having another mechanism for their activity, e.g. unrelated to protein or lipid kinase inhibition e.g. thalidomide (THALOMID) and TNP-470.

Compounds which target, decrease or inhibit the activity of a protein or lipid phosphatase are e.g. inhibitors of phosphatase 1, phosphatase 2A, PTEN or CDC25, e.g. okadaic acid or a derivative thereof.

Compounds which induce cell differentiation processes are e.g. retinoic acid, α- γ- or δ-tocopherol or α- γ- or δ-tocotrienol.

The term cyclooxygenase inhibitor as used herein includes, but is not limited to, e.g. Cox-2 inhibitors, 5-alkyl substituted 2-arylaminophenylacetic acid and derivatives, such as celecoxib (CELEBREX), rofecoxib (VIOXX), etoricoxib, valdecoxib or a 5-alkyl-2-arylaminophenylacetic acid, e.g. 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. “Etridonic acid” can be administered, e.g., in the form as it is marketed, e.g. under the trademark DIDRONEL. “Clodronic acid” can be administered, e.g., in the form as it is marketed, e.g. under the trademark BONEFOS. “Tiludronic acid” can be administered, e.g., in the form as it is marketed, e.g. under the trademark SKELID. “Pamidronic acid” can be administered, e.g. in the form as it is marketed, e.g. under the trademark AREDIA™. “Alendronic acid” can be administered, e.g., in the form as it is marketed, e.g. under the trademark FOSAMAX. “Ibandronic acid” can be administered, e.g., in the form as it is marketed, e.g. under the trademark BONDRANAT. “Risedronic acid” can be administered, e.g., in the form as it is marketed, e.g. under the trademark ACTONEL. “Zoledronic acid” can be administered, e.g. in the form as it is marketed, e.g. under the trademark ZOMETA.

The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “heparanase inhibitor” as used herein refers to compounds which target, decrease or inhibit heparin sulphate degradation. The term includes, but is not limited to, PI-88.

The term “biological response modifier” as used herein refers to a lymphokine or interferons, e.g. interferon γ.

The term “inhibitor of Ras oncogenic isoforms”, e.g. H-Ras, K-Ras, or N-Ras, as used herein refers to compounds which target, decrease or inhibit the oncogenic activity of Ras e.g. a “farnesyl transferase inhibitor” e.g. L-744832, DK8G557 or P115777 (Zarnestra).

The term “telomerase inhibitor” as used herein refers to compounds which target, decrease or inhibit the activity of telomerase. Compounds which target, decrease or inhibit the activity of telomerase are especially compounds which inhibit the telomerase receptor, e.g. telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase are e.g. bengamide or a derivative thereof.

The term “proteasome inhibitor” as used herein refers to compounds which target, decrease or inhibit the activity of the proteasome. Compounds which target, decrease or inhibit the activity of the proteasome include e.g. PS-341 and MLN 341.

The term “matrix metalloproteinase inhibitor” or (“MMP inhibitor”) as used herein includes, but is not limited to collagen peptidomimetic and nonpeptidomimetic inhibitors, tetracycline derivatives, e.g. hydroxamate peptidomimetic inhibitor batimastat and its orally bioavailable analogue marimastat (BB-2516), prinomastat (AG3340), metastat (NSC 683551) BMS-279251, BAY 12-9566, TAA21 1, MMI270B or AAJ996.

The term “agents used in the treatment of hematologic malignancies” as used herein includes, but is not limited to FMS-like tyrosine kinase inhibitors e.g. compounds targeting, decreasing or inhibiting the activity of FMS-like tyrosine kinase receptors (Flt-3R); interferon, 1-b-D-arabinofuransylcytosine (ara-c) and bisulfan; and ALK inhibitors e.g. compounds which target, decrease or inhibit anaplastic lymphoma kinase.

Compounds which target, decrease or inhibit the activity of FMS-like tyrosine kinase receptors (Flt-3R) are especially compounds, proteins or antibodies which inhibit members of the Flt-3R receptor kinase family, e.g. PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteasome pathway. Compounds targeting, decreasing or inhibiting the intrinsic ATPase activity of HSP90 are especially compounds, proteins or antibodies which inhibit the ATPase activity of HSP90 e.g,17-allylamino,17-demethoxygeldanamycin (17AAG), a geldanamycin derivative; other geldanamycin related compounds; radicicol and HDAC inhibitors.

The term “antiproliferative antibodies” as used herein includes, but is not limited to trastuzumab (Herceptin™), Trastuzumab-DM1, erlotinib (Tarceva™), bevacizumab (Avastin™), rituximab (Rituxan®), PRO64553 (anti-CD40) and 2C4 Antibody. By antibodies is meant e.g. intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds of formula (I) can be used in combination with standard leukemia therapies, especially in combination with therapies used for the treatment of AML. In particular, compounds of formula (I) can be administered in combination with e.g. farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412.

The term “antileukemic compounds” includes, for example, Ara-C, a pyrimidine analog, which is the 2′-alpha-hydroxy ribose (arabinoside) derivative of deoxycytidine. Also included is the purine analog of hypoxanthine, 6-mercaptopurine (6-MP) and fludarabine phosphate.

Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), Trichostatin A and compounds disclosed in U.S. Pat. No. 6,552,065, in particular, N-hydroxy-3-[4-[[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl)(2-(1H-indol-3-yl)ethyl]-amino]methyl]phenyl]-2E-2-propenamide, or a pharmaceutically acceptable salt thereof, especially the lactate salt.

Compounds which target, decrease or inhibit the activity of serine/theronine mTOR kinase are especially compounds, proteins or antibodies which inhibit members of the mTOR kinase family e.g. RAD, RAD001, CCI-779, ABT578, SAR543, rapamycin and derivatives thereof; AP23573 from Ariad; everolimus (CERTICAN); and sirolimus.

Somatostatin receptor antagonists as used herein refers to agents which target, treat or inhibit the somatostatin receptor such as octreoride, and SOM230.

Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4th Edition, Vol. 1, pp. 248-275 (1993).

The term EDG binders as used herein refers a class of immunosuppressants that modulates lymphocyte recirculation, such as FTY720.

CERTICAN (everolimus, RAD) an investigational novel proliferation signal inhibitor that prevents proliferation of T-cells and vascular smooth muscle cells.

The term ribonucleotide reductase inhibitors refers to pyrimidine or puring nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives, such as PL-1, PL-2, PL-3, PL-4, PL-5, PL-6, PL-7 or PL-8 mentioned in Nandy et al., Acta Oncologica, Vol. 33, No. 8, pp. 953-961 (1994).

The term “S-adenosylmethionine decarboxylase inhibitors” as used herein includes, but is not limited to the compounds disclosed in U.S. Pat. No. 5,461,076.

Also included are in particular those compounds, proteins or monoclonal antibodies of VEGF disclosed in WO 98/35958, e.g. 1-(4-chloroanilino)-4-(4-pyridylmethyl)phthalazine or a pharmaceutically acceptable salt thereof, e.g. the succinate, or in WO 00/09495, WO 00/27820, WO 00/59509, WO 98/11223, WO 00/27819 and EP 0 769 947; those as described by Prewett et al, Cancer Res, Vol. 59, pp. 5209-5218 (1999); Yuan et al., Proc Natl Acad Sci U S A, Vol. 93, pp.14765-14770 (1996); Zhu et al., Cancer Res, Vol. 58, pp. 3209-3214 (1998); and Mordenti et al., Toxicol Pathol, Vol. 27, No. 1, pp.14-21 (1999); in WO 00/37502 and WO 94/10202; ANGIOSTATIN, described by O'Reilly et al., Cell, Vol. 79, pp. 315-328 (1994); ENDOSTATIN, described by O'Reilly et al., Cell, Vol. 88, pp. 277-285 (1997); anthranilic acid amides; ZD4190; ZD6474; SU5416; SU6668; bevacizumab; or anti-VEGF antibodies or anti-VEGF receptor antibodies, e.g. rhuMAb and RHUFab, VEGF aptamer e.g. Macugon; FLT-4 inhibitors, FLT-3 inhibitors, VEGFR-2 IgG1 antibody, Angiozyme (RPI 4610) and Avastan.

Photodynamic therapy as used herein refers to therapy which uses certain chemicals known as photosensitizing agents to treat or prevent cancers. Examples of photodynamic therapy includes treatment with agents, such as e.g. VISUDYNE and porfimer sodium.

Angiostatic steroids as used herein refers to agents which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to agents, such as e.g. fluocinolone, dexamethasone.

Other chemotherapeutic agents include, but are not limited to, plant alkaloids, hormonal agents and antagonists; biological response modifiers, preferably lymphokines or interferons; antisense oligonucleotides or oligonucleotide derivatives; or miscellaneous agents or agents with other or unknown mechanism of action.

The structure of the active agents identified by code nos., generic or trade names may be taken from the actual edition of the standard compendium “The Merck Index” or from databases, e.g. Patents International (e.g. IMS World Publications).

The above-mentioned compounds, which can be used in combination with a compound of the formula (I), can be prepared and administered as described in the art such as in the documents cited above.

A compound of the formula (I) may also be used to advantage in combination with known therapeutic processes, e.g., the administration of hormones or especially radiation.

A compound of formula (I) may in particular be used as a radiosensitizer, especially for the treatment of tumors which exhibit poor sensitivity to radiotherapy.

By “combination”, there is meant either a fixed combination in one dosage unit form, or a kit of parts for the combined administration where a compound of the formula (I) and a combination partner may be administered independently at the same time or separately within time intervals that especially allow that the combination partners show a cooperative, e.g. synergistic, effect, or any combination thereof.

### EXAMPLES

The following examples serve to illustrate the invention without limiting the scope thereof:

### Abbreviations

DMSO dimethylsulfoxide
ES-MS electrospray mass spectrometry
EtOAc Ethyl Acetate
HPLC high-pressure liquid chromatography
mL mililiter(s)
NMR nuclear magnetic resonance
RT room temperature
^{A}t_{RET} HPLC retention time in minutes (method A)
^{B}t_{RET} HPLC retention time in minutes (method B)
^{C}t_{RET} HPLC retention time in minutes (method C)
^{D}t_{RET} HPLC retention time in minutes (method D)
TFA trifluoroacetic acid
THF tetrahydrofuran
TMSCI Trimethylsilyl chloride

Where no temperatures are given, the reaction takes place at ambient (room) temperature.

Ratios of solvents, e.g., in eluents or solvent mixtures, are given in volume by volume (v/v).

Syntheses

Flash chromatography is performed by using silica gel (Merck; 40-63 μm). For thin layer chromatograhy, pre-coated silica gel (Merck 60 F254) plates are used. Detection of the components is made by UV light (254 nm). HPLC is performed on an Agilent HP 1100 using a Nucleosil 100-3 C₁₈ HD 125×4.0 mm column [1 mumin.; 20-100% NeCN/0.1% TFA in 7 minutes) (Method A); SpectraSystem SP8800/UV2000 using a Nucleosil 100-5 C₁₈ AB 250×4.6 mm column (2 mL/min.; 2-100% MeCN/0.1% TFA in 10 minutes) (Method B); using a Chromalith Speed ROD RP18 50-4.6 mm column (Merck) (2 mL/min.; 2-100% MeCN/0.1% TFA in 2 minutes) (Method C); or a C8 2.1-50 mm 3 μm column (Waters) (2 mL/min.; 5-95% MeCN/0.1% TFA in 2 minutes) (Method D). ¹H-NMR measurements are performed on a Varian Gemini 400 or a Bruker DRX 500 spectrometer using tetraethylsilane as internal standard. Chemical shifts are expressed in ppm downfield from tetraethylsilane and coupling constants (J) are expressed in Hertz (Hz). Electrospray mass spectra are obtained with a Fisons Instruments VG Platform II. Melting points are measured with a Büchi 510 melting point apparatus. Commercially-available solvents and chemicals are used for syntheses.

Analytical HPLC Conditions:

System 1

Linear gradient 20-100% CH₃CN (0.1% TFA) and H₂0 (0.1% TFA) in 7min+2min 100% CH₃CN (0.1% TFA); detection at 215 nm, flow rate 1 mL/min at 30° C. Column: Nucleosil 100-3 C18HD (125×4 mm).

System 2

Linear gradient 2-100% CH₃CN (0.1% TFA) and H₂0 (0.1% TFA) in 7min+2min 100% CH₃CN (0.1% TFA); detection at 215 nm, flow rate 1 mL/min at 30° C. Column: Nucleosil 100-3 C18HD (125×4 mm).

### Example 1

### 3-{7-Amino-3-[4-(4-methyl-piperazin-1-yl)-phenyl]-pyrazolo[1,5-a]pyrimidin-6-yl}-phenol

6-(3-Benzyloxy-phenyl)-3-[4-(4-methyl-piperazin-1-yl)-phenyl]-pyrazolo[1,5-a]pyrimidin-7-ylamine (Stage 1.1) (25 mg, 0.051 mmol) dissolved in THF (6 mL) is hydrogenated in the presence of Pd/C (10% Engelhard 4505, 6 mg) for 13 hours. After filtration and evaporating the solvent under reduced pressure, the residue is flash chromatographed (silica gel, CH₂Cl₂/MeOH/NH₃=95:5:0.1) to give compound of Example 1 as white solid (14 mg, 0.035 mmol; 70%): ES-MS: M+H=401.1, R_{f}(CH₂Cl₂/MeOH/NH₃=90:10:0.1)=0.33, HPLC: ^{A}t_{Ret}=2.77 minutes.

¹H-NMR (400 MHz, DMSO-d₆): 9.59 (s, 1H, OH), 8.58/8.18 (s/s, 1H/1H, pyrazolopyrimidinyl), 8.01 (d, 9.0 Hz, 2H, phenyl), 7.48 (s, 2H, NH₂), 7.32 (t, 8.5 Hz, 1H, phenyl-OH), 6.99 (d, 9.0 Hz, 2H, phenyl), 6.96 (d, 8.5 Hz, 1 H, phenyl-OH), 6.93 (s, 1H, phenyl-OH), 6.80 (d, 8.5 Hz, 1H, phenyl-OH), 3.17/2.48 (m/m, 4H/4H, piperazinyl), 2.24 (s, 3H, CH₃).

### Stage 1.1 6-(3-Benzyloxy-phenyl)-3-[4-(4-methyl-piperazin-1-yl)-phenyl)-pyrazolo[1,5-a]pyrimidin-7-ylamine

4-(4-(4-methyl-piperazin-1-yl)-phenyl)-2H-pyrazol-3-ylamine (Stage 1.2) (100 mg, 0.388 mmol), 2-(3-benzyloxy-phenyl)-3-oxo-propionitrile (Stage 1.3) (98 mg, 0.388 mmol), HCl (2.5 mM in EtOH; 1,55 mmol, 0.9 mL) dissolved in EtOH (1 mL) are stirred for 17 hours at RT. After adding H₂O (4 mL) and K₂CO₃ (250 mg), the reaction mixture is extracted with CH₂Cl₂ (20 mL, 2×). The combined organic phases are washed with H₂O (10 mL), dried (Na₂SO₄), concentrated under reduced pressure and flash chromatographed (silica gel, 2.5×15 cm, CH₂Cl₂/MeOH=9:1) to give compound of Stage 1.1 as white solid (60 mg, 0.122 mmol; 32%); ES-MS: M+H=491.0, R_{f}(CH₂Cl₂/MeOH/NH₃=90:10:0.1)=0.42; HPLC: ^{A}t_{Ret}=4.69 minutes.

¹H-NMR (400 MHz, DMSO-d₆): 8.79/8.21 (s/s, 1H/1H, pyrazolopyrimidinyl), 8.03 (d, 9.0 Hz, 2H, phenyl), 7.53 (s, 2H, NH₂), 7.44 (m, 5H, benzyl), 7.32 (t, 8.5 Hz, 1H, phenyl-OH), 7.29 (s, 1H, pheny-OH), 7.13 (d, 8.5 Hz, 1H, phenyl-OH), 7.06 (d, 8.5 Hz, 1H, phenyl-OH), 6.97 (d, 9.0 Hz, 2H, phenyl), 5.19 (s, 2H, benzyl), 3.17/2.48 (m/m, 4H/4H, piperazinyl), 2.24 (s, 3H, CH₃).

### Stage 1.2 4-(4-(4-Methyl-piperazin-1-yl)-phenyl)-2H-pyrazol-3-ylamine

2-[4-(4-Methyl-piperazin-1-yl)-phenyl]-3-oxo-propionitrile (Stage 1.4) (370 mg, 1.52 mmol), hydrazine monohydrate (0.185 mL, 3.8 mmol) dissolved in AcOH are stirred at 98° C. for 3 hours. After cooling down to RT, H₂O (8 mL) and concentrated HCl (0.8 mL) are added and the reaction mixture is stirred under reflux for 20 minutes. After cooling down to RT, the reaction mixture is adjusted to alkaline pH by slowly adding NH₃ (25%). Precipitating material is filtered-off and kept for further purification. The reaction solution is extracted with CH₂Cl₂ (50 mL, 3×), dried (Na₂SO₄) and concentrated under reduced pressure. Precipitated and extracted material is combined and flash chromatographed (silica gel, 3.0×18 cm, CH₂Cl₂/MeOH/NH₃=9:1:01) to give compound of Stage 1.2 as white solid (277 mg, 1.08 mmol; 71%); ES-MS: M+H=258.1, R_{f}(CH₂Cl₂/MeOH/NH₃=90:10:0.1)=0.28; HPLC: ^{A}t_{Ret}=4.33 minutes.

¹H-NMR (400 MHz, DMSO-d₆): 11.55 (s/broad, 1H, NH), 7.55 (s, 1H, pyrolyl), 7.35 (d, 9.0 Hz, 2H, phenyl), 6.91 (d, 9.0 Hz, 2H, phenyl), 4.55 (s/broad, 2H, NH₂), 3.10/2.46 (m/m, 4H/4H, piperazinyl), 2.23 (s, 3H, CH₃).

### Stage 1.3 2-(3-Benzyloxy-phenyl)-3-oxo-propionitrile

Na (260 mg, 11.3 mmol) is dissolved in absolute EtOH (11 mL) under Ar during 20 minutes. After adding (3-benzylox-phenyl)-acetonitrile (1.9 g, 8.68 mmol) and ethyl formate (1.05 mL, 13.0 mmol), the reaction mixture is stirred under reflux for 2 hours. After evaporating the solvent under reduced pressure, adding H₂O (20 mL), and adjusting to pH=4.0 by adding AcOH, the reaction suspension is extracted with CH₂Cl₂ (30 mL, 2×). The combined organic phases are washed with H₂O (10 mL), dried (Na₂SO₄), concentrated under reduced pressure and flash chromatographed (silica gel, 4.5×25 cm, CH₂Cl₂/MeOH=98:2) to give compound of Stage 1.3 as white solid (780 mg, 3.11 mmol; 36%); ES-MS: M−H=250.0, R_{f}(CH₂Cl₂/MeOH=95:5)=0.49; HPLC: ^{A}t_{Ret}=6.07 minutes.

¹H-NMR (400 MHz, DMSO-d₆): 7.45-7.25/6.98-6.88 (m/m, 8 H, aryl), 5.09 (s, 2H, CH₂), 3.98 (s, 2H, CH₂).

### Stage 1.4 2-[4-(4-Methyl-piperazin-1-yl)-phenyl]-3-oxo-propionitrile

Na 160 mg (7.0 mmol) is dissolved in absolute EtOH (6 mL) under Ar during 10 minutes. After adding [4-(4-methyl-piperazin-1-yl)-phenyl]-acetonitrile (Stage 1.5) (1 g, 4.64 mmol) and ethyl formate (0.56 mL, 7.0 mmol), the reaction mixture is stirred under reflux for 1 hour. After washing the reaction pulp with ether (50 mL, 3×), the solid residue is dissolved in H₂O (60 mL) and adjusted to pH=3.9 by adding AcOH. The aqueous solution is extracted with CH₂Cl₂ (50 mL, 3×). The combined organic phases are washed with H₂O (50 mL). Both aqueous phases are combined and lyophilized. The resulting residue is crystallized from MeOH/CH₂Cl₂ to give compound of Stage 1.4 as white crystals (721 mg, 3.0 mmol; 64%); ES-MS: M+H=244.1; HPLC: ^{A}t_{Ret}=2.43 minutes.

¹H-NMR (400 MHz, DMSO-d₆): the compound forms a tautomeric equilibrium in solution: 7.87/7.77 (s/s, 1H, CH═/CH—OH), 7.53/7.17 (d/d, 9.0 Hz, 2H, phenyl), 7.84/7.82 (d/d, 9.0 Hz, 2H, phenyl), 3.10 (m, 4H, piperazinyl), 2.57/2.51 (m/m, 4H, piperazinyl), 2.29/2.26 (s, 3H, CH₃).

### Stage 1.5 [4-(4-Methyl-piperazin-1-yl)-phenyl]-acetonitrile

(4-Bromo-phenyl)-acetonitrile (5 g, 25.5 mmol), 1-methyl-piperazine (3.4 mL, 30.6 mmol), K₂CO₃ (7.68 g, 35.7 mmol), Pd(AcO)₂ (280 mg, 1.275 mmol), 2-(di-tert-butylphosphino)-biphenyl (1.14 g, 3.825 mmol) dissolved in 1,2-dimethoxyethane (70 mL) are stirred under Ar at 85° C. for 20 hours. After adding H₂O (100 mL), the reaction mixture is extracted with CH₂Cl₂ (100 mL, 3×). The combined organic phases are washed with H₂O (100 mL), dried (Na₂SO₄), concentrated under reduced pressure and flash chromatographed (silica gel, 4.5×34 cm, CH₂Cl₂/MeOH=95:5) to give compound of Stage 1.5 as white solid (2.8 g, 13 mmol; 51%); ES-MS: M+H=216.1; R_{f}(CH₂Cl₂/MeOH=9:1)=0.47; HPLC: ^{A}t_{Ret}=2.24 minutes.

¹H-NMR (400 MHz, DMSO-d₆): 7.14/6.91 (d/d, 9.5 Hz, 2H/2H, phenyl), 7.53 (s, 2H, NH₂), 7.44 (m, 5H, benzyl), 7.32 (t, 8.5 Hz, 1H, phenyl-OH), 7.29 (s, 1H, phenyl-OH), 3.84 (s, 2H, benzyl), 3.09/2.42 (t/t, 5.0 Hz, 4H/4H, piperazinyl), 2.18 (s, 3H, CH₃).

### Example 2

### 6-(3-Methoxy-phenyl)-3-[4-(4-methyl-piperazin-1-yl)-phenyl]-pyrazolo[1,5-a]pyrimidin-7-ylamine

6-(3-Methoxy-phenyl)-3-[4-(4-methyl-piperazin-1-yl)-phenyl]-pyrazolo[1,5-a]pyrimidin-7-ylamine is synthesized by condensation of compound of Stage 1.2 and 2-(3-methoxy-phenyl)-3-oxo-propionitrile (Stage 2.1) analogously to the preparation of compound of Example 1. Yield: 48%, solid powder; ES-MS: M+H=415.1; HPLC: ^{A}t_{Ret}=3.45 minutes.

¹H-NMR (400 MHz, DMSO-d₆): 8.59/8.23 (s/s, 1H/1H, pyrazolopyrimidinyl), 8.06 (d, 9.0 Hz, 2H, phenyl), 7.55 (s, 2H, NH₂), 7.43 (t, 8.5 Hz, 1H, phenyl-OMe), 7.10 (d, 8.5 Hz, 1H, phenyl-OMe), 7.08 (s, 1H, phenyl-OMe), 6.80 (d, 8.5 Hz, 1H, phenyl-OMe), 6.98 (d, 9.0 Hz, 2H, phenyl), 3.83 (s, 3H, CH₃—O), 3.16/2.47 (m/m, 4H/4H, piperazinyl), 2.25 (s, 3H, CH₃).

### Stage 2.1 2-(3-Methoxy-phenyl)-3-oxo-propionitrile

2-(3-Methoxy-phenyl)-3-oxo-propionitrile is synthesized analogously to the preparation of compound of Stage 1.3: Yield: 76%; white powder; ES-MS: M−H=174.0; HPLC: ^{A}t_{Ret}=4.75 minutes.

¹H-NMR (400 MHz, DMSO-d₆): the compound forms a tautomeric equilibrium in solution: 8.09/7.67 (s/s, 1H, CH═/CH—OH), 7.38-7.23 (m, 2H, phenyl), 7.01-6.97 (m, 1H, phenyl), 6.88-6.79 (m, 1H, phenyl), 3.74 (s/broad, 3H, CH₃—O).

### Example 3

### 6-(3,5-Dimethoxy-phenyl)-3-[4-(4-methyl-piperazin-1-yl)-phenyl]-pyrazolo[1,5-a]pyrimidin-7-ylamine

6-(3,5-Dimethoxy-phenyl)-3-[4-(4-methyl-piperazin-1-yl)-phenyl]-pyrazolo[1,5-a]pyrimidin-7-ylamine is synthesized by condensation of compound of Stage 1.2 and 2-(3,5-dimethoxy-phenyl)-3-oxo-propionitrile (Stage 3.1) analogously to the preparation of compound of Example 1. Yield: 44%, solid powder; ES-MS: M+H=445.0; HPLC: ^{A}t_{Ret}=3.77 minutes.

¹H-NMR (400 MHz, DMSO-d₆): 8.59/8.23 (s/s, 1H/1H, pyrazolopyrimidinyl), 8.06 (d, 9.0 Hz, 2H, phenyl), 7.55 (s, 2H, NH₂), 7.43 (t, 8.5 Hz, 1H, phenyl-OMe), 7.10 (d, 8.4 Hz, 1H, phenyl-OMe), 7.57 (s, 2H, NH₂), 7.01 (d, 9.0 Hz, 2H, phenyl), 6.89 (s, 2H, phenyl-OMe), 6.54 (s, 1H, phenyl-OMe), 3.83 (s, 6H, CH₃—O), 3.16/2.47 (m/m, 4H/4H, piperazinyl), 2.24 (s, 3H, N—CH₃).

### Stage 3.1 2-(3,5-Dimethoxy-phenyl)-3-oxo-propionitrile

2-(3,5-Dimethoxy-phenyl)-3-oxo-propionitrile is synthesized analogously to the preparation of compound of Stage 1.3. Yield: 48%; white powder; ES-MS: M+H=206.0; HPLC: ^{A}t_{Ret}=4.79 minutes.

¹H-NMR (400 MHz, DMSO-d₆): the compound forms a tautomeric equilibrium in solution: 8.11/7.68 (s/s, 1H, CH═/CH—OH), 6.85/6.54 (s/s, 2H, phenyl), 6.44/6.38 (s/s, 1H, phenyl), 3.74 (s/broad, 6H, CH₃—O).

### Example 4

### 6-(3-Benzyloxy-phenyl)-3-[4-(4-methyl-piperazin-1-yl)-phenyl]-pyrazolo[1,5-a]pyrimidin-7-ylamine

Prepared by the Step Disclosed in Stage 1.1.

### Examples 5-69

The following Examples enlisted oh Table 1 are synthesized analogously to the preparation of Example 1. The syntheses of intermediates for the preparation of compounds of Examples 5-69 which are not commercially available are described in the text below Table 1. In cases where the title compounds carry a free amino group (Examples 52-54), the final products are generated from their corresponding nitro-function carrying precursors by hydrogenation in the presence of Pd/C (10%) in THF/MeOH during several hours.

TABLE 1

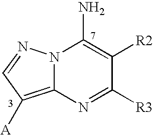

| | | | | Analytical |
|---|---|---|---|---|
| Nb. | A | R2 | R3 | Data |
| 5 | 4-(4-Methyl-piperazin-1- | 4-Chlorophenyl | H | ES-MS [M + 1]⁺ = |
| | yl)phenyl | | | 419.0/421.0; |
| | | | | HPLC ^{A}t_{Ret} = |
| | | | | 3.71 minutes |
| 6 | 4-(4-Methyl-piperazin-1-yl) | 3-Chlorophenyl | H | ES-MS [M + 1]⁺ = |
| | phenyl | | | 419.0/421.0; |
| | | | | HPLC ^{A}t_{Ret} = |
| | | | | 3.92 minutes |
| 7 | 4-(4-Methyl-piperazin-1-yl) | Phenyl | H | ES-MS [M + 1]⁺ = |
| | phenyl | | | 385.1; |
| | | | | PLC ^{A}t_{Ret} = |
| | | | | 3.31 minutes |
| 8 | 4-(4-Methyl-piperazin-1-yl) | Phenyl | Methyl | ES-MS [M + 1]⁺ = |
| | phenyl | | | 399.1; |
| | | | | HPLC ^{A}t_{Ret} = |
| | | | | 3.34 minutes |
| 9 | 4-(4-Methyl-piperazin-1-yl) | Methyl | Phenyl | ES-MS [M + 1]⁺ = |
| | phenyl | | | 399.0; |
| | | | | HPLC ^{A}t_{Ret} = |
| | | | | 3.36 minutes |
| 10 | 4-dimethyl amino phenyl | 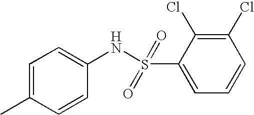 | H | m.p. 143-146° C.; R_{f} (CH₂Cl₂/ MeOH =98:2): 0.32 ES-MS [M + 1]⁺ =552.8; HPLC ^{A}t_{Ret} =4.72 minutes |
| 11 | 4-dimethyl amino phenyl | 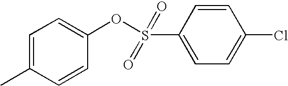 | H | m.p. 186-188° C.; R_{f} (CH₂Cl₂/ MeOH =98:2): 0.50; ES-MS [M + 1]⁺ =519.8; HPLC ^{A}t_{Ret} =5.27 minutes |
| 12 | Phenyl | 4-Methoxyphenyl | Methyl | ES-MS [M + 1]⁺ = |
| | | | | 331 .1; |
| | | | | HPLC ^{B}t_{Ret} = |
| | | | | 6.4 minutes |
| 13 | 4-Methoxy-phenyl | Phenyl | Methyl | ES-MS [M + 1]⁺ = |
| | | | | 331.1; |
| | | | | HPLC ^{B}t_{Ret} = |
| | | | | 6.3 minutes |
| 14 | 4-Methoxy-phenyl | 4-Bromophenyl | Methyl | ES-MS [M + 1]⁺ = |
| | | | | 408.9/410.9; |
| | | | | HPLC ^{B}t_{Ret} = |
| | | | | 7.1 minutes |
| 15 | Phenyl | 4-Bromophenyl | Methyl | ES-MS [M + 1]⁺ = |
| | | | | 378.9/380.9; |
| | | | | HPLC ^{B}t_{Ret} = |
| | | | | 7.0 minutes |
| 16 | Phenyl | 2,6-Dichlorophenyl | H | ES-MS [M + 1]⁺ = |
| | | | | 354.9/356.9; |
| | | | | HPLC ^{B}t_{Ret} = |
| | | | | 7.9 minutes |
| 17 | 3-Methoxy-phenyl | Phenyl | H | ES-MS [M + 1]⁺ = |
| | | | | 317.1; |
| | | | | HPLC ^{B}t_{Ret} = |
| | | | | 6.8 minutes |
| 18 | Br | H | Phenyl | ES-MS [M + 1]⁺ = |
| | | | | 288.9/290.9; |
| | | | | HPLC ^{B}t_{Ret} = |
| | | | | 6.3 minutes |
| 19 | 4-(4-Methyl-piperazin-1-yl) phenyl | 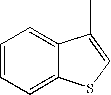 | H | ES-MS [M + 1]⁺ =441.0; HPLC ^{A}t_{Ret} =1.91 minutes |
| 20 | 4-Bromo-phenyl | H | Phenyl | ES-MS [M + 1]⁺ = |
| | | | | 367.0; |
| | | | | HPLC ^{A}t_{Ret} = |
| | | | | 2.56 minutes |
| 21 | 4-(4-Methyl-piperazin-1-yl) phenyl | 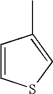 | H | ES-MS [M + 1]⁺ =391.1; HPLC ^{A}t_{Ret} =1.49 minutes |
| 22 | 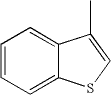 | 3-Methoxyphenyl | H | ES-MS [M + 1]⁺ =373.2; HPLC ^{C}t_{Ret} =2.22 minutes |
| 23 | 4-(4-Methyl-piperazin-1-yl) | Benzyl | H | ES-MS [M + 1]⁺ = |
| | phenyl | | | 399.2; |
| | | | | HPLC ^{C}t_{Ret} = |
| | | | | 1.79 minutes |
| 24 | 3-(4-Methyl-piperazin-1-yl) | 3-Methoxyphenyl | H | ES-MS [M + 1]⁺ = |
| | phenyl | | | 415.2; |
| | | | | HPLC ^{C}t_{Ret} = |
| | | | | 1.82 minutes |
| 25 | 4-(4-Methyl-piperazin-1-yl) phenyl | 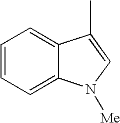 | H | ES-MS [M + 1]⁺=438.2; HPLC ^{C}tᵣₑₜ =1.91 minutes |
| 26 | 4-(4-Methyl-piperazin-1-yl) | 4-Methoxyphenyl | H | ES-MS [M + 1]⁺= |
| | phenyl | | | 415.2; |
| | | | | HPLC ^{C}t_{Ret} = |
| | | | | 2.04 minutes |
| 27 | 4-(4-Methyl-piperazin-1-yl) | 2-Methoxyphenyl | H | ES-MS [M + 1]⁺= |
| | phenyl | | | 415.2; |
| | | | | HPLC ^{C}t_{Ret} = |
| | | | | 1.75 minutes |
| 28 | Pyridin-3-yl | 3-Methoxyphenyl | H | ES-MS [M + 1]⁺= |
| | | | | 318.6; |
| | | | | HPLC ^{C}t_{Ret} = |
| | | | | 1.84 minutes |
| 29 | 3-(4-Methyl-piperazin-1-yl) | 3-Hydroxyphenyl | H | ES-MS [M + 1]⁺= |
| | phenyl | | | 401.6; |
| | | | | HPLC ^{A}t_{Ret} = |
| | | | | 1.78 minutes |
| 30 | 2-Methoxy-5-(4-Methyl- | 3-Benzyloxyphenyl | H | ES-MS [M + 1]⁺= |
| | piperazin-1-yl)phenyl | | | 521.3; |
| | | | | HPLC ^{C}t_{Ret} = |
| | | | | 2.07 minutes |
| 31 | 2-Methoxy-5-(4-Methyl- | 3-Hydroxyphenyl | H | ES-MS [M + 1]⁺= |
| | piperazin-1-yl)phenyl | | | 431.7; |
| | | | | HPLC ^{C}t_{Ret} = |
| | | | | 1.66 minutes |
| 32 | 4-(4-Methyl-piperazin-1-yl) | 2-Benzyloxyphenyl | H | ES-MS [M + 1]⁺= |
| | phenyl | | | 491.2; |
| | | | | HPLC ^{C}t_{Ret} = |
| | | | | 1.77 minutes |
| 33 | 4-(4-Methyl-piperazin-1-yl) | 2-Hydroxyphenyl | H | ES-MS [M + 1]⁺= |
| | phenyl | | | 401.2; |
| | | | | HPLC ^{D}t_{Ret} = |
| | | | | 1.37 minutes |
| 34 | 4-(4-Methyl-piperazin-1-yl) | 4-Benzyloxyphenyl | H | ES-MS [M + 1]⁺= |
| | phenyl | | | 491.2; |
| | | | | HPLC ^{C}t_{Ret} = |
| | | | | 1.85 minutes |
| 35 | 4-(4-Methyl-piperazifl-1-yl) | 4-Hydroxyphenyl | H | ES-MS [M + 1]⁺= |
| | phenyl | | | 401.2; |
| | | | | HPLC ^{D}t_{Ret} = |
| | | | | 1.32 minutes |
| 36 | 3-(4-Methyl-piperazin-1-yl) | 2-Benzyloxyphenyl | H | ES-MS [M + 1]⁺= |
| | phenyl | | | 491.3; |
| | | | | HPLC ^{A}t_{Ret} = |
| | | | | 2.02 minutes |
| 37 | 3-(4-Methyl-piperazin-1-yl) | 2-Hydroxyphenyl | H | ES-MS [M + 1]⁺= |
| | phenyl | | | 401.3; |
| | | | | HPLC ^{A}t_{Ret} = |
| | | | | 1.71 minutes |
| 38 | 3-(4-Methyl-piperazin-1-yl) | 4-Benzyloxyphenyl | H | ES-MS [M + 1]⁺= |
| | phenyl | | | 491.3; |
| | | | | HPLC ^{C}t_{Ret} = |
| | | | | 2.05 minutes |
| 39 | 3-(4-Methyl-piperazin-1-yl) | 4-Hydroxyphenyl | H | ES-MS [M + 1]⁺= |
| | phenyl | | | 401.3; |
| | | | | HPLC ^{C}t_{Ret} = |
| | | | | 1.70 minutes |
| 40 | 2-Methoxy-5-(4-Methyl- | 2-Benzyloxyphenyl | H | ES-MS [M + 1]⁺= |
| | piperazin-1-yl)phenyl | | | 521.3; |
| | | | | HPLC ^{C}t_{Ret} = |
| | | | | 1.99 minutes |
| 41 | 2-Methoxy-5-(4-Methyl- | 2-Hydroxyphenyl | H | ES-MS [M + 1]⁺= |
| | piperazin-1-yl)phenyl | | | 431.3; |
| | | | | HPLC ^{C}t_{Ret} = |
| | | | | 1.70 minutes |
| 42 | 2-Methoxy-5-(4-Methyl- | 4-Benzyloxyphenyl | H | ES-MS [M + 1]⁺= |
| | piperazin-1-yl)phenyl | | | 521.3; |
| | | | | HPLC ^{C}t_{Ret} = |
| | | | | 2.05 minutes |
| 43 | 2-Methoxy-5-(4-Methyl- | 4-Hydroxyphenyl | H | ES-MS [M + 1]⁺= |
| | piperazin-1-yl)phenyl | | | 431.3; |
| | | | | HPLC ^{C}t_{Ret} = |
| | | | | 1.68 minutes |
| 44 | 1-methyl-1H-indol-3-yl | 3-Benzyloxyphenyl | H | ES-MS [M + 1]⁺= |
| | | | | 446.2; |
| | | | | HPLC ^{C}t_{Ret} = |
| | | | | 2.39 minutes |
| 45 | 1-methyl-1H-indol-3-yl | 3-Hydroxyphenyl | H | ES-MS [M + 1]⁺= |
| | | | | 356.6; |
| | | | | HPLC ^{C}t_{Ret} = |
| | | | | 2.06 minutes |
| 46 | 3-Pyridyl | 3-Hydroxyphenyl | H | ES-MS [M + 1]⁺= |
| | | | | 304.1; |
| | | | | HPLC ^{C}t_{Ret} = |
| | | | | 1.72 minutes |
| 47 | 2-methoxy phenyl | 3-Benzyloxyphenyl | H | ES-MS [M + 1]⁺= |
| | | | | 423.2; |
| | | | | HPLC ^{C}t_{Ret} = |
| | | | | 2.10 minutes |
| 48 | 2-methoxy phenyl | 3-Hydroxyphenyl | H | ES-MS [M + 1]⁺= |
| | | | | 333.2; |
| | | | | HPLC ^{C}t_{Ret} = |
| | | | | 1.98 minutes |
| 49 | 3-(4-Methyl-piperazin-1-yl) phenyl | 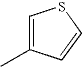 | H | ES-MS [M + 1]⁺=391.1; HPLC ^{D}t_{Ret} =1.56 minutes |
| 50 | 2-Methoxy-5-(4-Methyl- piperazin-1-yl)phenyl | 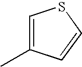 | H | ES-MS [M + 1]⁺=421.1; HPLC ^{D}t_{Ret} =1.49 minutes |
| 51 | 4-(4-Methyl-piperazin-1-yl) phenyl | 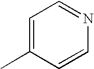 | H | ES-MS [M + 1]⁺=386.2; HPLC ^{C}t_{Ret} =0.44 minutes |
| 52 | 3-(4-Methyl-piperazin-1-yl) phenyl | 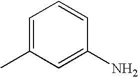 | H | ES-MS [M + 1]⁺=400.2; HPLC ^{C}t_{Ret} =1.57 minutes |
| 53 | 4-(4-Methyl-piperazin-1-yl) phenyl | 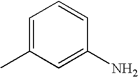 | H | ES-MS [M + 1]⁺=400.0; HPCL ^{D}t_{Ret} =1.75 minutes |
| 54 | 4-(4-Methyl-piperazin-1-yl) phenyl | 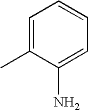 | H | ES-MS [M + 1]⁺=400.2; HPLC ^{D}t_{Ret} =1.40 minutes |
| 55 | 4-(4-Methyl-piperazin-1-yl) phenyl | 4-methyl thiazol-2-yl | H | ES-MS [M + 1]⁺= |
| | phenyl | | | 405.6; |
| | | | | HPLC ^{C}t_{Ret} = |
| | | | | 2.11 minutes |
| 56 | 2-Methoxy-5-(4-Methyl- piperazin-1-yl)phenyl | 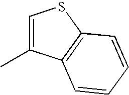 | H | ES-MS [M + 1]⁺=471.5; HPLC ^{C}t_{Ret} =1.80 minutes |
| 57 | 4-methoxy phenyl | 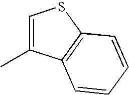 | H | ES-MS [M + 1]⁺=373.7; HPLC ^{A}t_{Ret} =2.24 minutes |
| 58 | 3-methoxy phenyl | 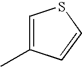 | H | ES-MS [M + 1]⁺=323.1; HPLC ^{C}t_{Ret} =2.09 minutes |
| 59 | 3-methoxy phenyl | 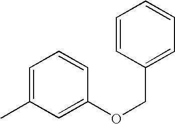 | H | ES-MS [M + 1]⁺=423.2; HPLC ^{C}t_{Ret} =2.38 minutes |
| 60 | 3-methoxy phenyl | 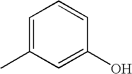 | H | ES-MS [M + 1]⁺=333.6; HPLC ^{C}t_{Ret} =2.02 minutes |
| 61 | 4-(4-Methyl-piperazin-1-yl) phenyl | 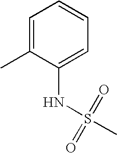 | H | ES-MS [M + 1]⁺=448.2; HPLC: ^{C}t_{Ret} =1.62 minutes |
| 62 | 4-(4-Methyl-piperazin-1-yl) phenyl | 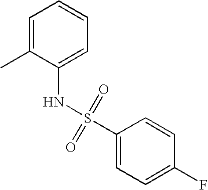 | H | ES-MS [M + 1]⁺=558.2; HPLC: ^{C}t_{Ret} =1.87 minutes |
| 63 | 4-(4-Methyl-piperazin-1-yl) phenyl | 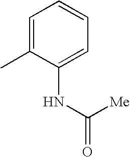 | H | ES-MS [M + 1]⁺=442.1; HPLC: ^{D}t_{Ret} =1.39 minutes |
| 64 | 3-(4-Methyl-piperazin-1-yl) phenyl | 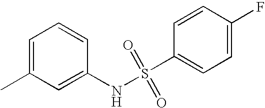 | H | ES-MS [M + 1]⁺=558.4; HPLC: ^{C}t_{Ret} =2.00 minutes |
| 65 | 3-(4-Methyl-piperazin-1-yl) phenyl | 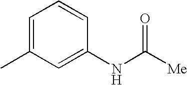 | H | ES-MS [M + 1]⁺=442.6; HPLC: ^{C}t_{Ret} =1.70 minutes |
| 66 | 4-(4-Methyl-piperazin-1-yl) phenyl | 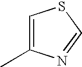 | H | ES-MS [M + 1]⁺=391.5; HPLC: ^{C}t_{Ret} =1.79 minutes |
| 67 | 4-(4-Methyl-piperazin-1-yl) phenyl | 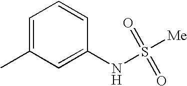 | H | ES-MS [M + 1]⁺=47824; HPLC: ^{C}t_{Ret} =1.76 minutes |
| 68 | 4-(4-Methyl-piperazin-1-yl) phenyl | 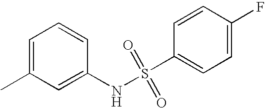 | H | ES-MS [M + 1]⁺=558.2; HPLC: ^{D}t_{Ret} =1.94 minutes |
| 69 | 4-(4-Methyl-piperazin-1-yl) phenyl | 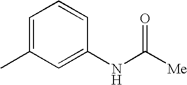 | H | ES-MS [M + 1]⁺=442.2; HPLC: ^{C}t_{Ret} =1.62 minutes |

### Stage 5.1: 2-(4-Chloro-phenyl)-3-oxo-propionitrile

2-(4-Chloro-phenyl)-3-oxo-propionitrile is prepared analogously to the preparation of compound of Stage 1.3: 89%; ES-MS [M−1]⁻=177.9/179.9; HPLC ^{A}t_{Ret}=5.67 minutes.

### Stage 6.1: 2-(3-Chloro-phenyl)-3-oxo-propionitrile

2-(3-Chloro-phenyl)-3-oxo-propionitrile is prepared analogously to the preparation of compound of Stage 1.3: 89%; ES-MS [M−1]⁻=177.9/179.9; HPLC ^{A}t_{Ret}=5.60 minutes.

### Stage 8.1 3-Oxo-2-phenyl-butyronitrile

3-Oxo-2-phenyl-butyronitrile is prepared analogously to the preparation of compound of Stage 1.3: 62%, white crystals, m.p. >215° C.; ES-ES-MS M−H=157.9, R_{f}(hexane/AcOEt=1:1)=0.57.

¹H-NMR (400 MHz, DMSO-d₆): 7.84 (d, 9.0 Hz, 2H), 7.04 (t, 9.0 Hz, 2H), 6.68 (t, 9.0 Hz, 1H), 3.21 (s/broad, 1H, CH), 2.03 (s, 3H, CH₃).

### Stage 9.1 2-Methyl-3-oxo-3-phenyl-propionitrile

2-Methyl-3-oxo-3-phenyl-propionitrile is prepared analogously to the procedure of Yoo et al., Tetrahedron Lett., Vol. 43, No. 27, pp. 4813-4815 (2002). 2-Bromo-propionitrile (0.965 mL, 11.05 mmol) and In-powder (975 mg, 8.5 mmol) are stirred under Ar in THF (15 mL) for 1 hour. After adding benzoylnitrile (735 mg, 5.6 mmol) during 2 minutes, the reaction mixture is stirred at 60° C. in a microwave ofen (Emrys optimizer, personal chemistry, Sweden) for 30 minutes. After filtration over Hyflo and washing with THF (5 mL), the reaction solution is concentrated under reduced pressure and partitioned between ether (150 mL) and phosphate buffer (pH=7, 150 mL). After separation of the organic phase, the aqueous phase is extracted with ether (150 mL). The combined organic phases are washed with brine (30 mL), dried (Na₂SO₄), concentrated under reduced pressure and flash chromatography (silica gel, 2×18 cm, hexane/AcOEt=3:1) to compound of Stage 9.1 as slightly yellowish oil (300 mg, 1.9 mmol; 34%); ES-MS: M−H=157.9; R_{f}(hexane/AcOEt=1:1)=0.60.

¹H-NMR (400 MHz, DMSO-d₆): 8.06 (d, 8.5 Hz, 2H), 7.74 (t, 8.5 Hz, 1H), 7.62 (t, 8.5 Hz, 2H), 5.17 (q, 8.5 Hz, 1H, CH), 1.52 (s, 3H, CH₃).

The compound of Example 10 is synthesized analogously to the preparation of compound of Stage 1.1 by condensing 2,3-dichloro-N-[4-(cyano-formyl-methyl)-phenyl]-benzenesulfonamide (Stage 10.1) and 4-(4-dimethylamino-phenyl)-2H-pyrazol-3-ylamine (Stage 10.3).

### Stage 10.1 2,3-Dichloro-N-[4-(cyano-formyl-methyl)-phenyl]-benzenesulfonamide

Under an atmosphere of N₂ is added portion-wise freshly-cut pieces of sodium (2.3 g total, 100 mmol) to EtOH abs. (230 mL) within 15 minutes which is a slightly exothermic (up to 43° C.). After all sodium is dissolved (ca. 1 hour) 2,3-dichloro-N-(4-cyanomethyl-phenyl)-benzene-sulfonamide (Stage 10.2) (26.27 g, 77 mmol) and formic acid ethyl ester (11.2 mL, 139 mmol) is added to the colorless solution at RT. The mixture is heated to reflux for 2 hours. After cooling to RT, the solvent is removed under reduced pressure and the residue dissolved in H₂O (20 mL), followed by addition of AcOH (200 mL; pH 4). The aqueous layer is extracted with CH₂Cl₂ (2×, 500 mL), the combined organics are washed with H₂O and dried over Na₂SO₄. Purification is done by repeated chromatography (silica gel, EtOAc and CH₂Cl₂/MeOH=98:2) to obtain 2,3-dichloro-N-[4-(cyano-formyl-methyl)-phenyl]-benzenesulfonamide (233 mg, 1% yield) as beige crystals: m.p. 88-102° C.; (CH₂Cl₂/MeOH=95:5): 0.22; ES-MS [M+1]⁺=368; HPLC ^{B}t_{Ret}=5.61 minutes.

### Stage 10.2 2,3-Dichloro-N-(4-cyanomethyl-phenyl)-benzenesulfonamide

To the solution of 4-aminobenzylcyanide (12 g, 90.8 mmol) in pyridine (11 mL) at RT, a solution of 2,3-dichlorobenzene-sulphonylchloride (22.29 g, 90.8 mmol) in THF (80 mL) is added within 20 minutes. The reaction is stirred at reflux for 2 hours. After cooling, the solvent is removed under reduced pressure and the remaining solid suspended in 10% HCl (200 mL). The crude crystalline product is filtered-off, washed with H₂O and dried at 60° C. Final purification is done by suspending the crude compound in MeOH (250 mL), heating to reflux, filtration and drying. 2,3-Dichloro-N-(4-cyanomethyl-phenyl)-benzenesulfonamide (26.54 g, 86%) is obtained as orange crystals: m.p: 202-206° C.; (CH₂Cl₂/MeOH 98:2): 0.54; ES-MS [M−1]⁻=338.8; HPLC ^{B}t_{Ret}=5.85 minutes.

### Stage 10.3 4-(4-Dimethylamino-phenyl)-2H-pyrazol-3-ylamine

4-(4-Dimethylamino-phenyl)-2H-pyrazol-3-ylamine is prepared from 2-(4-dimethylamino-phenyl)-3-oxo-propionitrile (Stage 10.4) and hydrazine hydrate as described in U.S. Pat. No. 2,989,539 (20.6.61; Anderson and Reiff; Example 18). 4-(4-Dimethylamino-phenyl)-2H-pyrazol-3-ylamine: m.p. 173-176° C.; (CH₂Cl₂/MeOH/NH₃=90:10:1): 0.37; ES-MS [M+1]⁺=203; HPLC ^{B}t_{Ret}=1.40 minutes.

### Stage 10.4 2-(4-Dimethylamino-phenyl)-3-oxo-propionitrile

2-(4-Dimethylamino-phenyl)-3-oxo-propionitrile is prepared from (4-dimethylamino-phenyl)-acetonitrile, ethyl formate and sodium as described in U.S. Pat. No.2,989,539 (Example 18).

2-(4-Dimethylamino-phenyl)-3-oxo-propionitrile: m.p. 175-178° C.; ES-MS [M+1]⁺=189; HPLC ^{B}t_{Ret}=2.00 minutes.

The compound of Example 11 is prepared analogously to the synthesis of the compound of Example 10 using 4-(4-dimethylamino-phenyl)-2H-pyrazol-3-ylamine (Stage 10.3) and 4-chloro-benzenesulfonic acid 4-(cyano-formyl-methyl)-phenyl ester (Stage 11.1).

### Stage 11.1 4-Chloro-benzenesulfonic acid 4-(cyano-formyl-methyl)-phenyl ester

4-Chloro-benzenesulfonic acid 4-(cyano-formyl-methyl)-phenyl ester is prepared as described in Example 10 (Stage 10.1), using commercially-available 4-(cyanomethyl)phenyl-4-chlorobenzene-1-sulfonate instead.

4-Chloro-benzenesulfonic acid 4-(cyano-formyl-methyl)-phenyl ester (162 mg); yellowish solid; (CH₂Cl₂/MeOH=95:2): 0.32; ES-MS [M+1]⁺=335; HPLC ^{B}t_{Ret}=6.23 minutes.

### Stage 12.1 2-(4-Methoxy-phenyl)-3-oxo-butyronitrile

2-(4-Methoxy-phenyl)-3-oxo-butyronitrile is prepared as described by Smith, Breen, Hajek and Awang, J. Org. Chem., Vol. 35, No. 7, pp. 2215-2221 (1970).

### Stage 14.1 2-(4-Bromo-phenyl)-3-oxo-butyronitrile

2-(4-Bromo-phenyl)-3-oxo-butyronitrile is synthesized according to the procedure of Rau, Ger. Offen., DE 3001266 (1980).

### Stage 16.1 1,2-(2,6-Dichloro-phenyl)-3-oxo-propionitrile

1,2-(2,6-Dichloro-phenyl)-3-oxo-propionitrile is prepared as described by Menzer, Lankau and Unverferth, Ger. Offen., DE 19521822 (1996).

### Stage 17.1 4-(3-Methoxy-phenyl)-2H-pyrazol-3-ylamine

4-(3-Methoxy-phenyl)-2H-pyrazol-3-ylamine and Stage 22.2 are prepared as described by Bruni et al., Heterocyclic. Chem., Vol. 32, No. 1, pp. 291-298 (1995).

### Stage 19.1 2-Benzo[b]thiophen-3-yl-3-oxo-propionitrile

2-Benzo[b]thiophen-3-yl-3-oxo-propionitrile is synthesized analogously to the preparation of compound of Stage 1.3: Yield: 56%; white powder; ES-MS: M−H=123.9; HPLC: ^{A}t_{Ret}=2.20 minutes.

¹H-NMR (300 MHz, DMSO-d₆): 12.0 (s/broad, 1H), 8.00-7.70 (m, 3H), 7.45-7.35 (m, 2H).

### Stage 21.1 3-Oxo-2-thiophen-3-yl-propionitrile

3-Oxo-2-thiophen-3-yl-propionitrile is synthesized analogously to the preparation of compound of Stage 1.3: Yield: 51%; white powder, ES-MS: M−H=112.9; HPLC: ^{A}t_{Ret}=2.03 minutes.

The compound forms a tautomeric equilibrium in solution: ¹H-NMR (300 MHz, DMSO-d₆): 7.95/7.55 (s/s, 1H, CH═/CH—OH), 7.55-7.50 (m, 2H), 7.30-7.20 (m, 1H).

### Stage 22.1 4-Benzo[b]thiophen-3-yl-1H-pyrazol-3-ylamine

4-Benzo[b]thiophen-3-yl-1H-pyrazol-3-ylamine is synthesized analogously to the preparation of compound of Stage 1.2: Yield: 80%; white powder; ES-MS: M+H=216.0.

¹H-NMR (300 MHz, DMSO-d₆): 12.0 (s/broad, 1H), 8.00-7.80 (m, 2H), 7.75 (s/broad, 1H), 7.60 (s/broad, 1H), 7.40-7.30 (m, 2H).

### Stage 22.2 (2-(3-Methoxy-phenyl)-3-oxo-propionitrile)

(2-(3-Methoxy-phenyl)-3-oxo-propionitrile) is prepared as described by Bruni et al., Heterocyclic. Chem., Vol. 32, No. 1, pp. 291-298 (1995).

### Stage 23.1 2-Formyl-3-phenyl-propionitrile

2-Formyl-3-phenyl-propionitrile is synthesized analogously to the preparation of compound of Stage 1.3: Yield: 77%; oil; ES-MS: M−H=158.0.

The compound forms a tautomeric equilibrium in solution: ¹H-NMR (300 MHz, DMSO-d₆): 7.40-7.15 (m, 5H), 2.85-2.75 (m, 2H).

### Stage 24.1 4 [3-(4-Methyl-piperazin-1-yl)-phenyl]-acetonitrile

4 [3-(4-Methyl-piperazin-1-yl)-phenyl]-acetonitrile is synthesized analogously to the preparation of compound of Stage 1.5: Yield: 55%; brown solid; ES-MS: M+H=216.7; HPLC: ^{C}t_{Ret}=1.65 minutes.

¹H-NMR (300 MHz, CDCl₃): 7.30-7.25 (m, 1H), 6.90-6.82 (m, 2H), 6.80-6.75 (m, 1H), 3.70 (s, 2H), 3.25-3.15 (m, 4H), 2.60-2.50 (m, 4H), 2.35 (s, 3H).

### Stage 24.2 2-[3-(4-Methyl-piperazin-1-yl)-phenyl]-3-oxo-propionitrile

2-[3-(4-Methyl-piperazin-1-yl)-phenyl]-3-oxo-propionitrile is synthesized analogously to the preparation of compound of Stage 1.3: Yield: 100%; brown solid; ES-MS: M+H=244.1; HPLC: ^{C}t_{Ret}=1.67 minutes.

### Stage 24.3 4-[3-(4-Methyl-piperazin-1-yl)-phenyl]-1H-pyrazol-3-ylamine

4-[3-(4-Methyl-piperazin-1-yl)-phenyl]-1H-pyrazol-3-ylamine is synthesized analogously to the preparation of compound of Stage 1.2: Yield: 36%; yellow foam; ES-MS: M+H=258.2; HPLC: ^{C}t_{Ret}=1.46 minutes.

¹H-NMR (300 MHz, CDCl₃): 7.45 (s, 1H), 7.30-7.25 (m, 1H), 7.05-7.00 (m, 1H), 6.95-6.90 (m, 1H), 6.85-6.80 (m, 1H), 4.00 (s/broad, 2H), 3.30-3.20 (m, 4H), 2.65-2.58 (m, 4H), 2.35 (s, 3H).

### Stage 25.1 2-(1-Methyl-1H-indol-3-yl)-3-oxo-propionitrile

2-(1-Methyl-1H-indol-3-yl)-3-oxo-propionitrile is synthesized analogously to the preparation of compound of Stage 1.3: Yield: 59%; oil; ES-MS: M+H=199.1.

The compound forms a tautomeric equilibrium in solution: ¹H-NMR (300 MHz, CDCl₃): 8.00/7.95 (s/s, 1H), 7.60-7.20 (m, 5H), 3.75 (s, 3H).

### Stage 26.1 2-(4-Methoxy-phenyl)-3-oxo-propionitrile

2-(4-Methoxy-phenyl)-3-oxo-propionitrile is synthesized analogously to the preparation of compound of Stage 1.3: Yield: 80%; white solid; ES-MS: M−H=174.3.

The compound forms a tautomeric equilibrium in solution: ¹H-NMR (300 MHz, DMSO-d₆): 7.80/7.58(s/s, 1H), 7.55-7.50 (m, 1H), 7.30-7.20 (m, 1H), 6.90-6.80 (m, 2H), 3.73/3,70 (s/s, 3H).

### Stage 27.1 2-(2-Methoxy-phenyl)-3-oxo-propionitrile

2-(2-Methoxy-phenyl)-3-oxo-propionitrile is synthesized analogously to the preparation of compound of Stage 1.3: Yield: 40%; brown oil; ES-MS: M−H=174.3; HPLC ^{C}t_{Ret}=2.01 minutes.

### Stage 28.1 3-Oxo-2-pyridin-3-yl-propionitrile

3-Oxo-2-pyridin-3-yl-propionitrile is synthesized analogously to the preparation of compound of Stage 1.3: Yield: 71%; brown solid; ES-MS: M+H=147.2; HPLC ^{C}t_{Ret}=1.31 minutes.

### Stage 28.2 4-Pyridin-3-yl-1H-pyrazol-3-ylamine

4-Pyridin-3-yl-1H-pyrazol-3-ylamine is synthesized analogously to the preparation of compound of Stage 1.2: Yield: 68%; brown solid; ES-MS: M+H=161.2; HPLC ^{C}t_{Ret}=0.50 minutes.

### Stage 30.1 [2-Methoxy-5-(4-methyl-piperazin-1-yl)-phenyl]-acetonitrile

[2-Methoxy-5-(4-methyl-piperazin-1-yl)-phenyl]-acetonitrile is synthesized analogously to the preparation of compound of Stage 1.5: Yield: 51%; brown solid; ES-MS: M+H=246.6; HPLC: ^{C}t_{Ret}=1.72 minutes.

¹H-NMR (300 MHz, CDCl₃): 7.00-6.95 (m, 1H), 6.85-6.75 (m, 2H), 3.80 (s, 3H), 3.65 (s, 2H), 3.15-3.05 (m, 4H), 2.60-2.55 (m, 4H), 2.35 (s, 3H).

### Stage 30.2 2-[2-Methoxy-5-(4-methyl-piperazin-1-yl)-phenyl]-3-oxo-propionitrile

2-[2-Methoxy-5-(4-methyl-piperazin-1-yl)-phenyl]-3-oxo-propionitrile is synthesized analogously to the preparation of compound of Stage 1.4: Yield: 100%; brown solid; ES-MS: M+H=274.1; HPLC: ^{C}t_{Ret}=1.62 minutes.

### Stage 30.3 4-[2-Methoxy-5-(4-methyl-piperazin-1-yl)-phenyl]-2H-pyrazol-3-ylamine

4-[2-Methoxy-5-(4-methyl-piperazin-1-yl)-phenyl]-2H-pyrazol-3-ylamine is synthesized analogously to the preparation of compound of Stage 1.2: Yield: 32%; brown solid; ES-MS: M+H=288.2; HPLC: ^{C}t_{Ret}=1.46 minutes.

¹H-NMR (300 MHz, CDCl₃): 7.50 (s, 1H), 7.00-6.95 (m, 1H), 6.90-6.80 (m, 2H), 3.80 (s, 3H), 3.20-3.10 (m, 4H), 2.65-2.55,(m, 4H), 2.35 (s, 3H).

### Stage 32.1 2-(2-Benzyloxy-phenyl)-3-oxo-propionitrile

2-(2-Benzyloxy-phenyl)-3-oxo-propionitrile is synthesized analogously to the preparation of compound of Stage 1.3: Yield: 85%; white solid; ES-MS: M+H=252.6; HPLC: ^{C}t_{Ret}=2.35 minutes.

The compound forms a tautomeric equilibrium in solution: ¹H-NMR (300 MHz, DMSO-d₆): 11.6/7.78 (s, 1H), 7.55-7.45 (m, 2H), 7.40-7.20 (m, 5H), 7.15-7.05 (m, 1H), 7.00-6.90 (m, 1H), 5.15 (s, 2H).

### Stage 34.1 2-(4-Benzyloxy-phenyl)-3-oxo-propionitrile

2-(4-Benzyloxy-phenyl)-3-oxo-propionitrile is synthesized analogously to the preparation of compound of Stage 1.3: Yield: 95%; white solid; ES-MS: M−H=250.3; HPLC: ^{C}t_{Ret}=2.41 minutes.

The compound forms a tautomeric equilibrium in solution: ¹H-NMR (300 MHz, DMSO-d₆): 12.0/11.7 (s, 1H), 7.90-7.80 and 7.60-7.50 (m, 1H), 7.40-7.25 (m, 6H), 7.05-6.95 (m, 2H), 5.10 (s, 2H).

### Stage 44.1 4-(1-Methyl-1H-indol-3-yl)-2H-pyrazol-3-ylamine

4-(1-Methyl-1H-indol-3-yl)-2H-pyrazol-3-ylamine is synthesized analogously to the preparation of compound of Stage 1.2: Yield: 10%; brown foam; ES-MS: M+H=213.2; HPLC: ^{C}t_{Ret}=1.66 minutes.

¹H-NMR (300 MHz, DMSO-d₆): 7.70 (d, 1H), 7.60 (s, 1H), 7.35 (d, 1H), 7.30-7.25 (m, 1H), 7.20-7.10 (m, 2H), 3.80 (s, 3H).

### Stage 47.1 2-(2-Methoxy-phenyl)-3-oxo-propionitrile

2-(2-Methoxy-phenyl)-3-oxo-propionitrile is synthesized analogously to the preparation of compound of Stage 1.3: Yield: 59%; white solid; ES-MS: M+H=175.3; HPLC: ^{C}t_{Ret}=2.01 minutes.

### Stage 47.2 4-(2-Methoxy-phenyl)-2H-pyrazol-3-ylamine

4-(2-Methoxy-phenyl)-2H-pyrazol-3-ylamine is synthesized analogously to the preparation of compound of Stage 1.2: Yield: 35%; white solid; ES-MS: M+H=190.1; HPLC: ^{C}t_{Ret}=1.40 minutes.

¹H-NMR (300 MHz, DMSO-d₆): 11.5 (bs, 1H), 7.50 (bs, 1H), 7.30 (bs, 1H), 7.20-7.05 (m, 1H), 7.00-6.85 (m, 2H), 4.30 (bs, 2H), 3.75 (s, 3H).

### Stage 51.1 3-Oxo-2-pyridin-4-yl-propionitrile

3-Oxo-2-pyridin-4-yl-propionitrile is synthesized analogously to the preparation of compound of Stage 1.3: Yield: 59%; orange solid; ES-MS: M+H=147.2; HPLC: ^{C}t_{Ret}=1.00 minute.

The compound forms a tautomeric equilibrium in solution: ¹H-NMR (300 MHz, DMSO-d₆): 13.1/9.60 (bs, 1H), 9.10 (bs, 1H), 8.20-8.00 (m, 2H), 7.95-7.80 (m, 1H).

### Stage 52.1 (Z)-3-Dimethylamino-2-(3-nitro-phenyl)-acrylonitrile

(3-Nitro-phenyl)-acetonitrile (1.51 g, 9.31 mmol), dimethoxymethyl-dimethyl-amine (6.2 mL, 46.5 mmol) in xylene (30 mL) are stirred at reflux for 1 hour. After adding hexane (20 mL), the reaction mixture is cooled at 0° C. Precipitating material is filtered-off to give compound of Stage 52.1 as brown solid (1.76 g, 8.19 mmol; 88%); ES-MS: M+H=218.1; HPLC: ^{C}t_{Ret}=2.24 minutes.

¹H-NMR (300 MHz, DMSO-d₆): 8.10-8.05 (m, 1H), 7.90-7.85 (m, 1H), 7.75-7.72 (m, 1H), 7.70 (s, 1H), 7.65-7.60 (m, 1H), 3.30 (s, 6H).

### Stage 52.2 3-[3-(4-Methyl-piperazin-1-yl)-phenyl]-6-(3-nitro-phenyl)-pyrazolo[1,5-a]pyrimidin-7-ylamine

4-[3-(4-Methyl-piperazin-1-yl)-phenyl]-1H-pyrazol-3-ylamine (Stage 24.3) (305 mg, 1.18 mmol), (2)-3-dimethylamino-2-(3-nitro-phenyl)-acrylonitrile (Stage 52.1) (335 mg, 1.54 mmol) dissolved in AcOH (10 mL) and BuOH (10 mL) are stirred at reflux for 16 hours. After adding saturated NaHCO₃ aqueous solution, the reaction mixture is extracted with EtOAc (50 mL, 2×). The combined organic phases are washed with H2O (10 mL), dried (Na₂SO₄), concentrated under reduced pressure and flash chromatographed (silica gel, 2.5×15 cm, CH₂Cl₂/MeOH=9:1) to give compound of Stage 52.2 as orange solid (224 mg, 0.52 mmol; 44%); ES-MS: M+H=430.1; HPLC: ^{C}t_{Ret}=1.91 minutes.

¹H-NMR (300 MHz, DMSO-d₆): 8.70 (s, 1H), 8.35-8.30 (m, 1H), 8.25 (s, 1H), 8.22-8.18 (m, 1H), 7.98-7.95 (m, 1H), 7.90 (bs, 2H), 7.80-7.70 (m, 2H), 7.65-7.60 (m, 1H), 7.25-7.18 (m, 1H), 6.80-6.75 (m, 1H), 3.20-3.10 (m, 4H), 2.50-2.40 (m, 4H), 2.20 (s, 3H).

### Stage 53.1 3-[4-(4-Methyl-piperazin-1-yl)-phenyl]-6-(3-nitro-phenyl)-pyrazolo[1,5-a]pyrimidin-7-ylamine

3-[4-(4-Methyl-piperazin-1-yl)-phenyl]-6-(3-nitro-phenyl)-pyrazolo[1,5-a]pyrimidin-7-ylamine is synthesized analogously to the preparation of compound of Stage 52.2: Yield: 30%; red solid; ES-MS: M+H=430.0.

¹H-NMR (300 MHz, DMSO-d₆): 8.60 (s, 1H), 8.35-8.30 (m, 1H), 8.22 (s, 1H), 8.20-8.10 (m, 1H), 8.00 (d, 2H, J=7.9 Hz), 7.95-7.90 (m, 1H), 7.85 (bs, 2H), 7.80-7.75 (m, 1H), 6.95 (d, 1H), J=7.9 Hz), 3.20-3.10 (m, 4H), 2.50-2.40 (m, 4H), 2.20 (s, 3H).

### Stage 54.1 (Z)-3-Dimethylamino-2-(2-nitro-phenyl)-acrylonitrile

(Z)-3-Dimethylamino-2-(2-nitro-phenyl)-acrylonitrile is synthesized analogously to the preparation of compound of Stage 52.1: Yield: 97%; brown solid.

¹H-NMR (300 MHz, DMSO-d₆): 7.82-7.78 (m,1H), 7.62-7.55 (m, 1H), 7.45-7.35 (m, 2H), 7.20 (s, 1H), 3.15 (s, 6H).

### Stage 54.2 3-[4-(4-Methyl-piperazin-1-yl)-phenyl]-6-(2-nitro-phenyl)-pyrazolo[1,5-a]pyrimidin-7-ylamine

3-[4-(4-Methyl-piperazin-1-yl)-phenyl]-6-(2-nitro-phenyl)-pyrazolo[1,5-a]pyrimidin-7-ylamine is synthesized analogously to the preparation of compound of Stage 55.2: brown solidl; ES-MS: M+H=430.0; HPLC: ^{D}t_{Ret}=1.61 minutes.

¹H-NMR (300 MHz, DMSO-d₆): 8.55 (s, 1H), 8.20-8.15 (m, 1H), 8.05-7.95 (m, 3H), 7.82-7.60 (m, 5H), 7.00-6.95 (m, 2H), 3.15-3.05 (m, 4H), 2.45-2.40 (m, 4H), 2.20 (s, 3H).

### Stage 55.1 (E)-3-Dimethylamino-2-(4-methyl-thiazol-2-yl)-acrylonitrile

(E)-3-Dimethylamino-2-(4-methyl-thiazol-2-yl)-acrylonitrile is synthesized analogously to the preparation of compound of Stage 52.1: Yield: 74%; black solid; ES-MS: M+H=194.2; HPLC: ^{C}t_{Ret}=1.57 minutes.

¹H-NMR (300 MHz, DMSO-d₆): 7.76 (s, 1H), 6.60 (s, 1H), 3.25 (bs, 6H), 2.35 (s, 3H).

### Example 61

### 3-[7-Amino-2-methyl-3-[4-(4-methyl-piperazin-1-yl)phenyl]-pyrazolo[1,5-a]pyrimidin-6-yl}-phenol

3-{7-Amino-2-methyl-3-[4-(4-methyl-piperazin-1-yl)phenyl]-pyrazolo[1,5-a]pyrimidin-6-yl)-phenol is synthesized analogously to the preparation of Example 1 by using methyl hydrazine instead of hydrazine when the pyrazole ring is formed: ES-MS: M+H=415.2; HPLC: ^{D}t_{Ret}=1.45 minutes.

¹H-NMR (300 MHz, DMSO-d₆): 9.53 (s, 1H, OH), 2.56 (s, 3H CH₃), 2.24 (s, 3H CH₃).

### Example 62

### (4-{7-Amino-3-[4-(4-methyl-piperazin-1-yl)-phenyl]-pyrazolo[1,5-a]pyrimidin-6-yl}-phenyl)-carbamic acid ethyl ester

4-(4-(4-Methyl-piperazin-1-yl)-phenyl)-2H-pyrazol-3-ylamine (Stage 1.2) (200 mg, 0.81 mmol) and [4-(2-cyano-1-formyl-ethyl)-phenyl]-carbamic acid ethyl ester (Stage 62.1) (275 mg, 0.04 mmol) dissolved in EtOH (4 mL) and ethanolic HCl (1.6 mL, 2.5 N) are stirred under reflux for 17 hours under Ar. After adding H₂O (4 mL) and K₂CO₃ (250 mg), the reaction mixture is extracted with CH₂Cl₂ (20 mL, 2×). The combined organic phases are washed with H₂O (10 mL), dried (Na₂SO₄), concentrated under reduced pressure and flash chromatographed (silica gel, 2.5×15 cm, CH₂Cl₂/MeOH/NH₃=95:5:0.5) to give compound of Example 62 as white solid (58 mg, 0.123 mmol; 15%); ES-MS: M+H=472.0; R_{f}(CH₂Cl₂/MeOH/NH₃=90:10:0.1)=0.42; HPLC: ^{A}t_{Ret}=4.26 minutes.

¹H-NMR (400 MHz, DMSO-d₆): 8.75/8.58 (s/s, 1H/1H, pyrazolopyrimidinyl), 8.03 (d, 9.0 Hz, 2H, phenyl), 7.61 (d, 9 Hz, 2H, phenyl), 7.53 (s, 2H, NH₂), 7.46 (d, 9 Hz, 2H, phenyl), 7.00 (d, 9 Hz, 2H, phenyl), 4.17 (q, 7.5 Hz, 2H, CH₂-Ethyl), 3.17/2.48 (m/m, 4H/4H, piperazinyl), 2.24 (t, 7.5 Hz, 3H, CH₃).

### Stage 62a.1 [4-(Cyano-1-formyl-methyl)-phenyl]-carbamic acid ethyl ester

[4-(Cyano-methyl)-phenyl]-carbamic acid benzyl ester (Stage 62a.2) (1 g, 3.76 mmol) is formylated in analogy to the preparation of Stage 1.3 giving the corresponding carbamic acid ethyl ester (thereby also transforming the benzyl ester function into the ethyl ester function): colorless crystals (654 mg, 2.66 mmol, 70%). ES-MS: M+H=233.0.

¹H-NMR (400 MHz, DMSO-d₆): 4.12 (q/broad, 7.5 Hz, 2H, CH₂-Ethyl), 1.23 (t/broad, 7.5 Hz, 3H, CH₃-Ethyl).

### Stage 62.2 [4-(Cyano-methyl)-phenyl]-carbamic acid benzyl ester

(4-Amino-phenyl)-acetonitrile (2 g, 15.1 mmol) and dibenzyl dicarbonate (4.33 g, 15.1 mmol) dissolved in dioxane (16 mL) are stirred for 1 hour at RT. After evaporating the solvent, the product is isolated by flash chromatography (silica gel, 4.5×25 cm, CH₂Cl₂/MeOH=99:1): white solid (3.82 g, 14.4 mmol; 95%); ES-MS: M−H=265.0; R_{f}(CH₂Cl₂/MeOH=95:5)=0.49; HPLC: ^{A}t_{Ret}=6.32 minutes.

¹H-NMR (400 MHz, DMSO-d₆): 9.82 (s, 1H, NH), 7.51-7.35 (m, 7H, aryl), 7.26 (d, 8.5 Hz, 2H, aryl), 5.15 (s, 2H, CH₂), 3.95 (s, 2H, CH₂).

### (Z-)3-Dimethylamino-2-thiazol-4-yl-acrylonitrile

(Z-)3-Dimethylamino-2-thiazol-4-yl-acrylonitrile is synthesized analogously to the preparation of compound of Stage 52.1: ES-MS [M+1]⁺=180.1; HPLC : ^{C}t_{Ret}=1.91 minutes

Compounds 61, 62, 64, 67, and 68 carrying sulfonamide and acetylamide functions (compounds 63, 65 and 69) are prepared by reacting the amino precursor with the corresponding sulfonic acid chloride or acetic acid anhydride in the presence of pyridine.

### Examples 70 and 71

The compounds in Table 2 and Table 3 are prepared according to Example 1.

TABLE 2
Example 70

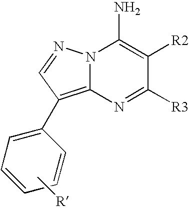

| Nb. | R′ | R2 | R3 |
|---|---|---|---|
| A | 4-(4-Methyl-piperazin-1-yl) | 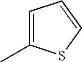 | H |
| B | 4-(-O—(CH₂)₂—NH₂) | 3-Hydroxyphenyl | H |
| C | 4-(-O—(CH₂)₂—NH₂) | H | 4-pyridinyl |
| D | 4-(4-Methyl-piperazin-1-yl) | 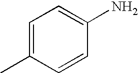 | H |
| E | 4-(4-Methyl-piperazin-1-yl) | 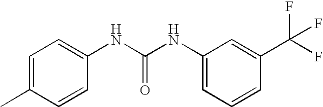 | H |
| F | 4-(4-Methyl-piperazin-1-yl)/2-Cl | 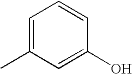 | H |
| G | 4-Dimethylaminyl | 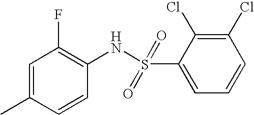 | H |
| H | 4-(4-Methyl-piperazin-1-yl) | 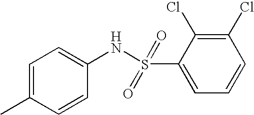 | H |
| I | 4-Dimethylaminyl | 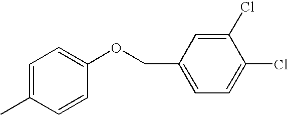 | H |
| K | 4-(4-Methyl-piperazin-1-yl) | 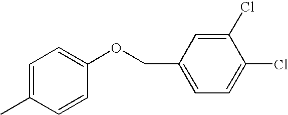 | H |
| L | 4-(4-Methyl-piperazin-1-yl) | 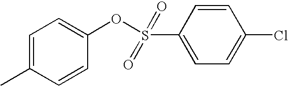 | H |
| M | 4-(4-Methyl-piperazin-1-yl) | H | 4-Pyridinyl |
| N | 4-(4-Methyl-piperazin-1-yl) | 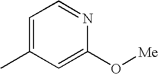 | H |
| O | 4-(4-Methyl-piperazin-1-yl) | 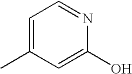 | H |
| R | 4-(4-Methyl-piperazin-1-yl) | 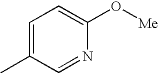 | H |
| S | 4-(4-Methyl-piperazin-1-yl) | 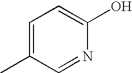 | H |
| V | 4-(4-Methyl-piperazin-1-yl) | H | 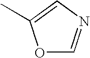 |
| W | 4-(4-Methyl-piperazin-1-yl)/2- Methoxy | 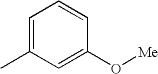 | H |
| X | 4-(4-Methyl-piperazin-1-yl)/2- Methoxy | 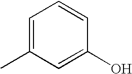 | H |
| Y | 4-(4-Methyl-piperazin-1-yl) | 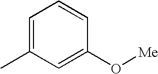 | H |
| Z | 4-(4-Methyl-piperazin-1-yl) | 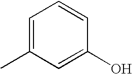 | H |
| z1 | 3-(4-Methyl-piperazin-1-yl) | 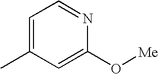 | H |
| z2 | 3-(4-Methyl-piperazin-1-yl) | 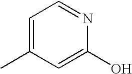 | H |
| z3 | 3-(4-Methyl-piperazin-1-yl) | 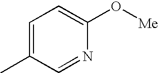 | H |
| z4 | 3-(4-Methyl-piperazin-1-yl) | 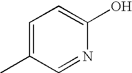 | H |
| z5 | 3-(4-Methyl-piperazin-1-yl) | H | 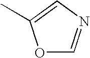 |
| Z6 | 4-(4-Methyl-piperazin-1-yl) | 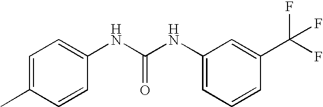 | H |
| Z7 | 3-(4-Methyl-piperazin-1-yl) | 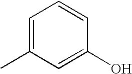 | CH₃ |
| Z8 | 3-(4-Methyl-piperazin-1-yl) | 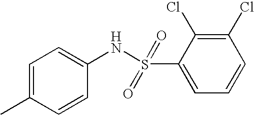 | H |
| Z9 | 4-(4-Methyl-piperazin-1-yl) | 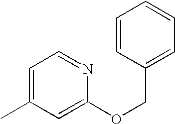 | H |
| z10 | 4-(4-Methyl-piperazin-1-yl) | 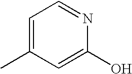 | H |
| z11 | 3-(4-Methyl-piperazin-1-yl) | 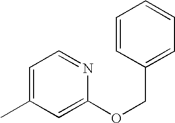 | H |
| z12 | 3-(4-Methyl-piperazin-1-yl) | 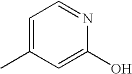 | H |
| z13 | 5-(4-Methyl-piperazin-1-yl)/2- methoxy | 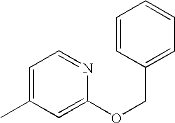 | H |
| z14 | 5-(4-Methyl-piperazin-1-yl)/2- methoxy | 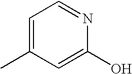 | H |
| z15 | 4-(4-Methyl-piperazin-1-yl)/2- methoxy | 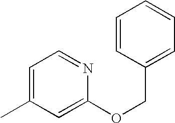 | H |
| z16 | 4-(4-Methyl-piperazin-1-yl)/2- methoxy | 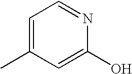 | H |
| z17 | 4-(4-Methyl-piperazin-1-yl) | Br | H |
| z18 | 3-(4-Methyl-piperazin-1-yl) | 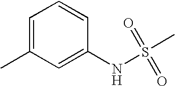 | H |
| Z19 | 4-(4-Methyl-piperazin-1-yl)/2- | 3-Benzyloxyphenyl | H |
| | Methoxy | | |
| z20 | 4-(4-Methyl-piperazin-1-yl)/2- | 3-Hydroxyphenyl | H |
| | Methoxy | | |
| z21 | 3-(4-Methyl-piperazin-1-yl) | 3-Chlorophenyl | Me |
| z22 | 4-(4-Methyl-piperazin-1-yl)/2- | 3-Chlorophenyl | Me |
| | methoxy | | |
| Z23 | 5-(4-Methyl-piperazin-1-yl)/2- | 3-Chlorophenyl | Me |
| | methoxy | | |

TABLE 3
Example 71

| Nb. | A | R2 | R3 |
|---|---|---|---|
| A | Br | H | 4-Pyridinyl |
| B | H | H | 4-Pyridinyl |
| C | Br | 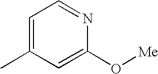 | H |
| D | Br | 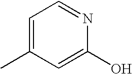 | H |
| E | Br | 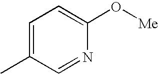 | H |
| F | Br | 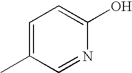 | H |
| G | H | 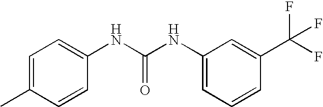 | H |
| H | Pyridin-4-yl | 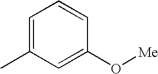 | H |

### Example 72

### 6-(3-Chloro-phenyl)-5-methyl-3-[3-(4-methyl-piperazin-1-yl)-phenyl]-pyrazolo[1,5-a]pyrimidin-7-ylamine

4-[3-(4-Methyl-piperazin-1-yl)-phenyl]-1H-pyrazol-3-ylamine (Stage 72.2) (1.29 g, 5 mmol), is dissolved in EtOH (25 mL), followed by the addition of 2-(3-Chloro-phenyl)-3-oxo-butyronitrile (Stage 72.3) (0.97 g, 5 mmol) and HCl (1.25 M in EtOH; 20 mmol, 16 mL) at RT. The yellowish solution is refluxed under stirring for 20 h. After cooling to RT, H₂O (80 mL) is added as well as K₂CO₃ (2.5 g) to render the mixture basic. The aequeous layer is extracted with CH₂Cl₂ (200 mL, 2×). The combined organic phases are washed with H₂O (50 mL, 2×), dried (Na₂SO₄), concentrated under reduced pressure and chromatographed (silica gel, 120 g RediSep, ISCO Sg-100 CH₂Cl₂/MeOH/NH₃=95:5:0,1) to obtain the title compound 72 as white crystals (1.03 g, 2.38 mmol; 48%); mp. 110-115° C.; MS(ESI+):m/z=433 (M+H)⁺; HPLC: ^{A}t_{Ret}=3.72 minutes (System1).

### Stage 72.1: 2-(3-Chloro-phenyl)-3-oxo-butyronitrile

355 ml of ethanol is heated to 55° C. under N2. To this solution is added sodium (3.91 g; 0.17 mol) within 30 min. and stirred for 1.5 h until all metal is dissolved. 3-Chlorobenzyl cyanide (15.31 g; 0.1 mol) and ethyl acetate (28.53 mL; 0.29 mol) are added to the colorless solution, followed by stirring under reflux for 5 h. After completion of the reaction, the yellow mixture is cooled to rt. and evaporated under reduced pressure. The crude material is taken up into water (200 mL) and neutralized by addition of 25 g of citric acid. The aqueous layer is extracted with CH₂Cl₂ (2×250 mL). The combined organic phases are washed with H₂O (2×150 mL,), dried (Na₂SO₄), concentrated under reduced pressure and chromatographed (silica gel, 1 kg, Merck 60 (0.040-0.063), eluting with EtOAc/Hexanes 1:1) to obtain the title compound 72.1 as yellowish crystals (9.7 g, 0.05 mol; 50%); mp. 92-97° C.; MS(ESI+):m/z=302.9 (M+H)⁺; HPLC: ^{A}t_{Ret}=5.67 minutes (System1).

### Stage 72.2: 4-[3-(4-Methyl-piperazin-1-yl)-phenyl]-1H-pyrazol-3-ylamine

The title compound is prepared as described in example 24; Stage 24.1-24.3

### Example 73

### 6-(3-Chloro-phenyl)-5-methyl-3-[4-(4-methyl-piperazin-1-yl)-phenyl]-pyrazolo[1,5-a]pyrimidin-7-ylamine

The title compound is prepared as described in example 72; using 4-[4-(4-Methyl-piperazin-1-yl)-phenyl]-2H-pyrazol-3-ylamine (Example 1; Stage 1.2) and 2-(3-Chloro-phenyl)-3-oxo-butyronitrile (Example 73, Stage 73.1) instead. Beige crystals; mp. 113-115° C.; MS(ESI+):m/z=433 (M+H)+; HPLC: ^{A}t_{Ret}=3.56 minutes (System1).

### Example 74

### 6-(3-Chloro-phenyl)-3-[2-methoxy-5-(4-methyl-piperazin-1-yl)-phenyl]-5-methyl-pyrazolo[1,5-a]pyrimidin-7-ylamine

The title compound is prepared as described in example 72; using 4-[2-Methoxy-5-(4-methyl-piperazin-1-yl)-phenyl]-2H-pyrazol-3-ylamine and 2-(3-Chloro-phenyl)-3-oxo-butyronitrile (Example 72, Stage 72.1) instead. Beige crystals; mp. 116-121° C.; MS(ESI+):m/z=463 (M+H)⁺; HPLC: ^{A}t_{Ret}=3.68 minutes (System1).

### Stage 74.1: 4-[2-Methoxy-5-(4-methyl-piperazin-1-yl)-phenyl]-2H-pyrazol-3-ylamine

The title compound is prepared as described in example 1, (Stage 1.2; Stage 1.4 and 1.5); using 5-Bromo-2-methoxy-phenylacetonitrile and N-methylpiperazine instead. Yellowish foam; MS(ESI+):m/z=288.2 (M+H)⁺; HPLC: ^{A}t_{Ret}=3.53 minutes (System2).

### Example 75

### 6-(3-Chloro-phenyl)-3-[2-methoxy-4-(4-methyl-piperazin-1-yl)-phenyl]-5-methyl-pyrazolo[1,5-a]pyrimidin-7-ylamine

The title compound is prepared as described in example 72; using 4-[2-Methoxy-4-(4-methyl-piperazin-1-yl)-phenyl]-2H-pyrazol-3-ylamine and 2-(3-Chloro-phenyl)-3-oxo-butyronitrile (Example 72, Stage 72.1) instead. Beige crystals; mp. 215-217° C.; MS(ESI+):m/z=463 (M+H)⁺; HPLC: ^{A}t_{Ret}=3.63 minutes (System1).

Stage 75.1: 4-[2-Methoxy-5-(4-methyl-piperazin-1-yl)-phenyl]-2H-pyrazol-3-ylamine The title compound is prepared as described in example 1, (Stage.1.2; Stage 1.4 and 1.5); using 4-Bromo-2-methoxy-phenylacetonitrile and N-methylpiperazine instead. Green-brown crystals; mp. 173.7-178.1° C.; MS(ESI+):m/z=288.1 (M+H)⁺; HPLC: ^{A}t_{Ret}=3.40, minutes (System2).

### Example 76

### 3-{7-Amino-3-[2-methoxy-4-(4-methyl-piperazin-1-yl)-phenyl]-pyrazolo[1,5-a]pyrimidin-6-yl}-phenol

The title compound is prepared by dissolving 6-(3-Benzyloxy-phenyl)-3-[2-methoxy-4-(4-methyl-piperazin-1-yl)-phenyl]-pyrazolo[1,5-a]pyrimidin-7-ylamine in methanol and subjecting it to catalytic hydrogenation in the presence of Pd/C as described in example 1.: Beige crystals; mp. 217-220° C.; MS(ESI+):m/z=431.0 (M+H)⁺; HPLC: ^{A}t_{Ret}=2.65 minutes (System1).

Stage 76.1: 6-(3-Benzyloxy-phenyl)-3-[2-methoxy-4-(4-methyl-piperazin-1-yl)-phenyl]-pyrazolo[1,5-a]pyrimidin-7-ylamine

The title compound is prepared as described in example 1, (Stage 1.2; Stage 1.4 and 1.5); using 4-Bromo-2-methoxy-phenylacetonitrile and N-methylpiperazine instead. Yellowish solid; MS(ESI⁺):m/z=521 (M+H)⁺; HPLC: ^{A}t_{Ret}=4.38 minutes (System 1).

### Example 77

### 6-(2-Chloro-phenyl)-3-[4-(4-methyl-piperazin-1-yl)-phenyl]-pyrazolo[1,5-a]pyrimidin-7-ylamine

The title compound is prepared as described in example 72; using 4-[4-(4-Methyl-piperazin-1-yl)-phenyl]-2H-pyrazol-3-ylamine and (Z)-2-(2-Chloro-phenyl)-3-dimethylamino-acrylonitrile instead. Yellow solid; mp. 197-200° C.; MS(ESI⁺):m/z=419 (M+H)⁺; HPLC: ^{A}t_{Ret}=3.33 minutes (System1).

### Stage 77.1: (Z)-2-(2-Chloro-phenyl)-3-dimethylamino-acrylonitrile.

N,N-Dimethylformamide-dimethylacetal (9.06 mL; 64.3 mMol) and 2-chlorobenzylcyanide (1.95 g; 12.86 mMol) is heated under stirring to 100° C. under an atmosphere of Argon. After cooling to rt, the mixture is concentrated under reduced pressure and purified by and chromatography (silica gel, 120 g RediSep, ISCO Sg-100, eluting with EtOAc/hexanes 1:1) to obtain the title compound as yellow thick oil (2.44 g, 11.8 mmol; 92%); MS(ESI+):m/z=207 (M+H)⁺; TLC (EtOAc/hexanes 1:1) R_{f}=0.38.

### Example 78

### 6-(2-Chloro-phenyl)-3-[3-(4-methyl-piperazin-1-yl)-phenyl]-pyrazolo[1,5-a]pyrimidin-7-ylamine

The title compound is prepared as described in example 72; using (Z)-2-(2-Chloro-phenyl)-3-dimethylamino-acrylonitrile (Example 77, Stage 77.1) instead. Yellowish crystals; mp. 200-203° C.; MS(ESI+):m/z=419.0 (M+H)⁺; HPLC: ^{A}t_{Ret}=3.65 minutes (System1).

### Example 79

### 6-(4-Fluoro-phenyl)-5-methyl-3-[4-(4-methyl-piperazin-1-yl)-phenyl]-pyrazolo[1,5-a]pyrimidin-7-ylamine

The title compound is prepared as described in example 72; using 4-[4-(4-Methyl-piperazin-1-yl)-phenyl]-2H-pyrazol-3-ylamine and 2-(4-Fluoro-phenyl)-3-oxo-butyronitrile instead. White crystals; mp. 289-291° C.; MS(ESI⁺):m/z=417.1 (M+H)⁺; HPLC: ^{A}t_{Ret}=3.21 minutes (System1).

### Stage 79.1: 2-(4-Fluoro-phenyl)-3-oxo-butyronitrile

The title compound is prepared as described for example 72, Stage 72.1 using (4-Fluoro-phenyl)-acetonitrile instead. Beige crystals; mp. 77-83° C.; MS(ESI⁺):m/z=176.9 (M+H)⁺; HPLC: ^{A}t_{Ret}=5.15 minutes (System1).

### Example 80

### 6-(4-Fluoro-phenyl)-5-methyl-3-[3-(4-methyl-piperazin-1-yl)-phenyl]-pyrazolo[1,5-a]pyrimidin-7-ylamine

The title compound is prepared as described in example 72; using 2-(4-Fluoro-phenyl)-3-oxo-butyronitrile (Example 79, Stage 79.1) instead. White crystals; mp. 204-206° C.; MS(ESI+):m/z=417.1 (M+H)⁺; HPLC: ^{A}t_{Ret}=3.34 minutes (System1).

### Example 81

### 6-(3-Chloro-phenyl)-5-methyl-3-{3-[4-(1-methyl-piperidin-4-yl)-piperazin-1-yl]-phenyl}-pyrazolo[1,5-a]pyrimidin-7-ylamine

The title compound is prepared as described in example 72; using 4-{3-[4-(1-Methyl-piperidin-4-yl)-piperazin-1-yl]-phenyl)-2H-pyrazol-3-ylamine instead. Beige crystals; mp. 180-185° C.; MS(ESI+):m/z=516.0 (M+H)⁺; HPLC: ^{A}t_{Ret}=4.96 minutes (System1).

### Stage 81.1: 4-{3-[4-(1-Methyl-piperidin-4-yl)-piperazin-1-yl]-phenyl}-2H-pyrazol-3-ylamine.

The title compound is prepared as described in example 1, (Stage 1.2 and 1.4 and 1.5); using (3-Bromo-phenyl)-acetonitrile and 1-(1-Methyl-piperidin-4-yl)-piperazine instead. Yellowish crystals; mp. 213-220° C.; MS(ESI⁺): m/z=341.18 (M+H)⁺; HPLC: ^{A}t_{Ret}=3.57 minutes (System1).

### Example 82

### 6-(3-Chloro-4-fluoro-phenyl)-5-methyl-3-[3-(4-methyl-piperazin-1-yl)-phenyl]pyrazolo[1,5-a]pyrimidin-7-ylamine

The title compound is prepared as described in example 72; using 2-(3-Chloro-4-fluoro-phenyl)-3-oxo-butyronitrile instead. White crystals; mp. 224-226° C.; MS(ESI+):m/z=451 (M+H)⁺; HPLC: ^{A}t_{Ret}=3.86 minutes (System1).

### Stage 82.1: 2-(3-Chloro-4-fluoro-phenyl)-3-oxo-butyronitrile

The title compound is prepared as described for example 72, Stage 72.1 using (3-Chloro-4-fluoro-phenyl)-acetonitrile instead. White crystals; mp. 133-134° C.; MS(ESI⁻):m/z=209.9 (M−H)⁻; HPLC: ^{A}t_{Ret}=5.79 minutes (System1).

### Example 83

### 6-(3-Chloro-4-fluoro-phenyl)-5-methyl-3-[4-(4-methyl-piperazin-1-yl)-phenyl]-pyrazolo[1,5-a]pyrimidin-7-ylamine

The title compound is prepared as described for example 72, using 4-[4-(4-Methyl-piperazin-1-yl)-phenyl]-2H-pyrazol-3-ylamine and 2-(3-Chloro-4-fluoro-phenyl)-3-oxo-butyronitrile (Example 82; stage 82.1) instead. White crystals; mp. 264-265° C.; MS(ESI+):m/z=451 (M+H)⁺; HPLC: ^{A}t_{Ret}=3.72 minutes (System1).

### Example 84

### 6-(3-Bromo-phenyl)-5-methyl-3-[3-(4-methyl-piperazin-1-yl)-phenyl]-pyrazolo[1,5-a]pyrimidin-7-ylamine

The title compound is prepared as described for example 72, Stage 72.1 using 2-(3-Bromo-phenyl)-3-oxo-butyronitrile instead. White crystals; mp. 107-113° C.; MS(ESI+):m/z=477 (M+H)⁺; HPLC: ^{A}t_{Ret}=4.90 minutes (System1).

### Stage 84.1: 2-(3-Bromo-phenyl)-3-oxo-butyronitrile

The title compound is prepared as described for example 72, Stage 72.1 using (3-Bromo-phenyl)-acetonitrile instead. White crystals; mp. 96-100° C.; MS(ESI⁻):m/z=235.9 (M−H)⁻; HPLC: ^{A}t_{Ret}=5.76 minutes (System1).

### Example 85

### 6-(3-Bromo-benzyl)-3-[3-(4-methyl-piperazin-1-yl)-phenyl]pyrazolo[1,5-a]pyrimidin-7-ylamine

The title compound is prepared as described in example 72; using 3-(3-Bromo-phenyl)-2-formyl-propionitrile instead. White crystals; mp. 170-171° C.; MS(ESI+):m/z=477.0 (M+H)⁺; HPLC: ^{A}t_{Ret}=3.84 minutes (System1).

### Stage 85.1: 3-(3-Bromo-phenyl)-2-formyl-propionitrile

3-(3-Bromophenyl)propionitrile (0.703 mL; 4.66 mMol) and ethyl formate (1.499 mL; 18.64 mMol) are dissolved in THF anhydrous (12.5 mL) followed by the addition of NaH (60% in mineral oil; 670 mg) at rt. After 17 h at rt, additional NaH (448 mg) and ethyl formate (0.765 mL) is added. Since this results in a strong exothermic reaction, additional solvent is added (15 mL of THF). After completion (3 days), the reaction mixture is cooled to 0° C., treated with a few little ice cubes, followed by addition of 6N HCl (3 mL) to acidify the mixture. After addition of water (50 mL), the mixture is extracted with EtOAc (3×100 mL). The combined organic phases are washed with H₂O (50 mL, 2×), brine, dried (Na₂SO₄), concentrated under reduced pressure and chromatographed (silica gel, 40 g RediSep, ISCO Sg-100, eluting with EtOAc/hexanes 1:1) to obtain the title compound as a brownish oil (220 mg; 20%); MS(ESI−):m/z=235.9 (M−H)−; TLC EtOAc/hexanes 1:1) Rf=0.28.

### Example 86

### 6-(3-Bromo-phenyl)-3-[3-(4-methyl-piperazin-1-yl)-phenyl]-pyrazolo[1,5-a]pyrimidin-7-ylamine

The title compound is prepared as described in example 72; using (Z)-2-(3-Bromo-phenyl)-3-dimethylamino-acrylonitrile instead. White crystals; mp. 195.3-197.2° C.; MS(ESI⁺):m/z=463.0 (M+H)⁺; HPLC: ^{A}t_{Ret}=4.05 minutes (System1).

Stage 86.1: (Z)-2-(3-Bromo-phenyl)-3-dimethylamino-acrylonitrile is prepared as described in example 77, Stage 77.1.: Gold brown crystals; mp. 102-105° C.; MS(ESI⁺):m/z=251.0 (M+H)⁺; HPLC: ^{A}t_{Ret}=6.45 minutes (System1).

### Example 87

### 6-(3-Chloro-phenyl)-5-methyl-3-(3-morpholin-4-yl-phenyl)-pyrazolo[1,5-a]pyrimidin-7-ylamine

The title compound is prepared as described in example 72; using 4-(3-Morpholin-4-yl-phenyl)-2H-pyrazol-3-ylamine instead. Off-white crystals; mp. 165-167° C.; MS(ESI⁺):m/z=420 (M+H)⁺; HPLC: ^{A}t_{Ret}=4.49 minutes (System1).

### Stage 87.1: 4-(3-Morpholin-4-yl-phenyl)-2H-pyrazol-3-ylamine

The title compound is prepared as described in example 1, (Stage 1.2; Stage 1.4 and 1.5); using (3-Bromo-phenyl)-acetonitrile and morpholine instead. Off-white crystals; mp. 166-168° C.; MS(ESI⁺):m/z=245.1 (M+H)⁺; HPLC: ^{A}t_{Ret}=1.79 minutes (System1).

### Example 88

### 6-(3-Chloro-phenyl)-3-(4-methoxy-phenyl)-5-methyl-pyrazolo[1,5-a]pyrimidin-7-ylamine

The title compound is prepared as described in example 72; using 4-(4-Methoxy-phenyl)-2H-pyrazol-3-ylamine instead. White crystals; mp. 171-172° C.; MS(ESI⁺):m/z=365 (M+H)⁺; HPLC: ^{A}t_{Ret}=4.96 minutes (System1).

### Stage 88.1: 4-(4-Methoxy-phenyl)-2H-pyrazol-3-ylamine

The title compound is prepared as described in example 1, (Stage 1.4 and 1.2); using (4-methoxy-phenyl)-acetonitrile instead. White crystals; mp. 198-201° C.; MS(ESI⁺):m/z=190 (M+H)⁺; HPLC: ^{A}t_{Ret}=2.85 minutes (System1).

### Example 89

### 6-(3-Chloro-phenyl)-3-[3-((2R,6S)-2,6-dimethyl-morpholin-4-yl)-phenyl]-5-methyl-pyrazolo[1,5-a]pyrimidin-7-ylamine

The title compound is prepared as described in example 72; using 4-[3-((2R,6S)-2,6-Dimethyl-morpholin-4-yl)-phenyl]-2H-pyrazol-3-ylamine instead. White crystals; mp. 165-167° C.; MS(ESI⁺):m/z=448 (M+H)⁺; HPLC: ^{A}t_{Ret}=5.14 minutes (SYSTEM1).

### Stage 89.1: 4-[3-((2R,6S)-2,6-Dimethyl-morpholin-4-yl)-phenyl]-2H-pyrazol-3-ylamine

The title compound is prepared as described in example 1, (Stage 1.2 and 1.4 and 1.5); using (3-Bromo-phenyl)-acetonitrile and (2R,6S)-2,6-Dimethyl-morpholine instead. White crystals; mp. 158-160° C.; MS(ESI⁺):m/z=273.1 (M+H)⁺; HPLC: ^{A}t_{Ret}=3.02 minutes (System1).

### Example 90

### 2-(4-{3-[7-Amino-6-(3-chloro-phenyl)-5-methyl-pyrazolo[1,5-a]pyrimidin-3-yl]-phenyl}-piperazin-1-yl)-ethanol

The title compound is prepared as described in example 72; using 2-{4-[3-(5-Amino-1H-pyrazol-4-yl)-phenyl]-piperazin-1-yl}-ethanol instead. Off-white crystals; mp. 108-116° C.; MS(ESI⁺):m/z=463 (M+H)⁺; HPLC: ^{A}t_{Ret}=3.62 minutes (System1).

### Stage 90.1: 2-{4-[3-(5-Amino-1H-pyrazol-4-yl)-phenyl]-piperazin-1-yl)-ethanol

The title compound is prepared as described in example 1, (Stage 1.2 and 1.4 and 1.5); using (3-Bromo-phenyl)-acetonitrile and 2-Piperazin-1-yl-ethanol instead. Yellowish foam; mp. 40-48° C.; MS(ESI⁺):m/z=288.1 (M+H)⁺; HPLC: ^{A}t_{Ret}=3.45 minutes (System1).

### Example 91

### 6-Benzyl-3-[3-(4-methyl-piperazin-1-yl)-phenyl]-pyrazolo[1,5-a]pyrimidin-7-ylamine

The title compound is prepared as described in example 72; using 2-Formyl-3-phenyl-propionitrile (Example 23; Stage 23.1) instead. Yellowish crystals; mp. 72-75° C.; MS(ESI+):m/z=399.1 (M+H)⁺; HPLC: ^{A}t_{Ret}=3.30 minutes (System1).

### Example 92

### 6-(3-Chloro-phenyl)-3-(3,4-dimethoxy-phenyl)-5-fluoromethyl-pyrazolo[1,5-a]pyrimidin-7-ylamine

The title compound is prepared as described in example 72; using 4-(3,4-Dimethoxy-phenyl)-2H-pyrazol-3-ylamine (Example 93; Stage 93.1) and 2-(3-Chloro-phenyl)-4-fluoro-3-oxo-butyronitrile instead. Yellow crystals; mp. 228-230° C.; MS(ESI+):m/z=413 (M+H)⁺; HPLC: ^{A}t_{Ret}=6.65 minutes (System1).

### Stage 92.1: 2-(3-Chloro-phenyl)-4-fluoro-3-oxo-butyronitrile

The title compound is prepared as described for example 72, Stage 72.1 using fluoro-acetic acid ethyl ester instead. Beige crystals; mp. 90-96° C.; MS(ESI⁻):m/z=209.9 (M−H)⁻; HPLC: ^{A}t_{Ret}=5.66 minutes (System1).

### Example 93

### 6-(3-Chloro-phenyl)-3-(3,4-dimethoxy-phenyl)-5-methyl-pyrazolo[1,5-a]pyrimidin-7-ylamine

The title compound is prepared as described in example 72; using 4-(3,4-Dimethoxy-phenyl)-2H-pyrazol-3-ylamine instead. Off-white solid; mp. 223-226° C.; MS(ESI⁺):m/z=395.0 (M+H)⁺; HPLC: ^{A}t_{Ret}=4.69 minutes (System1).

### Stage 93.1: 4-(3,4-Dimethoxy-phenyl)-2H-pyrazol-3-ylamine

The title compound is prepared as described in example 1, (Stage 1.4 and 1.2); using (3,4-Dimethoxy-phenyl)-acetonitrile instead. White crystals; mp. 143-146° C.; MS(ESI⁺):m/z=220.1 (M+H)⁺; HPLC: ^{A}t_{Ret}=2.28 minutes (System1).

### Example 94

### 6-(3-Chloro-4-fluoro-phenyl)-3-(3,4-dimethoxy-phenyl)-5-methyl-pyrazolo[1,5-a]pyrimidin-7-ylamine

The title compound is prepared as described in example 72; using 4-(3,4-Dimethoxy-phenyl)-2H-pyrazol-3-ylamine (Example 93; Stage 93.1) and 2-(3-Chloro-4-fluoro-phenyl)-3-oxo-butyronitrile (Example 82; stage 82.1) instead. Off-white solid; mp. 235-238° C.; MS(ESI⁺):m/z=413.0 (M+H)⁺; HPLC: ^{A}t_{Ret}=4.83 minutes (System1).

### Example 95

### 6-(3-Chloro-4-fluoro-phenyl)-3-(4-methoxy-phenyl)-5-methyl-pyrazolo[1,5-a]pyrimidin-7-ylamine

The title compound is prepared as described in example 72; using 4-(4-Methoxy-phenyl)-2H-pyrazol-3-ylamine (Example 88; Stage 88.1) and 2-(3-Chloro-4-fluoro-phenyl)-3-oxo-butyronitrile (Example 82; stage 82.1) instead. White crystals; mp. 224-227° C.; MS(ESI⁺):m/z=383 (M+H)⁺; HPLC: ^{A}t_{Ret}=5.08 minutes (System1).

### Example 96

### 6-(4-Fluoro-phenyl)-3-(4-methoxy-phenyl)-5-methyl-pyrazolo[1,5-a]pyrimidin-7-ylamine

The title compound is prepared as described in example 72; using 4-(4-Methoxy-phenyl)-2H-pyrazol-3-ylamine (Example 88, Stage 88.1) and 2-(4-Fluoro-phenyl)-3-oxo-butyronitrile (Example 79; Stage 79.1) instead. White crystals; mp. 243-244° C.; MS(ESI⁺):m/z=349,1 (M+H)⁺; HPLC: ^{A}t_{Ret}=4.56 minutes (System1).

### Example 97

### 2-(4-{3-[7-Amino-6-(4-fluoro-phenyl)-5-methyl-pyrazolo[1,5-a]pyrimidin-3-yl]-phenyl}-piperazin-1-yl)-ethanol

The title compound is prepared as described in example 72; using 2-{4-[3-(5-Amino-1H-pyrazol-4-yl)-phenyl]-piperazin-1-yl}-ethanol (Example 90, Stage 90.1) and 2-(4-Fluoro-phenyl)-3-oxo-butyronitrile (Example 79; Stage 79.1) instead. Off-white crystals; mp. 209-212° C.; MS(ESI⁺):m/z=447.1 (M+H)⁺; HPLC: ^{A}t_{Ret}=3.24 minutes (System1).

### Example 98

### 6-(3,4-Difluoro-phenyl)-5-methyl-3-[3-(4-methyl-piperazin-1-yl)-phenyl]-pyrazolo[1,5-a]pyrimidin-7-ylamine

The title compound is prepared as described in example 72; using 2-(3,4-difluoro-phenyl)-3-oxo-butyronitrile instead. White solid; mp. 216-219° C.; MS(ESI⁺):m/z=435 (M+H)⁺; HPLC: ^{A}t_{Ret}=3.30 minutes (SYSTEM1).

### Stage 98.1: 2-(3,4-Difluoro-phenyl)-3-oxo-butyronitrile

The title compound is prepared as described in example 1, (Stage 1.4 and, 1.2); using (3,4-difluoro-phenyl)-acetonitrile instead. White crystals;. mp. 147-152° C.; MS(ESI⁺):m/z=195 (M+H)⁺; HPLC: ^{A}t_{Ret}=5.39 minutes (System1).

### Example 99

### 6-(3,4-Difluoro-phenyl)-3-(3,4-dimethoxy-phenyl)-5-methyl-pyrazolo[1,5-a]pyrimidin-7-ylamine

The title compound is prepared as described in example 72; using 4-(3,4-Dimethoxy-phenyl)-2H-pyrazol-3-ylamine (Example 93; Stage 93.1) and 2-(3,4-difluoro-phenyl)-3-oxo-butyronitrile (Example 98; Stage 98.1) instead. Off-white solid; mp. 230-235° C.; MS(ESI⁺):m/z=397.0 (M+H)⁺; HPLC: ^{A}t_{Ret}=4.53 minutes (System1).

### Example 100

### 2-(4-(3-[7-Amino-6-(3-chloro-4-fluoro-phenyl)-5-methyl-pyrazolo[1,5-a]pyrimidin-3-yl]-phenyl}-piperazin-1-yl)-ethanol

The title compound is prepared as described in example 72; using 2-{4-[3-(5-Amino-1H-pyrazol-4-yl)-phenyl]-piperazin-1-yl}-ethanol (Example 90, Stage 90.1) and 2-(3-Chloro-4-fluoro-phenyl)-3-oxo-butyronitrile (Example 82; stage 82.1) instead. Off-white crystals; mp. 104-107° C.; MS(ESI⁺):m/z=481 (M+H)⁺; HPLC: ^{A}t_{Ret}=4.00 minutes (System1).

### Example 101

### 2-(4-{3-[7-Amino-6-(3,4-difluoro-phenyl)-5-methyl-pyrazolo[1,5-a]pyrimidin-3-yl]-phenyl}-piperazin-1-yl)-ethanol

The title compound is prepared as described in example 72; using 2-{4-[3-(5-Amino-1H-pyrazol-4-yl)-phenyl]-piperazin-1-yl)-ethanol (Example 90, Stage 90.1) and 2-(3,4-difluoro-phenyl)-3-oxo-butyronitrile (Example 98; Stage 98.1) instead. Off-white crystals; mp. 172-174° C.; MS(ESI⁺):m/z=465 (M+H)⁺; HPLC: ^{A}t_{Ret}=3.71 minutes (System1).

### Example 102

### 6-(3-Chloro-phenyl)-5-methyl-3-[3-(4-pyrrolidin-1-yl-piperidin-1-yl)-phenyl]-pyrazolo[1,5-a]pyrimidin-7-ylamine

The title compound is prepared as described in example 72; using 4-[3-(4-Pyrrolidin-1-yl-piperidin-1-yl)-phenyl]-1H-pyrazol-3-ylamine instead. Yellow crystals; mp. 188-193° C.; MS(ESI⁺):m/z=487.0 (M+H)⁺; HPLC: ^{A}t_{Ret}=4.21 minutes (System1).

### Stage 102.1: 4-[3-(4-Pyrrolidin-1-yl-piperidin-1-yl)-phenyl]-1H-pyrazol-3-ylamine

The title compound is prepared as described in example 1, (Stage 1.2 and 1.4 and 1.5); using (3-Bromo-phenyl)-acetonitrile and 4-Pyrrolidin-1-yl-piperidine instead. Yellow crystals; mp. 214-216° C.; MS(ESI⁺):m/z=312.1 (M+H)⁺; HPLC: ^{A}t_{Ret}=3.71 minutes (System1).

### Example 103

### 6-(4-Fluoro-phenyl)-5-methyl-3-[3-(4-pyrrolidin-1-yl-piperidin-1-yl)-phenyl]-pyrazolo[1,5-a]pyrimidin-7-ylamine

The title compound is prepared as described in example 72; using 4-[3-(4-Pyrrolidin-1-yl-piperidin-1-yl)-phenyl]-1H-pyrazol-3-ylamine (Example 102; Stage 102.1) and 2-(4-Fluoro-phenyl)-3-oxo-butyronitrile (Example 79; Stage 79.1) instead. White crystals; mp. 244-249° C.; MS(ESI⁺):m/z=471.0 (M+H)⁺; HPLC: ^{A}t_{Ret}=3.82 minutes (System1).

### Example 104

### 6-(3-Chloro-phenyl)-3-[3-(4-diethylamino-piperidin-1-yl)-phenyl]-5-methyl-pyrazolo[1,5-a]pyrimidin-7-ylamine

The title compound is prepared as described in example 72; using {1-[3-(3-Amino-1H-pyrazol-4-yl)-phenyl]-piperidin-4-yl}-diethyl-amine instead. White crystals; mp. 163-168° C.; MS(ESI⁺):m/z=489.0 (M+H)⁺; HPLC: ^{A}t_{Ret}=4.02 minutes (System1).

### Stage 104.1: {1-[3-(3-Amino-1H-pyrazol-4-yl)-phenyl]-piperidin-4-yl}-diethyl-amine

The title compound is prepared as described in example 1, (Stage 1.2 and 1.4 and 1.5); using (3-bromo-phenyl)-acetonitrile and diethyl-piperidin-4-yl-amine instead. Beige solid, amorphous; MS(ESI⁺):m/z=314.2 (M+H)⁺; HPLC: ^{A}t_{Ret}=3.75 minutes (System1).

### Example 105

### 3-[3-(4-Diethylamino-piperidin-1-yl)-phenyl]-6-(4-fluoro-phenyl)-5-methyl-pyrazolo[1,5-a]pyrimidin-7-ylamine

The title compound is prepared as described in example 72; using (1-[3-(3-Amino-1H-pyrazol-4-yl)-phenyl]-piperidin-4-yl}-diethyl-amine (Example 104, Stage 104.1) and 2-(4-Fluoro-phenyl)-3-oxo-butyronitrile (Example 79; Stage 79.1) instead. White crystals; mp. 208-210° C.; MS(ESI⁺):m/z=473.1 (M+H)⁺; HPLC: ^{A}t_{Ret}=3.63 minutes (System1).

### Example 106

### 6-(4-Fluoro-phenyl)-5-methyl-3-[3-(4-methyl-4-oxy-piperazin-1-yl)-phenyl]-pyrazolo[1,5-a]pyrimidin-7-ylamine

6-(4-Fluoro-phenyl)-5-methyl-3-[3-(4-methyl-piperazin-1-yl)-phenyl]-pyrazolo[1,5-a]pyrimidin-7-ylamine (Example 80) (50 mg; 0.12 mMol) is dissolved in CH₂Cl₂ (10 mL) and at 0° C. treated with 3-chloroperbenzoic acid (31.1 mg; 0.126 mMol) for 1 h, followed by stirring at rt for 2 h. After removald of the solvent under reduced pressure, the crude mixture is purified by chromatography (silica gel, 12 g RediSep, ISCO Sg-100 CH₂Cl₂/MeOH/NH₃=80:20:1) to obtain the title compound as beige crystals (44 mg); mp. 210-223° C.; MS(ESI⁺):m/z=449 (M+H)⁺; HPLC: ^{A}t_{Ret}=3.31 minutes (System1).

### Example 107

### 6-(4-Fluoro-phenyl)-5-methyl-3-[3-(4-methyl-1,4-dioxy-piperazin-1-yl)-phenyl]-pyrazolo[1,5-a]pyrimidin-7-ylamine

The title compound is isolated from the same reaction described in Example 106: beige crystals (20 mg); mp. 161-169° C.; MS(ESI+):m/z=433 (M+H)⁺; HPLC:: ^{A}t_{Ret}=3.89 minutes (System1).

### Example 108

### 6-(3-Chloro-phenyl)-3-[3-(4-dimethylamino-piperidin-1-yl)-phenyl]-5-methyl-pyrazolo[1,5-a]pyrimidin-7-ylamine

The title compound is prepared as described in example 72; using {1-[3-(5-Amino-1H-pyrazol-4-yl)-phenyl]-piperidin-4-yl}-dimethyl-amine instead.

### Stage 108.1 {1-[3-(5-Amino-1H-pyrazol-4-yl)-phenyl]-piperidin-4-yl}-dimethyl-amine

The title compound is prepared as described in example 1, (Stage 1.2 and 1.4 and 1.5); using (3-bromo-phenyl)-acetonitrile and dimethyl-piperidin-4-yl-amine instead.

### Example 109

### 6-(3,4-Difluoro-phenyl)-3-[3-(4-dimethylamino-piperidin-1-yl)-phenyl]-5-methyl-pyrazolo[1,5-a]pyrimidin-7-ylamine

The title compound is prepared as described in example 72; using (1-[3-(5-Amino-1H-pyrazol-4-yl)-phenyl]-piperidin-4-yl}-dimethyl-amine (Example 108; Stage 108.1) and 2-(3,4-difluoro-phenyl)-3-oxo-butyronitrile (Example 98; Stage 98.1) instead.

### Example 110

### 6-(3-Chloro-phenyl)-5-methyl-3-(3,4,5-trimethoxy-phenyl)-pyrazolo[1,5-a]pyrimidin-7-ylamine

The title compound is prepared as described in example 72; using 4-(3,4,5-trimethoxy-phenyl)-2H-pyrazol-3-ylamine instead.

### Stage 110.1: 4-(3,4,5-Trimethoxy-phenyl)-2H-pyrazol-3-ylamine

The title compound is prepared as described in example 1, (Stage 1.4 and 1.2); using (3,4,5-trimethoxy-phenyl)-acetonitrile instead.

### Example 111

### 6-(3,4-Difluoro-phenyl)-5-methyl-3-(3,4,5-trimethoxy-phenyl)-pyrazolo[1,5-a]pyrimidin-7-ylamine

The title compound is prepared as described in example 72; using 4-(3,4,5-trimethoxy-phenyl)-2H-pyrazol-3-ylamine (Example 110; Stage 110.1) and 2-(3,4-difluoro-phenyl)-3-oxo-butyronitrile (Example 98; Stage 98.1) instead.

### Example 112

### 6-(3-Chloro-phenyl)-3-(3-methoxy-phenyl)-5-methyl-pyrazolo[1,5-a]pyrimidin-7-ylamine

The title compound is prepared as described in example 72; using 4-(3-Methoxy-phenyl)-2H-pyrazol-3-ylamine instead.

### Stage 112.1: 4-(3-Methoxy-phenyl)-2H-pyrazol-3-ylamine

The title compound is prepared as described in example 1, (Stage 1.4 and 1.2); using (3-methoxy-phenyl)-acetonitrile instead.

### Example 113

### 6-[7-Amino-3-(3,4-dimethoxy-phenyl)-pyrazolo[1,5-a]pyrimidin-6-yl]-pyridin-2-ol

The title compound is prepared as described in example 1; using 2-(6-Hydroxy-pyridin-2-yl)-3-oxo-propionitrile and 4-(3,4-Dimethoxy-phenyl)-2H-pyrazol-3-ylamine (Example 93; Stage 96.1) instead.

### Example 114

### 6-Benzyl-3-(3,4-dimethoxy-phenyl)-pyrazolo[1,5-a]pyrimidin-7-ylamine

The title compound is prepared as described in example 93; using 4-(3,4-Dimethoxy-phenyl)-2H-pyrazol-3-ylamine (Example 93; Stage 93.1) instead.

### Example 115

### 3-(3,4-Dimethoxy-phenyl)-6-(3-fluoro-benzyl)-pyrazolo[1,5-a]pyrimidin-7-ylamine

The title compound is prepared as described in example 114; using 2-(3-Fluoro-benzyl)-3-oxo-propionitrile instead.

### Example 116

### Tablets 1 Comprising Compounds of the Formula (I)

Tablets, comprising, as active ingredient, 50 mg of any one of the compounds of formula (I) mentioned in the preceding Examples 1-115 of the following composition are prepared using routine methods:

| Composition: | | |
|---|---|---|
| Active Ingredient | 50 | mg |
| Wheat starch | 60 | mg |
| Lactose | 50 | mg |
| Colloidal silica | 5 | mg |
| Talcum | 9 | mg |
| Magnesium stearate | 1 | mg |
| | 175 | mg |

Manufacture: The active ingredient is combined with part of the wheat starch, the lactose and the colloidal silica and the mixture pressed through a sieve. A further part of the wheat starch is mixed with the 5-fold amount of water on a water bath to form a paste and the mixture made first is kneaded with this paste until a weakly plastic mass is formed.

The dry granules are pressed through a sieve having a mesh size of 3 mm, mixed with a pre-sieved mixture (1 mm sieve) of the remaining corn starch, magnesium stearate and talcum and compressed to form slightly biconvex tablets.

### Example 117

### Tablets 2 Comprising Compounds of the Formula (I)

Tablets, comprising, as active ingredient, 100 mg of any one of the compounds of formula (I) of Examples 1-115 are prepared with the following composition, following standard procedures:

| Composition: | | |
|---|---|---|
| Active Ingredient | 100 | mg |
| Crystalline lactose | 240 | mg |
| Avicel | 80 | mg |
| PVPPXL | 20 | mg |
| Aerosil | 2 | mg |
| Magnesium stearate | 5 | mg |
| | 447 | mg |

Manufacture: The active ingredient is mixed with the carrier materials and compressed by means of a tabletting machine (Korsch EKO, Stempeldurchmesser 10 mm).

### Example 118

### Capsules

Capsules, comprising, as active ingredient, 100 mg of any one of the compounds of formula (I) given in Examples 1-115, of the following composition are prepared according to standard procedures:

| Composition: | | |
|---|---|---|
| Active Ingredient | 100 | mg |
| Avicel | 200 | mg |
| PVPPXL | 15 | mg |
| Aerosil | 2 | mg |
| Magnesium stearate | 1.5 | mg |
| | 318.5 | mg |

Manufacturing is done by mixing the components and filling them into hard gelatine capsules, size 1.

### Example 119

### Kinase Inhibition by Compounds of the Present Invention

Activity determinations of compounds of the preceding examples, using the testing method described above, with the following test compounds of formula (I) exhibit activity for the following kinases shown in Table 4 (An “x” indicates activity for that kinase). “Activity” as used herein is defined as having IC₅₀ values for kinase inhibition of 10 μM or less than:

TABLE 4

| Ex. | c-abl | Flt-3 | KDR | s-Scr | RET | EphB4 | c-kit | Cdk1 | Ins-R |
|---|---|---|---|---|---|---|---|---|---|
| 1 | x | — | x | — | x | x | x | — | X |
| 2 | — | — | — | — | — | — | — | X | — |
| 7 | — | — | — | x | — | — | — | — | — |
| 16 | — | — | — | — | — | — | — | — | X |
| 18 | — | — | — | — | — | — | — | X | X |
| 19 | x | — | — | x | — | — | — | — | — |
| 21 | — | — | — | — | — | — | x | — | — |
| 24 | — | — | — | — | — | — | x | — | — |
| 27 | — | — | — | x | — | — | — | — | — |
| 29 | — | — | x | — | x | x | — | — | — |
| 35 | x | — | — | — | x | — | — | — | — |
| 37 | — | — | — | — | — | — | — | X | — |
| 39 | — | x | — | — | x | — | — | — | — |
| 45 | — | — | — | — | — | x | — | — | — |
| 46 | x | — | x | — | — | x | — | X | — |
| 48 | x | — | — | x | x | x | — | — | — |
| 49 | — | x | — | — | — | — | — | — | — |
| 60 | x | — | — | — | — | x | — | X | — |
| 61 | — | — | x | — | — | — | — | — | — |
| 61a | — | — | — | — | x | — | — | — | — |

### Example 120

### Kinase Inhibition by Compounds of the Present Invention

Activity determinations of compounds of the preceding examples, using the testing method described above, with the following test compounds of formula (I) exhibit activity for the following kinases shown in Table 5. “Activity” as used herein is defined as having IC₅₀ values for kinase inhibition of 10 μM or less. Specifically, in the table:
“A” indicates an IC₅₀ value of below 1μM for that kinase.
“B” indicates an IC₅₀ value of in the range of 1μM-10 μM for that kinase.

“C” indicates an IC₅₀ value of at least 10μM:

TABLE 5

| Example | | | | | | | | | | |
|---|---|---|---|---|---|---|---|---|---|---|
| Nr. | EphB4 | Ins-R | IGF-1R | Flt-3 | HER-1 | CDK1/B | Tek | c-scr | C-abl | KDR |
| 72 | A | B | C | C | B | C | B | A | A | A |
| 73 | A | C | C | C | B | C | B | A | A | A |
| 74 | A | — | — | — | — | — | — | — | — | — |
| 75 | A | — | — | — | — | — | — | — | A | B |
| 76 | A | B | — | A | A | C | A | — | — | A |
| 77 | A | — | — | A | — | — | A | — | — | A |
| 78 | A | — | — | A | — | — | B | — | — | A |
| 79 | A | — | C | C | B | C | C | A | A | B |
| 80 | A | C | C | B | C | C | B | A | A | B |
| 81 | A | — | A | C | C | C | A | A | A | A |
| 82 | A | — | C | C | C | C | C | A | A | C |
| 83 | A | C | C | C | C | C | C | A | A | C |
| 84 | A | — | C | C | C | C | C | A | A | B |
| 85 | B | — | C | C | C | C | C | A | A | C |
| 86 | B | — | C | C | C | C | B | A | A | A |
| 87 | — | — | C | C | C | C | C | A | A | B |
| 88 | A | C | C | C | C | C | C | A | A | B |
| 89 | A | — | C | C | C | C | B | A | A | B |
| 90 | A | C | C | C | C | C | B | A | A | B |
| 91 | A | — | C | C | C | C | C | C | A | B |
| 92 | A | — | C | C | C | C | C | — | — | B |
| 93 | A | — | C | C | C | C | C | A | A | C |
| 94 | A | — | C | C | C | C | C | A | C | C |
| 95 | A | — | C | C | C | C | C | B | A | C |
| 96 | A | — | C | C | B | C | C | A | A | C |
| 97 | A | — | C | C | C | C | C | A | A | B |
| 98 | A | — | C | C | C | C | B | A | A | C |
| 99 | A | — | C | C | C | C | C | A | A | C |
| 100 | A | — | C | C | C | C | C | A | A | B |
| 101 | A | — | C | C | C | C | C | A | A | B |
| 102 | A | — | C | C | C | C | B | A | A | C |
| 103 | A | — | C | C | C | C | C | A | A | C |
| 104 | — | — | — | — | — | — | — | — | — | — |
| 105 | — | — | — | — | — | — | — | — | — | — |
| 106 | A | — | C | C | C | C | C | A | A | C |
| 107 | A | — | C | C | C | C | C | A | A | C |

### Example 121

### Inhibition of the Autophosphorylation of the EphB4 Kinase by Compounds of the Present Invention

Compounds of the preceding examples, using the testing method described above, with the following test compounds of formula (I) exhibit activity for the inhibition of the autophosphorylation of the EphB4 kinase (cellular assay). (An “x” indicates activity for that kinase). “Activity” as used herein is defined as having IC₅₀ values for kinase inhibition of 1 μM or less than:

TABLE 6

| | | EphB4 |
|---|---|---|
| Example | Compound | ELISA IC₅₀ |
| 80 | BAW209 | X |
| 83 | BBD149 | X |
| 90 | BCH454 | X |
| 93 | BCT421 | X |
| 100 | BDN800 | X |

1. A compound of formula (I): 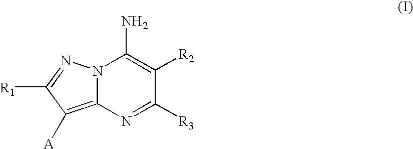
wherein:
R₂ is H; substituted or unsubstituted aryl; substituted or unsubstituted heteroaryl; an aliphatic residue; a functional group; or a substituted or unsubstituted aryl, substituted or unsubstituted heteroaryl or aliphatic residue which is connected by one connecting group or atom to the pyrazolo[1,5a]pyrimidinyl ring;
R₃ can be H, substituted or unsubstituted aryl, heteroaryl, an aliphatic residue, a functional group, or an aliphatic residue which may be connected by a connecting group or atom to the pyrazolo[1,5a]pyrimidinyl ring,
at least one of R₂ or R₃ is substituted or unsubstituted aryl; substituted or unsubstituted heteroaryl; or a substituted or unsubstituted heteroaryl or substituted or unsubstituted aryl residue which is connected by one connecting group or atom to the pyrazolo[1,5a]pyrimidinyl ring, and provided that both R₂ and A cannot both be unsubstituted phenyl;
A is H, halogen, an aliphatic moiety, a functional group, substituted or unsubstituted aryl or heteroaryl; and
R₁ is H, halogen or lower alkyl,
or a pharmaceutically acceptable salt thereof.

2. A compound according to claim 1, wherein:
R₂ is H; lower alkyl; cycloalkyl; benzyl; benzo thienyl, indyl substituted by lower alkyl, pyridyl or thiazolyl optionally substituted by lower alkyl; unsubstituted phenyl or phenyl substituted by one or two substituents chosen from the group consisting of; halo, hydroxy, alkoxy, benzyloxy, cycloalkyl, amino, acetyl amino, lower alkyl sulfonamide and benzene sulfonamide substituted by one or two halo;
R3 is H; lower alkyl optionally substituted by halo; phenyl, pyridyl, or oxazolyl;
A is
(a) H; halo; benzothienyl; pyridyl; methyl piperazinyl phenoxyl; indolyl substituted with lower alkyl;
(b) phenyl which is unsubstituted or substituted with one or more of the substituents chosen from the group consisting of; mono-, di- or tri-lower alkoxy, di-lower alkylaminyl, morpholinyl which is optionally di-substituted by alkyl,
piperazinyl which is substituted with one or more of the substituents chosen from the group consisting of lower alkyl, lower alkoxy, lower alkyl piperazinyl, pyrrolidinyl, dialkyl aminyl and lower alkanol; and
R₁ is H;
and provided that both R₂ and A cannot both be unsubstituted phenyl.

3. A compound of the formula (I), according to claim 1, wherein
A is H; a halo; or aryl or heterocyclyl,
wherein the aryl or heterocyclyl may be substituted or unsubstituted with up to 4, preferably up to 2 substituents, wherein the substituents are the same or different and are independently selected from halo; hydroxy; amino; amino lower alkyl; amino lower alkoxy; lower alkyl; lower alkoxy; substituted or unsubstituted sulfonamide; carbamates; R₄R₅, wherein R₄ and R₅ can be the same or different and are independently H; lower alkyl; or R₄ and R₅ together with the N atom form a 3- to 8-membered heterocyclic ring containing 1-4 nitrogen, oxygen or sulfur atoms where when R₄ and R₅ together with the N form an heterocyclic ring, said ring may be substituted with 1, 2 or more of any of the substituents described herein, preferably piperazinyl, pyrrolidinyl, alkyl such as methyl, or hydroxy alkyl;
R₂ is H; C₁-C₃ lower alkyl; aryl; heterocyclyl, wherein the aryl or heterocyclyl may be substituted or unsubstituted with up to 4, preferably up to 2 substituents, wherein the substituents are the same or different and are independently selected from halo; hydroxy; amino; amino lower alkyl; C₁-C₃ lower alkyl; alkoxy; sulfoamino; substituted or unsubstituted benzosulfonamide; substituted or unsubstituted sulfonate; substituted or unsubstituted ureas or carbamates;
R₃ is H; C₁-C₃ alkyl, methyl; phenyl; pyridinyl or oxaz-5-yl;
or a pharmaceutically acceptable salt thereof.

4. A compound of the formula (I), according to claim 3, wherein
A is H; Br; phenyl, benzyl; pyridinyl; indolyl; benzothiophenyl.

5. A compound of the formula (I), according to claim 3, wherein
the heteroring formed by R₄ and R₅ together with the N is selected from morpholinyl, which can be unsubstituted or substituted with methyl or dimethyl; piperazinyl which can be unsubstituted or substituted with 1, 2 or 3 substituents prefereably methyl, oxy or ethanol;
or piperadinyl which can be unsubstituted or substituted with 1, 2 or 3 substituents prefereably pyrrolidinyl, amine, alkyl amine, methyl amine, dialkyl amine, dimethylamine or diethylamine.

6. A method for treating a protein kinase dependent disease comprising administering to a mammal in need thereof a compound of the formula (I): 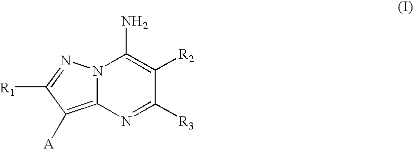
wherein:
R₂ is H; substituted or unsubstituted aryl; substituted or unsubstituted heteroaryl; substituted or unsubstituted aliphatic residue; a functional group; or a substituted or unsubstituted aryl, substituted or unsubstituted heteroaryl or substituted or unsubstituted aliphatic residue which is connected by one connecting group or atom to the pyrazolo[1,5a]pyrimidinyl ring;
R₃ can be H, substituted or unsubstituted aryl, substituted or unsubstituted heteroaryl, substituted or unsubstituted aliphatic residue, a functional group, or a substituted or unsubstituted aliphatic residue which may be connected by a connecting group or atom to the pyrazolo[1,5a]pyrimidinyl ring,
at least one of R₂ or R₃ is substituted or unsubstituted aryl; substituted or unsubstituted heteroaryl; or a substituted or unsubstituted heteroaryl or substituted or unsubstituted aryl residue which is connected by one connecting group or atom to the pyrazolo[1,5a]pyrimidinyl ring;
A is H, halogen, an aliphatic moiety, a functional group, substituted or unsubstituted aryl or substituted or unsubstituted heteroaryl; and
R₁ is H, halogen or lower alkyl,
or pharmaceutically acceptable salts thereof.

7. A method according to claim 6, comprising a compound of formula (I) wherein:
R₂ is H; lower alkyl; cycloalkyl; benzyl; benzo thienyl, indyl substituted by lower alkyl, pyridyl or thiazolyl optionally substituted by lower alkyl; unsubstituted phenyl or phenyl substituted by one or two substituents chosen from the group consisting of; halo, hydroxy, alkoxy, benzyloxy, cycloalkyl, amino, acetyl amino, lower alkyl sulfonamide and benzene sulfonamide substituted by one or two halo;
R3 is H; lower alkyl optionally substituted by halo; phenyl, pyridyl, or oxazolyl;
A is
(a) H; halo; benzothienyl; pyridyl; methyl piperazinyl phenoxyl; indolyl substituted with lower alkyl;
(b) phenyl which is unsubstituted or substituted with one or more of the substituents chosen from the group consisting of; mono-, di- or tri-lower alkoxy, di-lower alkylaminyl, morpholinyl which is optionally di-substituted by alkyl,
piperazinyl which is substituted with one or more of the substituents chosen from the group consisting of lower alkyl, lower alkoxy, lower alkyl piperazinyl, pyrrolidinyl, dialkyl aminyl and lower alkanol; and
R₁ is H,
or pharmaceutically acceptable salts thereof for treating a protein kinase dependent disease.

8. A method according to claim 6, comprising a compound of formula (I) wherein:
A is H; a halo; aryl or heterocyclyl, wherein the aryl or heterocyclyl may be substituted or unsubstituted with up to 4, preferably up to 2 substituents, wherein the substituents are the same or different and are independently selected from halo; hydroxy; amino; amino lower alkyl; amino lower alkoxy; lower alkyl; lower alkoxy; substituted or unsubstituted sulfonamide; carbamates; R₄R₅, wherein R₄ and R₅ can be the same or different and are independently H; lower alkyl; or R₄ and R₅ together with the N atom form a 3- to 8-membered heterocyclic ring containing 1-4 nitrogen, oxygen or sulfur atoms where when R₄ and R₅ together with the N form an heterocyclic ring, said ring may be substituted with 1, 2 or more of any of the substituents described herein, preferably piperazinyl, pyrrolidinyl, alkyl such as methyl, or hydroxy alkyl such as ethanyl;
R₂ is H, C₁-C₃ lower alkyl; aryl or heterocyclyl, wherein the aryl or heterocyclyl may be substituted or unsubstituted with up to 4, preferably up to 2 substituents, wherein the substituents are the same or different and are independently selected from halo; hydroxy; amino; amino lower alkyl; C₁-C₃ lower alkyl; alkoxy; sulfoamino; substituted or unsubstituted benzosulfonamide; substituted or unsubstituted sulfonate; substituted or unsubstituted ureas or carbamates;
R₃ is H; C₁-C₃ alkyl, methyl; phenyl; pyridinyl or oxaz-5-yl;
or a pharmaceutically acceptable salt thereof.

9. A method according to claim 8 comprising a compound of the formula (I), wherein
A is H; Br; phenyl, benzyl; pyridinyl; indolyl; benzothiophenyl.

10. A method according to claim 8 comprising a compound of the formula (I), wherein the heteroring formed by R₄ and R₅ together with the N is selected from morpholinyl, which can be unsubstituted or substituted with methyl or dimethyl; piperazinyl which can be unsubstituted or substituted with 1, 2 or 3 substituents prefereably methyl, oxy or ethanol;
or piperadinyl which can be unsubstituted or substituted with 1, 2 or 3 substituents prefereably pyrrolidinyl, amine, alkyl amine, methyl amine, dialkyl amine, dimethylamine or diethylamine.

11. A method according to claim 6, wherein the kinase dependent disease is one depending on c-Abl, Bcr-Abl, c-Kit, c-Raf, Fit-1, Flt-3, Her-1, KDR, PDGFR-kinase, c-Src, RET-receptor kinase, FGF-R1, FGF-R2, FGF-R3, FGF-R4, Ephrin receptor kinases (e.g., EphB2 kinase, EphB4 kinase and related Eph kinases), casein kinases (CK-1, CK-2, G-CK), Pak, ALK, ZAP70, Jak1, Jak2, Axl, Cdk1, cdk4, cdk5, Met, FAK, Pyk2, Syk, Insulin receptor kinase, Tie-2 or costitutively activating mutations of kinases (activating kinases) such as of Bcr-Abl, c-Kit, c-Raf, Flt-3, FGF-R3, PDGF-receptors, RET, and Met and (especially aberrantly highly expressed or activated) kinase-dependent disease or disease dependent on the activation of the kinase pathways, or a disease dependent on any two or more of the kinases just mentioned.

12. A method according to claim 6 wherein the kinase dependent disease is one depending on c-abl, Flt-3, KDR, c-Src, RET, EphB4, c-kit, cdk1, FGFR-1, c-raf, Her-1, Ins-R or Tek.

13. A method according to claim 6, wherein the disease to be treated is a proliferative disease, preferably a benign or especially malignant tumor, more preferably carcinoma of the brain, kidney, liver, adrenal gland, bladder, breast, stomach (especially gastric tumors), ovaries, colon, rectum, prostate, pancreas, lung, vagina, thyroid, sarcoma, glioblastomas, multiple myeloma or gastrointestinal cancer, especially colon carcinoma or colorectal adenoma, or a tumor of the neck and head, an epidermal hyperproliferation, especially psoriasis, prostate hyperplasia, a neoplasia, especially of epithelial character, preferably mammary carcinoma, or a leukemia.

14. A method according to claim 6, wherein the disease to be treated is triggered by persistent angiogenesis, such as psoriasis; Kaposi's sarcoma; restenosis, e.g., stent-induced restenosis; endometriosis; Crohn's disease; Hodgkin's disease; leukemia; arthritis, such as rheumatoid arthritis; hemangioma; angiofibroma; eye diseases, such as diabetic retinopathy and neovascular glaucoma; renal diseases, such as glomerulonephritis; diabetic nephropathy; malignant nephrosclerosis; thrombotic microangiopathic syndromes; transplant rejections and glomerulopathy; fibrotic diseases, such as cirrhosis of the liver; mesangial cell-proliferative diseases; arteriosclerosis; injuries of the nerve tissue; and for inhibiting the re-occlusion of vessels after balloon catheter treatment, for use in vascular prosthetics or after inserting mechanical devices for holding vessels open, such as, e.g., stents, as immunosuppressants, as an aid in scar-free wound healing, and for treating age spots and contact dermatitis.

15. A compound selected from the group consisting of:
3-{7-Amino-3-[4-(4-methyl-piperazin-1-yl)-phenyl]-pyrazolo[1,5-a]pyrimidin-6-yl}-phenol;
6-(3-benzyloxy-phenyl)-3-[4-(4-methyl-piperazin-1-yl)-phenyl]-pyrazolo[1,5-a]pyrimidin-7-yl}-phenol;
6-(3-Methoxy-phenyl)-3-[4-(4-methyl-piperazin-1-yl)-phenyl]-pyrazolo[1,5-a]pyrimidin-7-ylamine;
6-(3,5-Dimethoxy-phenyl)-3-[4-(4-methyl-piperazin-1-yl)-phenyl]-pyrazolo[1,5-a]pyrimidin-7-ylamine;
6-(3-Benzyloxy-phenyl)-3-[4-(4-methyl-piperazin-1-yl)-phenyl]-pyrazolo[1,5-a]pyrimidin-7-ylamine;
6-(4-Chloro-phenyl)-3-[4-(4-methyl-piperazin-1-yl)-phenyl]-pyrazolo[1,5-a]pyrimidin-7-ylamine;
6-(3-Chloro-phenyl)-3-[4-(4-methyl-piperazin-1-yl)-phenyl]-pyrazolo[1,5-a]pyrimidin-7-ylamine;
3-[4-(4-Methyl-piperazin-1-yl)-phenyl]-6-phenyl-pyrazolo[1,5-a]pyrimidin-7-ylamine;
5-Methyl-3-[4-(4-methyl-piperazin-1-yl)-phenyl]-6-phenyl-pyrazolo[1,5-a]pyrimidin-7-ylamine;
6-Methyl-3-[4-(4-methyl-piperazin-1-yl)-phenyl]-5-phenyl-pyrazolo[1,5-a]pyrimidin-7-ylamine;
N-{4-[7-Amino-3-(4-dimethylamino-phenyl)-pyrazolo[1,5-a]pyrimidin-6-yl]-phenyl}-2,3-dichloro-benzenesulfonamide;
4-Chloro-benzenesulfonic acid 4-[7-amino-3-(4-dimethylamino-phenyl)-pyrazolo[1,5-a]pyrimidin-6-yl]-phenyl ester;
6-(4-Methoxy-phenyl)-5-methyl-3-phenyl-pyrazolo[1,5-a]pyrimidin-7-ylamine;
3-(4- Methoxy-phenyl)-5-methyl-6-phenyl-pyrazolo[1,5-a]pyrimidin-7-ylamine;
6-(4-Bromo-phenyl)-3-(4-methoxy-phenyl)-5-methyl-pyrazolo[1,5-a]pyrimidin-7-ylamine;
6-(4-Bromo-phenyl)-5-methyl-3-phenyl-pyrazolo[1,5-a]pyrimidin-7-ylamine;
6-(2,6-Dichloro-phenyl)-3-phenyl-pyrazolo[1,5-a]pyrimidin-7-ylamine;
3-(3-Methoxy-phenyl)-6-phenyl-pyrazolo[1,5-a]pyrimidin-7-ylamine;
3-Bromo-5-phenyl-pyrazolo[1,5-a]pyrimidin-7-ylamine;
6-Benzo[b]thiophen-3-yl-3-[4-(4-methyl-piperazin-1-yl)-phenyl]-pyrazolo[1,5-a]pyrimidin-ylamine;
3-(4-Bromo-phenyl)-5-phenyl-pyrazolo[1,5-a]pyrimidin-7-ylamine;
3-[4-(4-Methyl-piperazin-1-yl)-phenyl]-6-thiophen-3-yl-pyrazolo[1,5-a]pyrimidin-7-ylamine;
3-Benzo[b]thiophen-3-yl-6-(3-methoxy-phenyl)-pyrazolo[1,5-a]pyrimidin-7-ylamine;
6-Benzo-3-[4-(4-methyl-piperazin-1-yl)-phenyl]-pyrazolo[1,5-a]pyrimidin-7-ylamine;
6-(3-Methoxy-phenyl)-3-[3-(4-methyl-piperazin-1-yl)-phenyl]-pyrazolo[1,5-a]pyrimidin-7-ylamine;
6-(1-Methyl-1H-indol-3-yl)-3-[4-(4-methyl-piperazin-1-yl)-phenyl]-pyrazolo[1,5-a]pyrimidin-7-ylamine;
6-(4-Methoxy-phenyl)-3-[4-(4-methyl-piperazin-1-yl)-phenyl]-pyrazolo[1,5-a]pyrimidin-7-ylamine;
6-(2-Methoxy-phenyl)-3-[4-(4-methyl-piperazin-1-yl)-phenyl]-pyrazolo[1,5-a]pyrimidin-7-ylamine;
6-(3-Methoxy-phenyl)-3-pyridin-3-yl-pyrazolo[1,5-a]pyrimidin-7-ylamine;
3-{7-Amino-3-[3-(4-methyl-piperazin-1-yl)-phenyl]-pyrazolo[1,5-a]pyrimidin-6-yl}-phenol;
6-(3-Benzyloxy-phenyl)-3-[2-methoxy-5-(4-methyl-piperazin-1-yl)-phenyl]-pyrazolo[1,5-a]pyrimidin-7-ylamine;
3-{7-Amino-3-[2-methoxy-5-(4-methyl-piperazin-1-yl)-phenyl]-pyrazolo[1,5-a]pyrimidin-6-yl}-phenol;
6-(2-Benzyloxy-phenyl)-3-[4-(4-methyl-piperazin-1-yl)-phenyl]-pyrazolo[1,5-a]pyrimidin-7-ylamine;
2-{7-Amino-3-[4-(4-methyl-piperazin-1-yl)-phenyl]-pyrazolo[1,5-a]pyrimidin-6-yl}-phenol;
6-(4-Benzyloxy-phenyl)-3-[4-(4-methyl-piperazin-1-yl)-phenyl]-pyrazolo[1,5-a]pyrimidin-7-ylamine;
4-{7-Amino-3-[4-(4-methyl-piperazin-1-yl)-phenyl]-pyrazolo[1,5-a]pyrimidin-6-yl}-phenol;
6-(2-Benzyloxy-phenyl)-3-[3-(4-methyl-piperazin-1-yl)-phenyl]-pyrazolo[1,5-a]pyrimidin-7-ylamine;
2-{7-Amino-3-[3-(4-methyl-piperazin-1-yl)-phenyl]-pyrazolo[1,5-a]pyrimidin-6-yl)-phenol;
6-(4-Benzyloxy-phenyl)-3-[3-(4-methyl-piperazin-1-yl)-phenyl]-pyrazolo[1,5-a]pyrimidin-7-ylamine;
4-{7-Amino-3-[3-(4-methyl-piperazin-1-yl)-phenyl]-pyrazolo[1,5-a]pyrimidin-6-yl}-phenol;
6-(2-Benzyloxy-phenyl)-3-[2-methoxy-5-(4-methyl-piperazin-1-yl)-phenyl]-pyrazolo[1,5-a]pyrimidin-7-ylamine;
2-{7-Amino-3-[2-methoxy-5-(4-methyl-piperazin-1-yl)-phenyl]-pyrazolo[1,5-a]pyrimidin-6-yl}-phenol;
6-(4-Benzyloxy-phenyl)-3-[2-methoxy-5-(4-methyl-piperazin-1-yl)-phenyl]-pyrazolo[1,5-a]pyrimidin-7-ylamine;
4-{7-Amino-3-[2-methoxy-5-(4-methyl-piperazin-1-yl)-phenyl]-pyrazolo[1,5-a]pyrimidin-6-yl}-phenol;
6-(3-Benzyloxy-phenyl)-3-[1-methyl-1H-indol-3-yl)-pyrazolo[1,5-a]pyrimidin-7-ylamine;
3-[7-Amino-3-(1-methyl-1H-indol-3-yl)-pyrazolo[1,5-a]pyrimidin-6-yl]-phenol;
3-[7-Amino-3-pyridin-3-yl-pyrazolo[1,5-a]pyrimidin-6-yl]-phenol;
6-(3-Benzyloxy-phenyl)-3-(2-methoxy-phenyl)-pyrazolo[1,5-a]pyrimidin-7-ylamine;
3-[7-Amino-3-(2-methoxy-phenyl)-pyrazolo[1,5-a]pyrimidin-6-yl]-phenol;
3-[3-(4-Methyl-piperazin-1-yl)-phenyl]-6-thiophen-3-yl-pyrazolo[1,5-a]pyrimidin-7-ylamine;
3-(2-Methoxy-5-(4-methyl-piperazin-1-yl)-phenyl]-6-thiophen-3-yl-pyrazolo[1,5-a]pyrimidin-7-ylamine;
3-[4-(4-Methyl-piperazin-1-yl)-phenyl]-6-pyridin-4-yl-pyrazolo[1,5-a]pyrimidin-7-ylamine;
6-(3-Amino-phenyl)-3-[3-(4-methyl-piperazin-1-yl)-phenyl]-pyrazolo[1,5-a]pyrimidin-7-ylamine;
6-(3-Amino-phenyl)-3-[4-(4-methyl-piperazin-1-yl)-phenyl]-pyrazolo[1,5-a]pyrimidin-7-ylamine;
6-(2-Amino-phenyl)-3-[4-(4-methyl-piperazin-1-yl)-phenyl]-pyrazolo[1,5-a]pyrimidin-7-ylamine;
3-[4-(4-Methyl-piperazin-1-yl)-phenyl]-6-(4-methyl-thiazol-2-yl)-pyrazolo[1,5-a]pyrimidin-7-ylamine;
6-Benzo[b]thiophen-3-yl-3-[2-methoxy-5-(4-methyl-piperazin-1-yl)-phenyl]-pyrazolo[1,5-a]pyrimidin-7-ylamine;
6-Benzo[b]thiophen-3-yl-3-[4-methoxy-phenyl)-pyrazolo[1,5-a]pyrimidin-7-ylamine;
3-(3-Methoxy-phenyl)-6-thiophen-3-yl-pyrazolo[1,5-a]pyrimidin-7-ylamine;
6-(3-Benzyloxy-phenyl)-3-(3-methoxy-phenyl)-pyrazolo[1,5-a]pyrimidin-7-ylamine;
3-[7-Amino-3-(3-methoxy-phenyl)-pyrazolo[1,5-a]pyrimidin-6-yl]-phenol;
(4-{7-Amino-3-[4-(4-methyl-piperazin-1-yl)-phenyl]-pyrazolo[1,5-a]pyrimidin-6-yl}-phenyl)-carbamic acid ethyl ester
6-(3-Chloro-phenyl)-5-methyl-3-[3-(4-methyl-piperazin-1-yl)-phenyl]-pyrazolo[1,5-a]pyrimidin-7-ylamine;
6-(3-Chloro-phenyl)-5-methyl-3-[4-(4-methyl-piperazin-1-yl)-phenyl]-pyrazolo[1,5-a]pyrimidin-7-ylamine;
6-(3-Chloro-phenyl)-3-[2-methoxy-5-(4-methyl-piperazin-1-yl)-phenyl]-5-methyl-pyrazolo[1,5-a]pyrimidin-7-ylamine;
6-(3-Chloro-phenyl)-3-[2-methoxy-4-(4-methyl-piperazin-1-yl)-phenyl]-5-methyl-pyrazolo[1,5-a]pyrimidin-7-ylamine;
3-(7-Amino-3-[2-methoxy-4-(4-methyl-piperazin-1-yl)-phenyl]-pyrazolo[1,5-a]pyrimidin-6-yl}-phenol; 6-(2-Chloro-phenyl)-3-[4-(4-methyl-piperazin-1-yl)-phenyl]-pyrazolo[1,5-a]pyrimidin-7-ylamine;
6-(2-Chloro-phenyl)-3-[3-(4-methyl-piperazin-1-yl)-phenyl]-pyrazolo[1,5-a]pyrimidin-7-ylamine;
6-(4-Fluoro-phenyl)-5-methyl-3-[4-(4-methyl-piperazin-1-yl)-phenyl]-pyrazolo[1,5-a]pyrimidin-7-ylamine;
6-(4-Fluoro-phenyl)-5-methyl-3-[3-(4-methyl-piperazin-1-yl)-phenyl]-pyrazolo[1,5-a]pyrimidin-7-ylamine;
6-(3-Chloro-phenyl)-5-methyl-3-{3-[4-(1-methyl-piperidin-4-yl)-piperazin-1-yl]-phenyl}-pyrazolo[1,5-a]pyrimidin-7-ylamine;
6-(3-Chloro-4-fluoro-phenyl)-5-methyl-3-[3-(4-methyl-piperazin-1-yl)-phenyl]pyrazolo[1,5-a]pyrimidin-7-amine;
6-(3-Chloro-4-fluoro-phenyl)-5-methyl-3-[4-(4-methyl-piperazin-1-yl)-phenyl]-pyrazolo[1,5-a]pyrimidin-7-ylamine;
6-(3-Bromo-phenyl)-5-methyl-3-[3-(4-methyl-piperazin-1-yl)-phenyl]-pyrazolo[1,5-a]pyrimidin-7-ylamine;
6-(3-Bromo-benzyl)-3-[3-(4-methyl-piperazin-1-yl)-phenyl]-pyrazolo[1,5-a]pyrimidin-7-ylamine;
6-(3-Bromo-phenyl)-3-[3-(4-methyl-piperazin-1-yl)-phenyl]-pyrazolo[1,5-a]pyrimidin-7-ylamine;
6-(3-Chloro-phenyl)-5-methyl-3-(3-morpholin-4-yl-phenyl)-pyrazolo[1,5-a]pyrimidin-7-ylamine;
6-(3-Chloro-phenyl)-3-(4-methoxy-phenyl)-5-methyl-pyrazolo[1,5-a]pyrimidin-7-ylamine;
6-(3-Chloro-phenyl)-3-[3-((2R,6S)-2,6-dimethyl-morpholin-4-yl)-phenyl]-5-methyl-pyrazolo[1,5-a]pyrimidin-7-ylamine;
2-(4-{3-[7-Amino-6-(3-chloro-phenyl)-5-methyl-pyrazolo[1,5-a]pyrimidin-3-yl]-phenyl}-piperazin-1-yl)-ethanol;
6-Benzyl-3-[3-(4-methyl-piperazin-1-yl)-phenyl]-pyrazolo[1,5-a]pyrimidin-7-ylamine;
6-(3-Chloro-phenyl)-3-(3,4-dimethoxy-phenyl)-5-fluoromethyl-pyrazolo[1,5-a]pyrimidin-7-ylamine;
6-(3-Chloro-phenyl)-3-(3,4-dimethoxy-phenyl)-5-methyl-pyrazolo[1,5-a]pyrimidin-7-ylamine;
6-(3-Chloro-4-fluoro-phenyl)-3-(3,4-dimethoxy-phenyl)-5-methyl-pyrazolo[1,5-a]pyrimidin-7-ylamine;
6-(3-Chloro-4-fluoro-phenyl)-3-(4-methoxy-phenyl)-5-methyl-pyrazolo[1,5-a]pyrimidin-7-ylamine;
6-(4-Fluoro-phenyl)-3-(4-methoxy-phenyl)-5-methyl-pyrazolo[1,5-a]pyrimidin-7-ylamine;
2-(4-{3-[7-Amino-6-(4-fluoro-phenyl)-5-methyl-pyrazolo[1,5-a]pyrimidin-3-yl]-phenyl}-piperazin-1-yl)-ethanol;
6-(3,4-Difluoro-phenyl)-5-methyl-3-[3-(4-methyl-piperazin-1-yl)-phenyl]-pyrazolo[1,5-a]pyrimidin-7-ylamine;
6-(3,4-Difluoro-phenyl)-3-(3,4-dimethoxy-phenyl)-5-methyl-pyrazolo[1,5-a]pyrimidin-7-ylamine;
2-(4-{3-[7-Amino-6-(3-chloro-4-fluoro-phenyl)-5-methyl-pyrazolo[1,5-a]pyrimidin-3-yl}-phenyl}-piperazin-1-yl)-ethanol;
2-(4-{3-[7-Amino-6-(3,4-difluoro-phenyl)-5-methyl-pyrazolo[1,5-a]pyrimidin-3-yl]-phenyl}-piperazin-1-yl)-ethanol;
6-(3-Chloro-phenyl)-5-methyl-3-[3-(4-pyrrolidin-1-yl-piperidin-1-yl)-phenyl]-pyrazolo[1,5-a]pyrimidin-7-ylamine;
6-(4-Fluoro-phenyl)-5-methyl-3-[3-(4-pyrrolidin-1-yl-piperidin-1-yl)-phenyl]-pyrazolo[1,5-a]pyrimidin-7-ylamine;
6-(3-Chloro-phenyl)-3-[3-(4-diethylamino-piperidin-1-yl)-phenyl]-5-methyl-pyrazolo[1,5-a]pyrimidin-7-ylamine;
3-[3-(4-Diethylamino-piperidin-1-yl)-phenyl]-6-(4-fluoro-phenyl)-5-methyl-pyrazolo[1,5-a]pyrimidin-7-ylamine;
6-(4-Fluoro-phenyl)-5-methyl-3-[3-(4-methyl-4-oxy-piperazin-1-yl)-phenyl]-pyrazolo[1,5-a]pyrimidin-7-ylamine;
6-(4-Fluoro-phenyl)-5-methyl-3-[3-(4-methyl-1,4-dioxy-piperazin-1-yl)-phenyl]-pyrazolo[1,5-a]pyrimidin-7-ylamine;
6-(3-Chloro-phenyl)-3-[3-(4-dimethylamino-piperidin-1-yl)-phenyl]-5-methyl-pyrazolo[1,5-a]pyrimidin-7-ylamine;
6-(3,4-Difluoro-phenyl)-3-[3-(4-dimethylamino-piperidin-1-yl)-phenyl]-5-methyl-pyrazolo[1,5-a]pyrimidin-7-ylamine;
6-(3-Chloro-phenyl)-5-methyl-3-(3,4,5-trimethoxy-phenyl)-pyrazolo[1,5-a]pyrimidin-7-ylamine; 6-(3,4-Difluoro-phenyl)-5-methyl-3-(3,4,5-trimethoxy-phenyl)-pyrazolo[1,5-a]pyrimidin-7-ylamine;
6-(3-Chloro-phenyl)-3-(3-methoxy-phenyl)-5-methyl-pyrazolo[1,5-a]pyrimidin-7-ylamine;
6-[7-Amino-3-(3,4-dimethoxy-phenyl)-pyrazolo[1,5-a]pyrimidin-6-yl]-pyridin-2-ol;
6-Benzyl-3-(3,4-dimethoxy-phenyl)-pyrazolo[1,5-a]pyrimidin-7-ylamine;
3-(3,4-Dimethoxy-phenyl)-6-(3-fluoro-benzyl)-pyrazolo[1,5-a]pyrimidin-7-ylamine; and pharmaceutically acceptable salts thereo.

16. A pharmaceutical composition comprising a compound according to claim 1.

17. A pharmaceutical composition comprising a compound according to claim 1 and an acceptable pharmaceutical carrier.

18. A process to prepare a compound according to claim 1 comprising:
(a) reacting a nitrile, A-CH₂—C≡N, with ethyl formate in the presence of an organic solvent to form a substituted 3-oxo-propionitrile,
(b) condensing the substituted 3-oxo-propionitriles of step (a) with hydrazine monohydrate in an organic solvent to form a 2H-pyrazol-3-ylamine of formula (III): 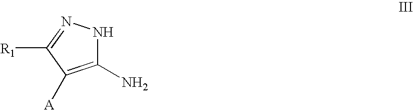
(d) formylating a substituted nitrile in the presence of ethanolate and formic acid ethyl ester to prepare a 3-oxo-propionitrile of formula (II): 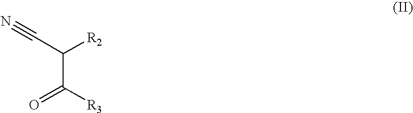
(c) condensing the 3-oxo-propionitrile of formula (II) and the 2H-pyrazol-3-ylamines of formula (III) in the presence of an organic solvent to form a compound of formula (I).
